(19) United States
(12) Patent Application Publication
(10) Pub. No.: US 2026/0120824 A1
(43) Pub. Date: Apr. 30, 2026

(54) HEALTH INFORMATION USER INTERFACES

(71) Applicant: Apple Inc., Cupertino, CA (US)

(72) Inventor: Matthew W. CROWLEY, Sunnyvale, CA (US)

(21) Appl. No.: 19/340,642

(22) Filed: Sep. 25, 2025

Related U.S. Application Data
(60) Provisional application No. 63/700,493, filed on Sep. 27, 2024.
(60) Provisional application No. 63/737,509, filed on Dec. 20, 2024.

(51) Int. Cl. G16H 10/20; G16H 20/30; G16H 40/67; G16H 50/30

(52) U.S. Cl. CPC G16H 10/20; G16H 20/30; G16H 40/67; G16H 50/30

(57) ABSTRACT

The present disclosure generally relates to tracking and providing user health information.

### CROSS-REFERENCE TO RELATED APPLICATIONS

This application claims priority to U.S. Provisional Patent Application No. 63/737, 509, entitled “HEALTH INFORMATION USER INTERFACES,” filed on Dec. 20, 2024, and U.S. Provisional Patent Application No. 63/700,493, entitled “HEALTH INFORMATION USER INTERFACES,” filed on Sep. 27, 2024, the contents of which are hereby incorporated by reference in their entirety.

### FIELD

The present disclosure relates generally to computer user interfaces, and more specifically to techniques for tracking and presenting user health information.

### BACKGROUND

Electronic devices can be used to track health metrics and display information pertaining to health metrics of a user.

### BRIEF SUMMARY

Some techniques for tracking and presenting user health information using electronic devices, however, are generally cumbersome and inefficient. For example, some existing techniques use a complex and time-consuming user interface, which may include multiple key presses or keystrokes. Existing techniques require more time than necessary, wasting user time and device energy. This latter consideration is particularly important in battery-operated devices.

Accordingly, the present technique provides electronic devices with faster, more efficient methods and interfaces for tracking and presenting user health information. Such methods and interfaces optionally complement or replace other methods for tracking and presenting user health information. Such methods and interfaces reduce the cognitive burden on a user and produce a more efficient human-machine interface. For battery-operated computing devices, such methods and interfaces conserve power and increase the time between battery charges.

In some embodiments, a method is disclosed. The method is performed at a computer system that is in communication with one or more display generation components and one or more input devices, and comprises: detecting, via the one or more input devices, a first user input corresponding to a user request to display a health summary user interface; and in response to detecting the first user input, displaying, via the one or more display generation components, the health summary user interface, including: in accordance with a determination that a first set of health information has been received for a user of the computer system since the health summary user interface was last displayed, displaying, within a first region of the health summary user interface, a first set of information corresponding to a first type of health data based on the first set of health information; and in accordance with a determination that a second set of health information different from the first set of health information has been received for the user of the computer system since the health summary user interface was last displayed, displaying, within the first region of the health summary user interface, a second set of information corresponding to a second type of health data based on the second set of health information, wherein the second set of information is different from the first set of information, and the second type of health data is different from the first type of health data.

In some embodiments, a non-transitory computer-readable storage medium is disclosed. The non-transitory computer-readable storage medium stores one or more programs configured to be executed by one or more processors of a computer system that is in communication with one or more display generation components and one or more input devices, the one or more programs including instructions for: detecting, via the one or more input devices, a first user input corresponding to a user request to display a health summary user interface; and in response to detecting the first user input, displaying, via the one or more display generation components, the health summary user interface, including: in accordance with a determination that a first set of health information has been received for a user of the computer system since the health summary user interface was last displayed, displaying, within a first region of the health summary user interface, a first set of information corresponding to a first type of health data based on the first set of health information; and in accordance with a determination that a second set of health information different from the first set of health information has been received for the user of the computer system since the health summary user interface was last displayed, displaying, within the first region of the health summary user interface, a second set of information corresponding to a second type of health data based on the second set of health information, wherein the second set of information is different from the first set of information, and the second type of health data is different from the first type of health data.

In some embodiments, a transitory computer-readable storage medium is disclosed. The transitory computer-readable storage medium stores one or more programs configured to be executed by one or more processors of a computer system that is in communication with one or more display generation components and one or more input devices, the one or more programs including instructions for: detecting, via the one or more input devices, a first user input corresponding to a user request to display a health summary user interface; and in response to detecting the first user input, displaying, via the one or more display generation components, the health summary user interface, including: in accordance with a determination that a first set of health information has been received for a user of the computer system since the health summary user interface was last displayed, displaying, within a first region of the health summary user interface, a first set of information corresponding to a first type of health data based on the first set of health information; and in accordance with a determination that a second set of health information different from the first set of health information has been received for the user of the computer system since the health summary user interface was last displayed, displaying, within the first region of the health summary user interface, a second set of information corresponding to a second type of health data based on the second set of health information, wherein the second set of information is different from the first set of information, and the second type of health data is different from the first type of health data.

In some embodiments, a computer system is disclosed. The computer system is configured to communicate with one or more display generation components and one or more input devices, and comprises: one or more processors; and memory storing one or more programs configured to be executed by the one or more processors, the one or more programs including instructions for: detecting, via the one or more input devices, a first user input corresponding to a user request to display a health summary user interface; and in response to detecting the first user input, displaying, via the one or more display generation components, the health summary user interface, including: in accordance with a determination that a first set of health information has been received for a user of the computer system since the health summary user interface was last displayed, displaying, within a first region of the health summary user interface, a first set of information corresponding to a first type of health data based on the first set of health information; and in accordance with a determination that a second set of health information different from the first set of health information has been received for the user of the computer system since the health summary user interface was last displayed, displaying, within the first region of the health summary user interface, a second set of information corresponding to a second type of health data based on the second set of health information, wherein the second set of information is different from the first set of information, and the second type of health data is different from the first type of health data.

In some embodiments, a computer system is disclosed. The computer system is configured to communicate with one or more display generation components and one or more input devices, and comprises: means for detecting, via the one or more input devices, a first user input corresponding to a user request to display a health summary user interface; and means for, in response to detecting the first user input, displaying, via the one or more display generation components, the health summary user interface, including: in accordance with a determination that a first set of health information has been received for a user of the computer system since the health summary user interface was last displayed, displaying, within a first region of the health summary user interface, a first set of information corresponding to a first type of health data based on the first set of health information; and in accordance with a determination that a second set of health information different from the first set of health information has been received for the user of the computer system since the health summary user interface was last displayed, displaying, within the first region of the health summary user interface, a second set of information corresponding to a second type of health data based on the second set of health information, wherein the second set of information is different from the first set of information, and the second type of health data is different from the first type of health data.

In some embodiments, a computer program product is disclosed. The computer program product comprises one or more programs configured to be executed by one or more processors of a computer system that is in communication with one or more display generation components and one or more input devices, the one or more programs including instructions for: detecting, via the one or more input devices, a first user input corresponding to a user request to display a health summary user interface; and in response to detecting the first user input, displaying, via the one or more display generation components, the health summary user interface, including: in accordance with a determination that a first set of health information has been received for a user of the computer system since the health summary user interface was last displayed, displaying, within a first region of the health summary user interface, a first set of information corresponding to a first type of health data based on the first set of health information; and in accordance with a determination that a second set of health information different from the first set of health information has been received for the user of the computer system since the health summary user interface was last displayed, displaying, within the first region of the health summary user interface, a second set of information corresponding to a second type of health data based on the second set of health information, wherein the second set of information is different from the first set of information, and the second type of health data is different from the first type of health data.

In some embodiments, a method is disclosed. The method is performed at a computer system that is in communication with one or more display generation components and one or more input devices, and comprises: receiving first user health information corresponding to a user of the computer system; in response to receiving the first user health information, displaying, via the one or more display generation components, a first notification pertaining to the first user health information; while displaying the first notification, detecting, via the one or more input devices, a first user input corresponding to selection of the first notification; and in response to detecting the first user input, displaying, via the one or more display generation components, a first user interface, wherein the first user interface includes: additional user health information pertaining to the user of the computer system that was not displayed in the first notification; and a first prompt prompting the user of the computer system to ask a question.

In some embodiments, a non-transitory computer-readable storage medium is disclosed. The non-transitory computer-readable storage medium stores one or more programs configured to be executed by one or more processors of a computer system that is in communication with one or more display generation components and one or more input devices, the one or more programs including instructions for: receiving first user health information corresponding to a user of the computer system; in response to receiving the first user health information, displaying, via the one or more display generation components, a first notification pertaining to the first user health information; while displaying the first notification, detecting, via the one or more input devices, a first user input corresponding to selection of the first notification; and in response to detecting the first user input, displaying, via the one or more display generation components, a first user interface, wherein the first user interface includes: additional user health information pertaining to the user of the computer system that was not displayed in the first notification; and a first prompt prompting the user of the computer system to ask a question.

In some embodiments, a transitory computer-readable storage medium is disclosed. The transitory computer-readable storage medium stores one or more programs configured to be executed by one or more processors of a computer system that is in communication with one or more display generation components and one or more input devices, the one or more programs including instructions for: receiving first user health information corresponding to a user of the computer system; in response to receiving the first user health information, displaying, via the one or more display generation components, a first notification pertaining to the first user health information; while displaying the first notification, detecting, via the one or more input devices, a first user input corresponding to selection of the first notification; and in response to detecting the first user input, displaying, via the one or more display generation components, a first user interface, wherein the first user interface includes: additional user health information pertaining to the user of the computer system that was not displayed in the first notification; and a first prompt prompting the user of the computer system to ask a question.

In some embodiments, a computer system is disclosed. The computer system is configured to communicate with one or more display generation components and one or more input devices, and comprises: one or more processors; and memory storing one or more programs configured to be executed by the one or more processors, the one or more programs including instructions for: receiving first user health information corresponding to a user of the computer system; in response to receiving the first user health information, displaying, via the one or more display generation components, a first notification pertaining to the first user health information; while displaying the first notification, detecting, via the one or more input devices, a first user input corresponding to selection of the first notification; and in response to detecting the first user input, displaying, via the one or more display generation components, a first user interface, wherein the first user interface includes: additional user health information pertaining to the user of the computer system that was not displayed in the first notification; and a first prompt prompting the user of the computer system to ask a question.

In some embodiments, a computer system is disclosed. The computer system is configured to communicate with one or more display generation components and one or more input devices, and comprises: receiving first user health information corresponding to a user of the computer system; in response to receiving the first user health information, displaying, via the one or more display generation components, a first notification pertaining to the first user health information; while displaying the first notification, detecting, via the one or more input devices, a first user input corresponding to selection of the first notification; and in response to detecting the first user input, displaying, via the one or more display generation components, a first user interface, wherein the first user interface includes: additional user health information pertaining to the user of the computer system that was not displayed in the first notification; and a first prompt prompting the user of the computer system to ask a question.

In some embodiments, a computer program product is disclosed. The computer program product comprises one or more programs configured to be executed by one or more processors of a computer system that is in communication with one or more display generation components and one or more input devices, the one or more programs including instructions for: receiving first user health information corresponding to a user of the computer system; in response to receiving the first user health information, displaying, via the one or more display generation components, a first notification pertaining to the first user health information; while displaying the first notification, detecting, via the one or more input devices, a first user input corresponding to selection of the first notification; and in response to detecting the first user input, displaying, via the one or more display generation components, a first user interface, wherein the first user interface includes: additional user health information pertaining to the user of the computer system that was not displayed in the first notification; and a first prompt prompting the user of the computer system to ask a question.

Executable instructions for performing these functions are, optionally, included in a non-transitory computer-readable storage medium or other computer program product configured for execution by one or more processors. Executable instructions for performing these functions are, optionally, included in a transitory computer-readable storage medium or other computer program product configured for execution by one or more processors.

Thus, devices are provided with faster, more efficient methods and interfaces for tracking and presenting user health information, thereby increasing the effectiveness, efficiency, and user satisfaction with such devices. Such methods and interfaces may complement or replace other methods for tracking and presenting user health information.

### DESCRIPTION OF THE FIGURES

For a better understanding of the various described embodiments, reference should be made to the Description of Embodiments below, in conjunction with the following drawings in which like reference numerals refer to corresponding parts throughout the figures.

FIG. 1A is a block diagram illustrating a portable multifunction device with a touch-sensitive display in accordance with some embodiments.

FIG. 1B is a block diagram illustrating exemplary components for event handling in accordance with some embodiments.

FIG. 2 illustrates a portable multifunction device having a touch screen in accordance with some embodiments.

FIG. 3A is a block diagram of an exemplary multifunction device with a display and a touch-sensitive surface in accordance with some embodiments.

FIGS. 3B-3G illustrate the use of Application Programming Interfaces (APIs) to perform operations.

FIG. 4A illustrates an exemplary user interface for a menu of applications on a portable multifunction device in accordance with some embodiments.

FIG. 4B illustrates an exemplary user interface for a multifunction device with a touch-sensitive surface that is separate from the display in accordance with some embodiments.

FIG. 5A illustrates a personal electronic device in accordance with some embodiments.

FIG. 5B is a block diagram illustrating a personal electronic device in accordance with some embodiments.

FIGS. 6A-6GG illustrate exemplary user interfaces for tracking and providing user health information, in accordance with some embodiments.

FIG. 7 illustrates a flow diagram depicting a method for providing user health information, in accordance with some embodiments.

FIGS. 8A-8H illustrate exemplary user interfaces for tracking and providing user health information, in accordance with some embodiments.

FIG. 9 illustrates a flow diagram depicting a method for providing user health information, in accordance with some embodiments.

### DESCRIPTION OF EMBODIMENTS

The following description sets forth exemplary methods, parameters, and the like. It should be recognized, however, that such description is not intended as a limitation on the scope of the present disclosure but is instead provided as a description of exemplary embodiments.

There is a need for electronic devices that provide efficient methods and interfaces for tracking and presenting user health information. Such techniques can reduce the cognitive burden on a user who tracks and/or accesses health data, thereby enhancing productivity. Further, such techniques can reduce processor and battery power otherwise wasted on redundant user inputs.

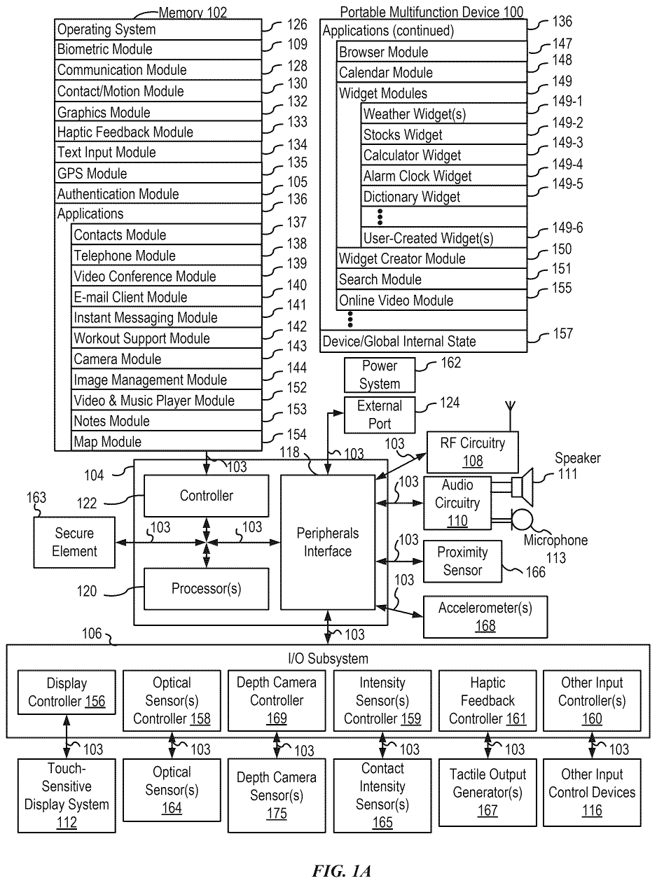

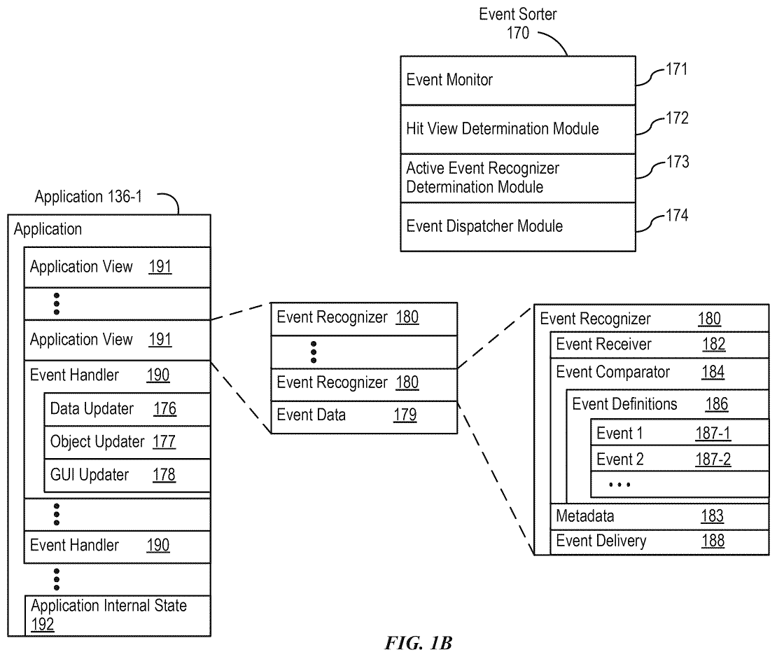

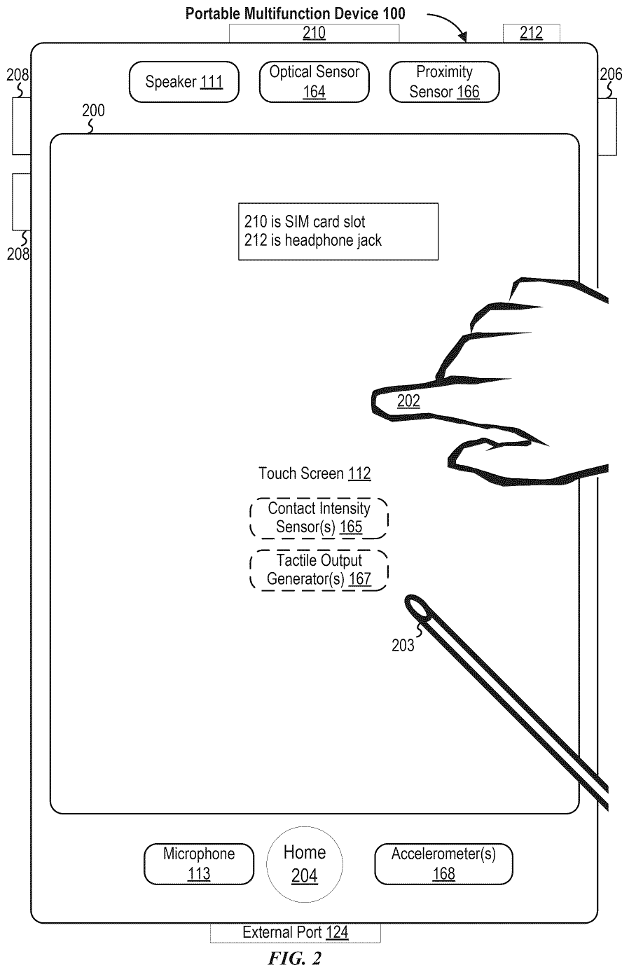

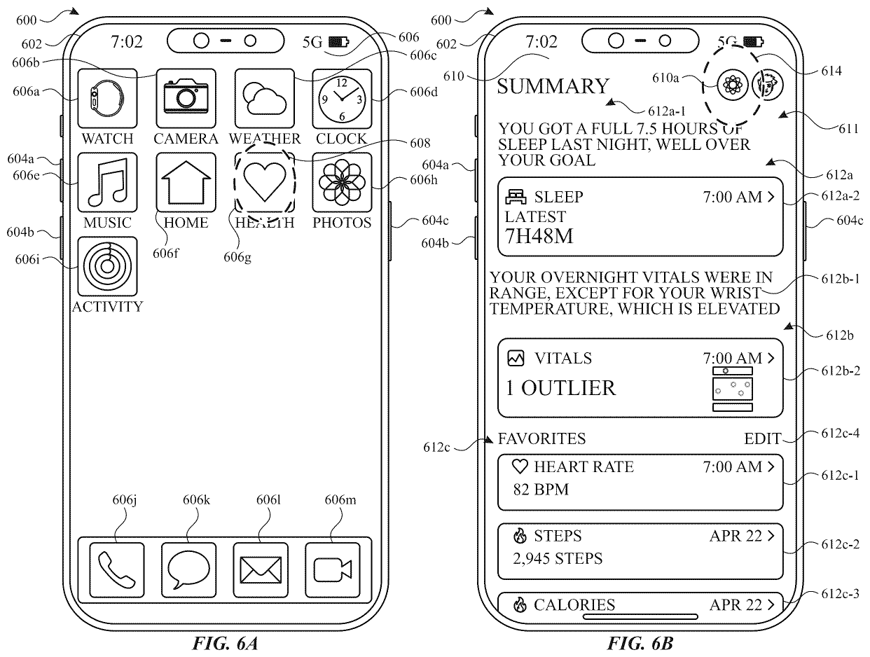

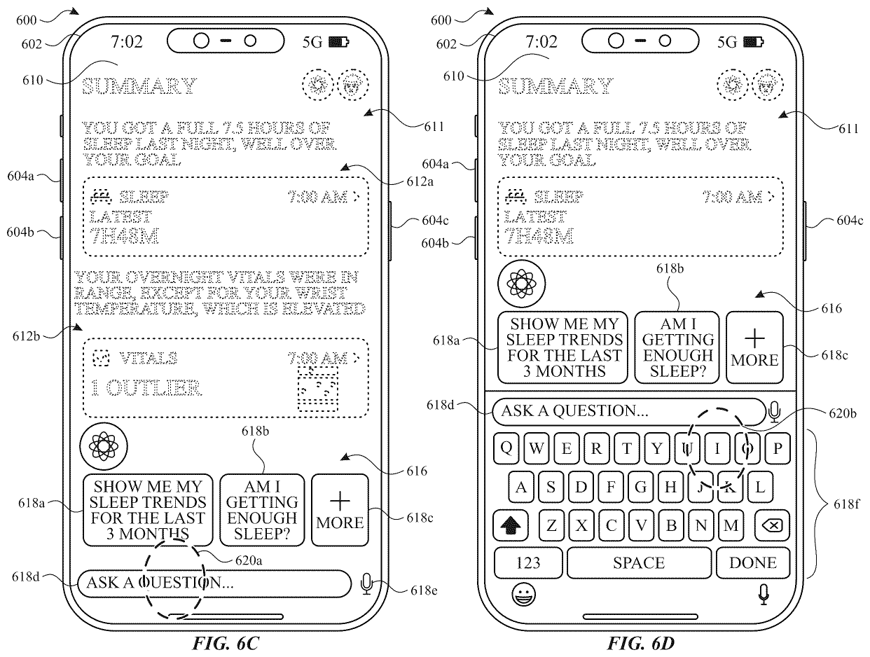

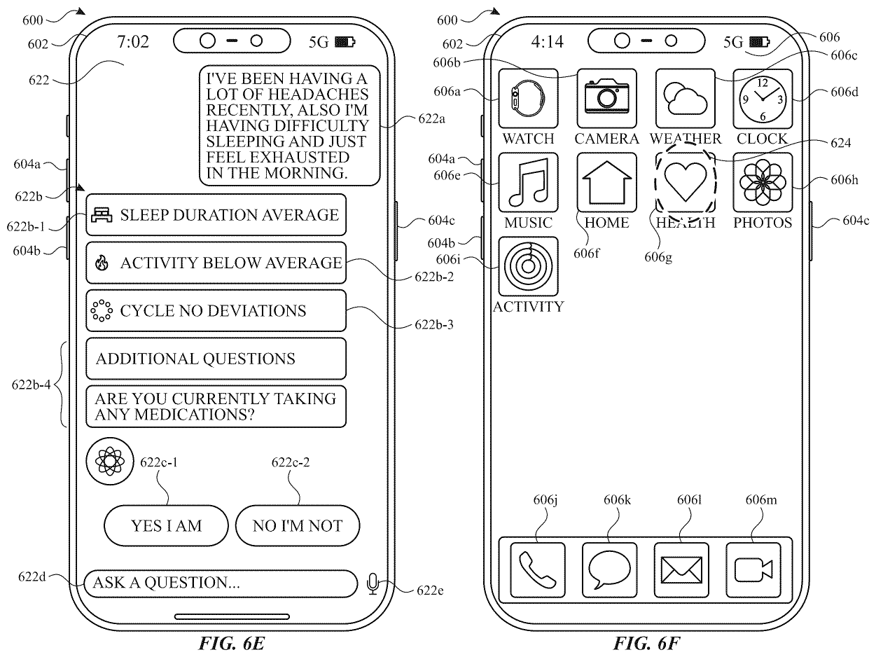

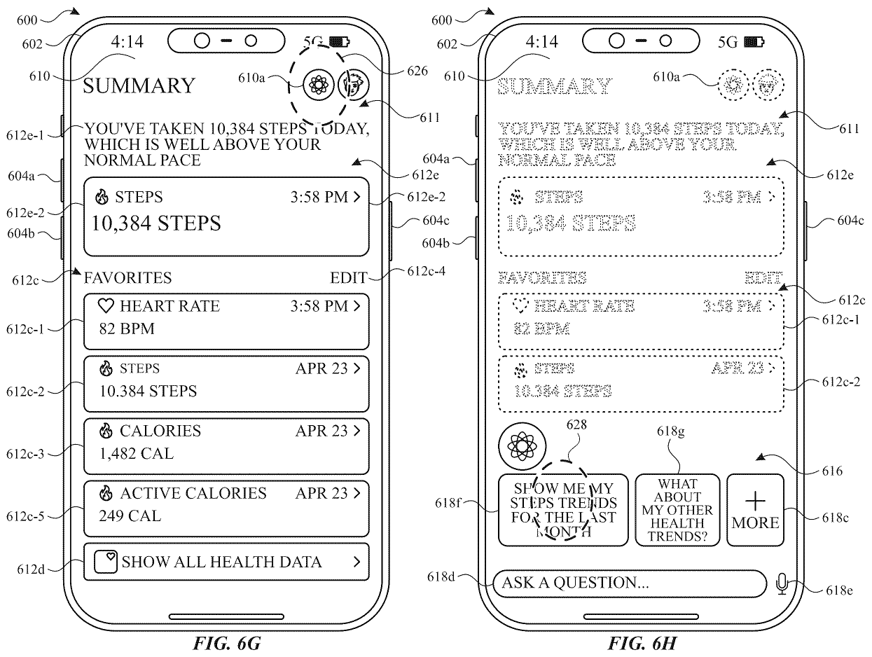

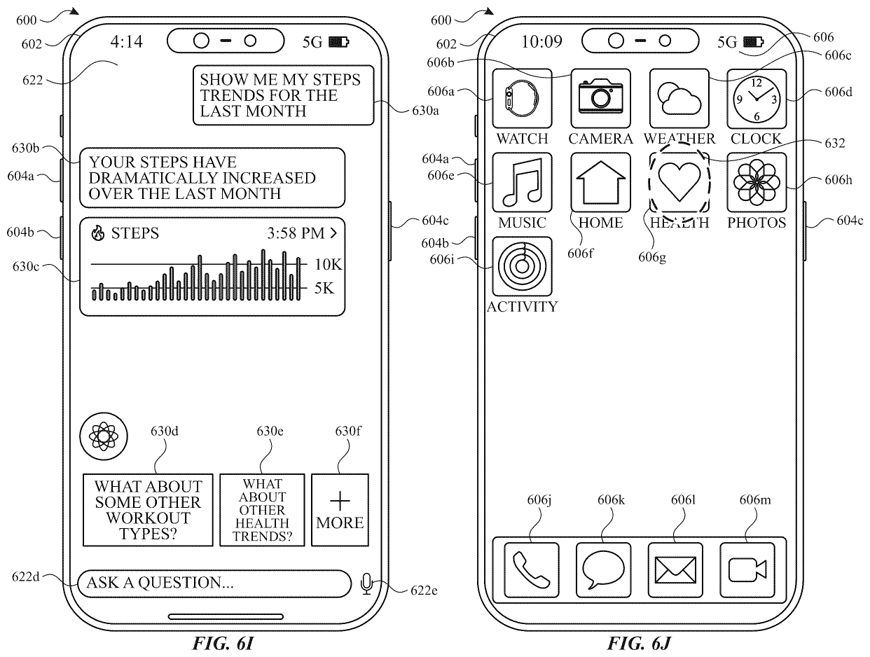

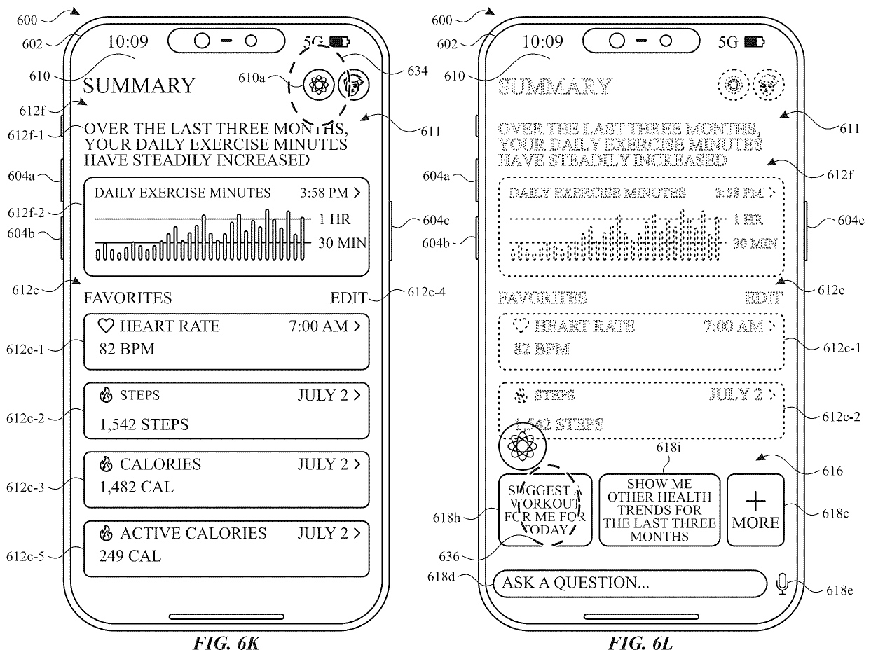

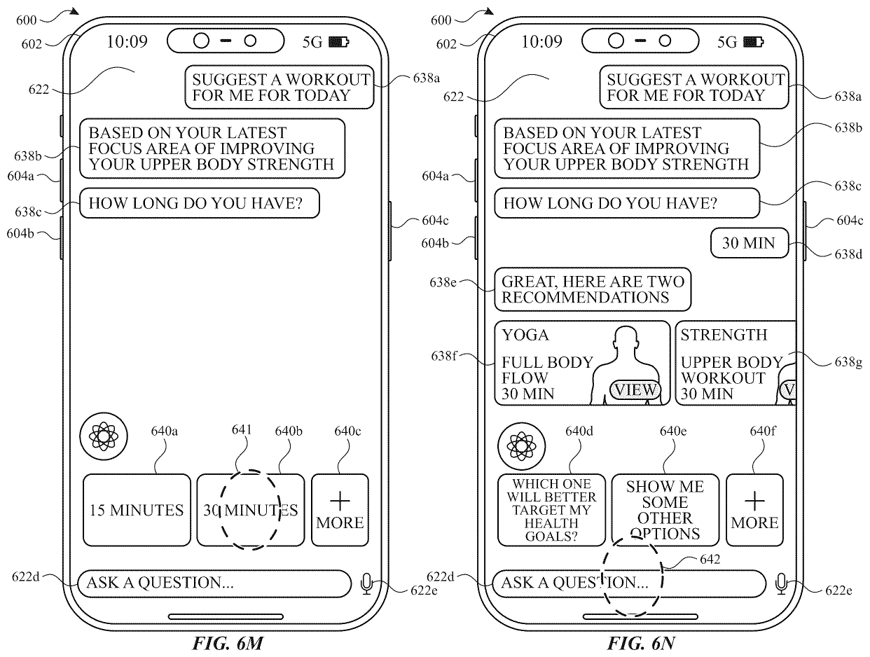

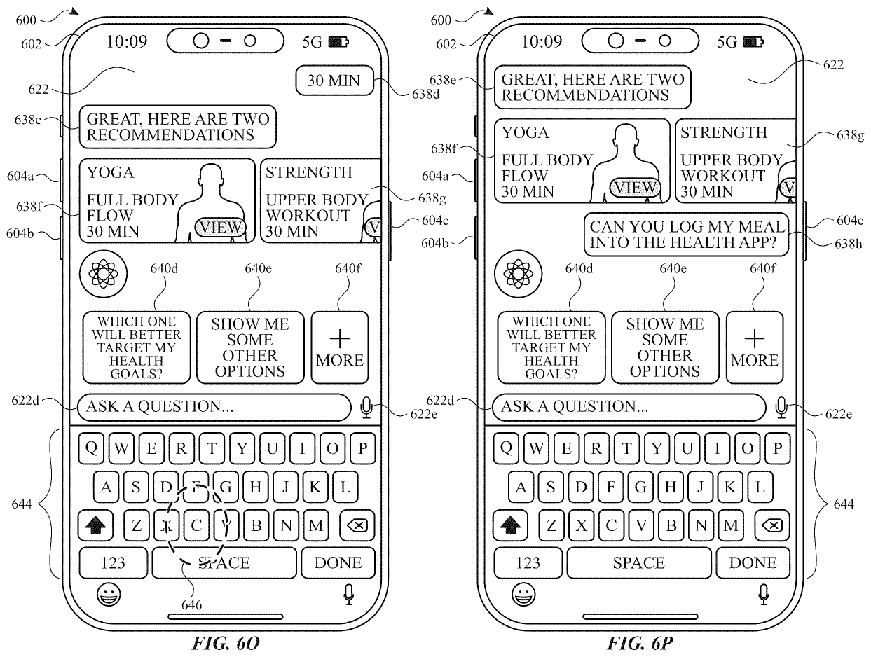

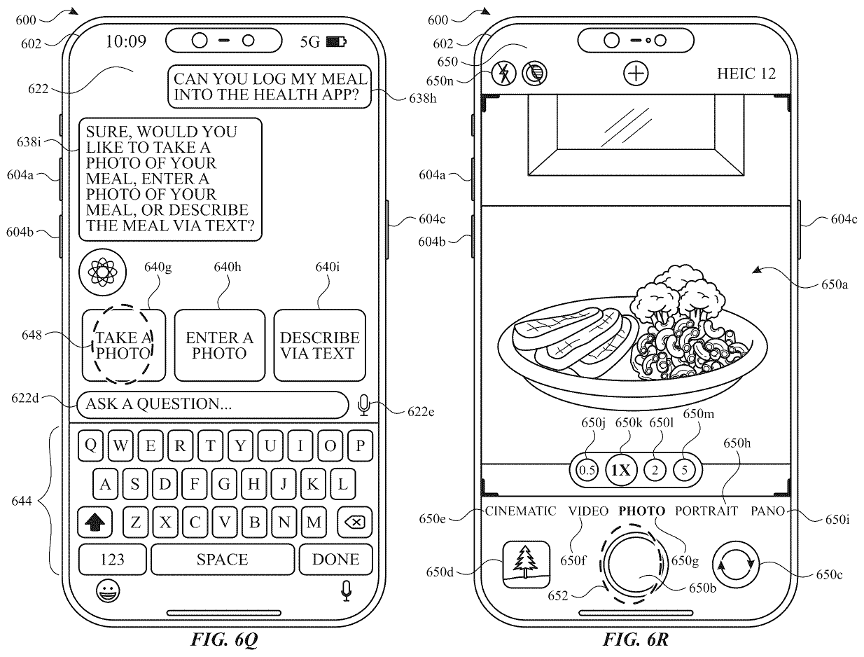

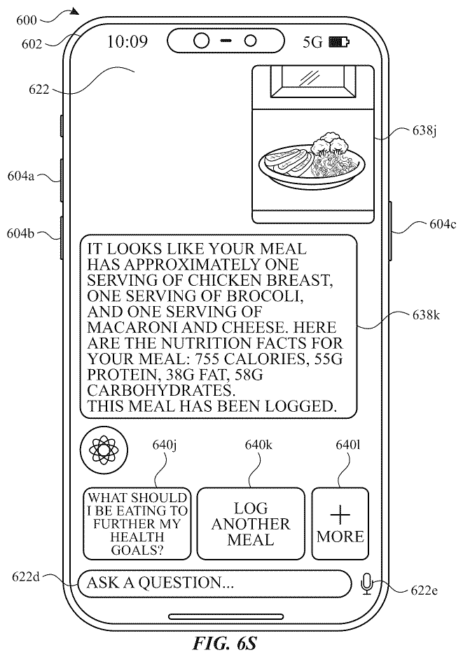

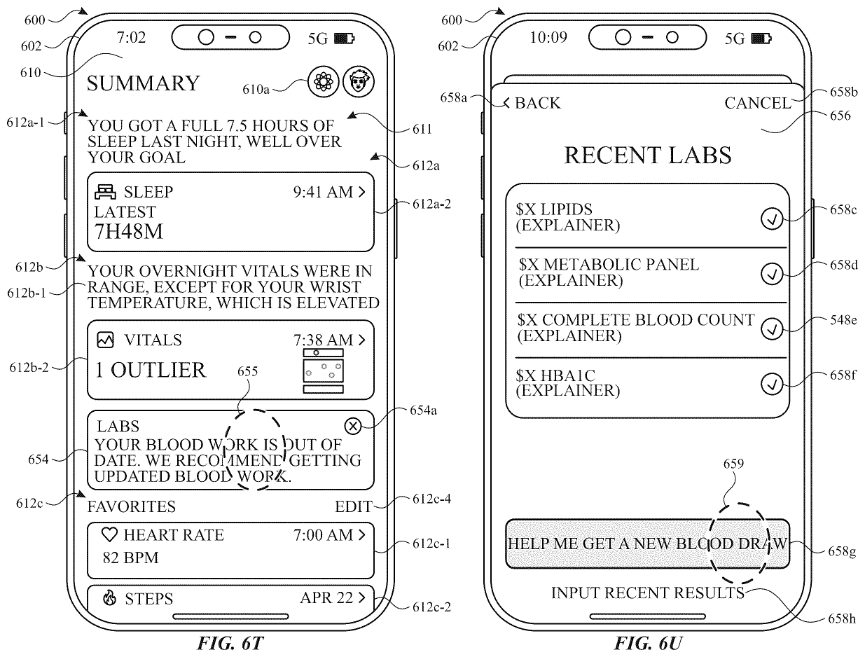

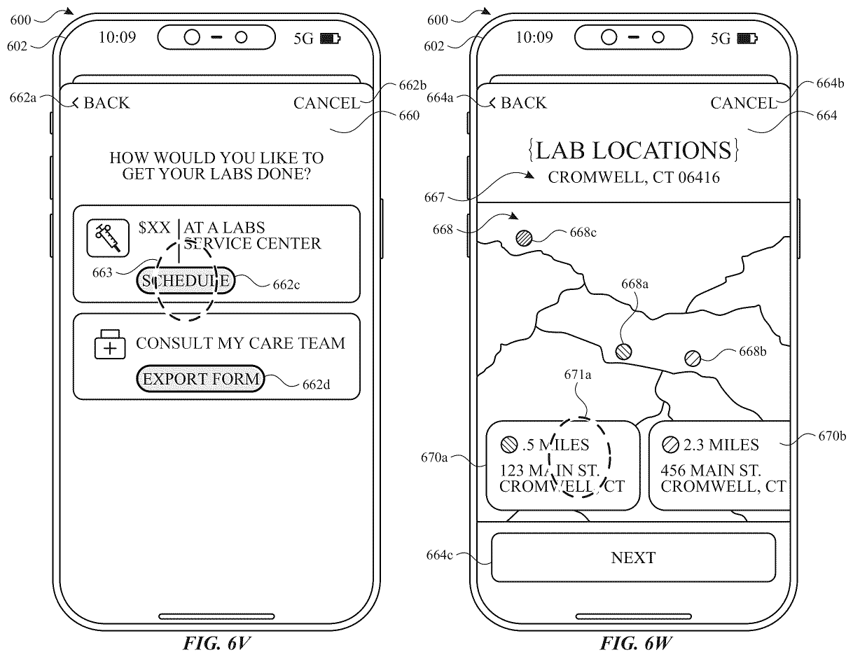

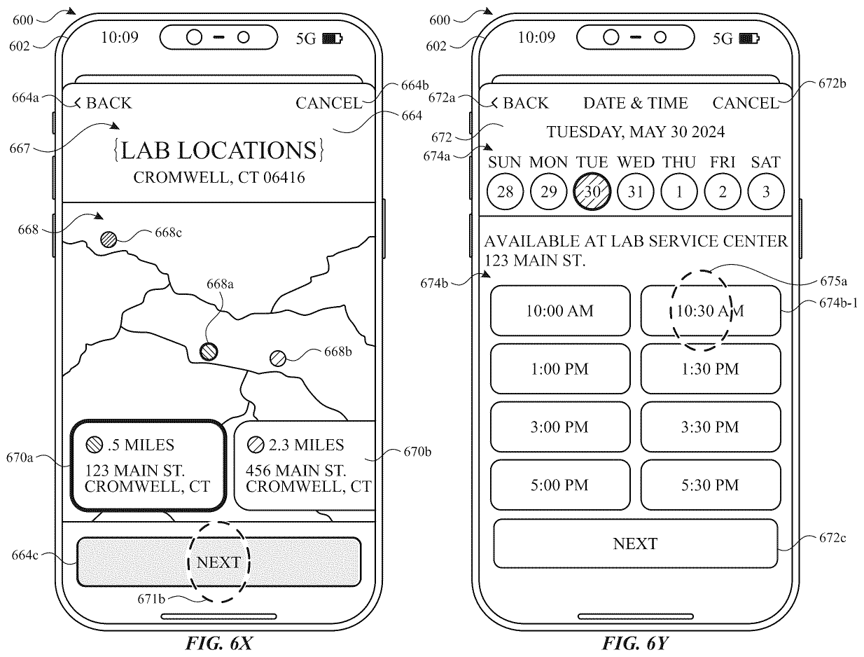

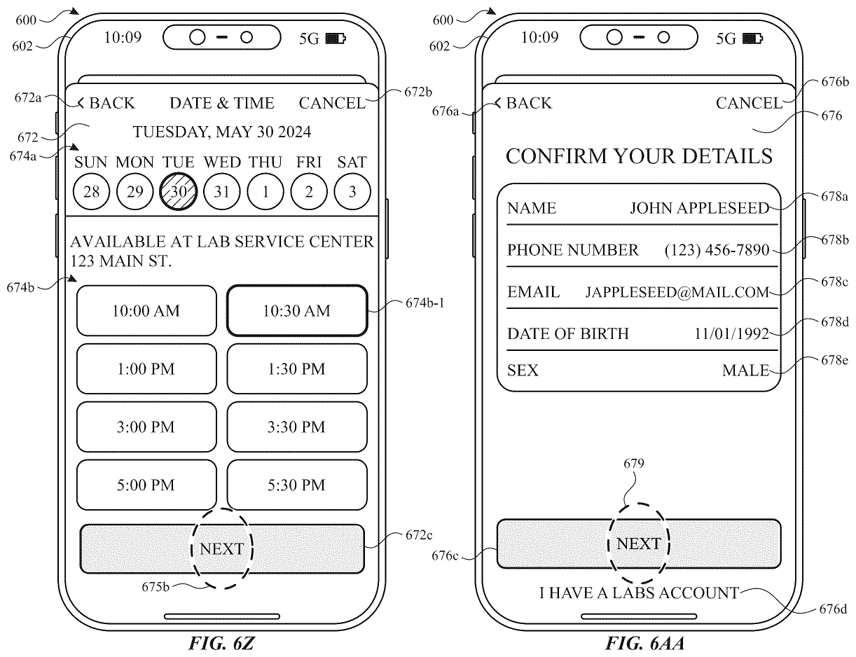

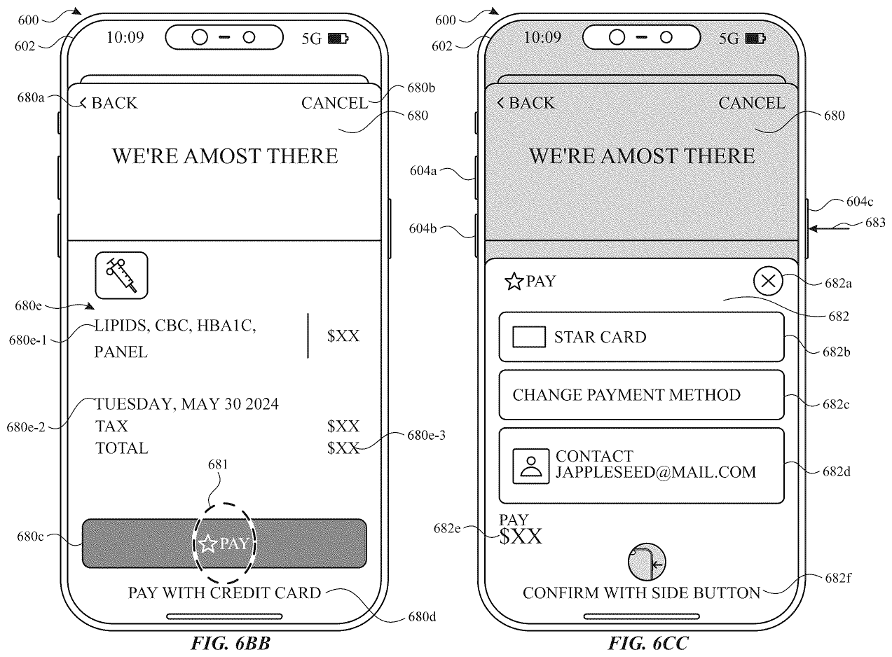

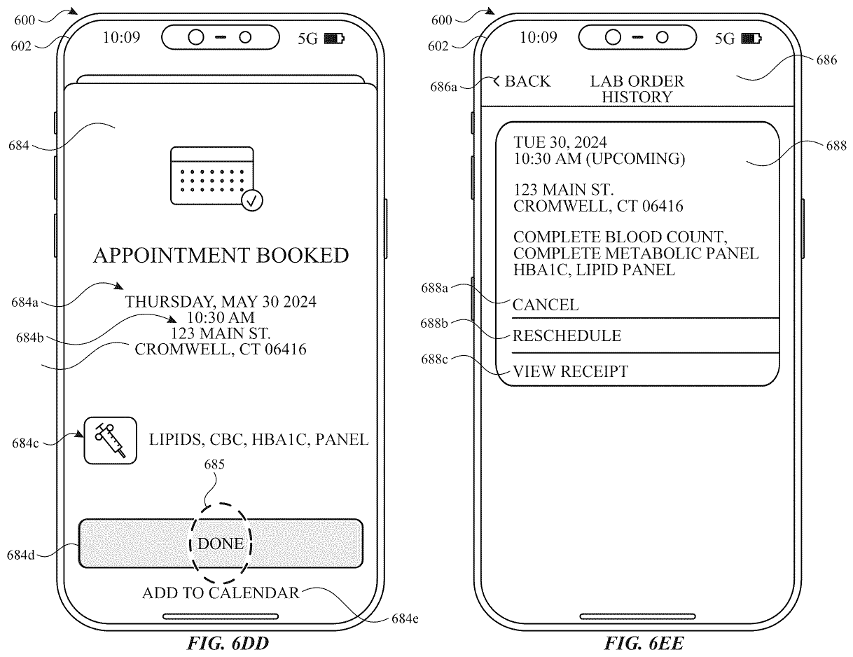

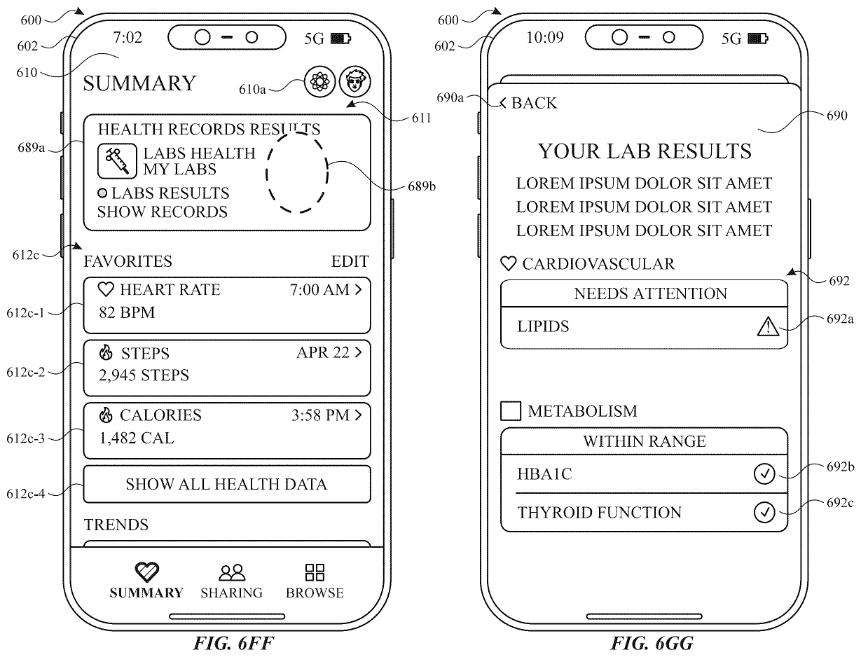

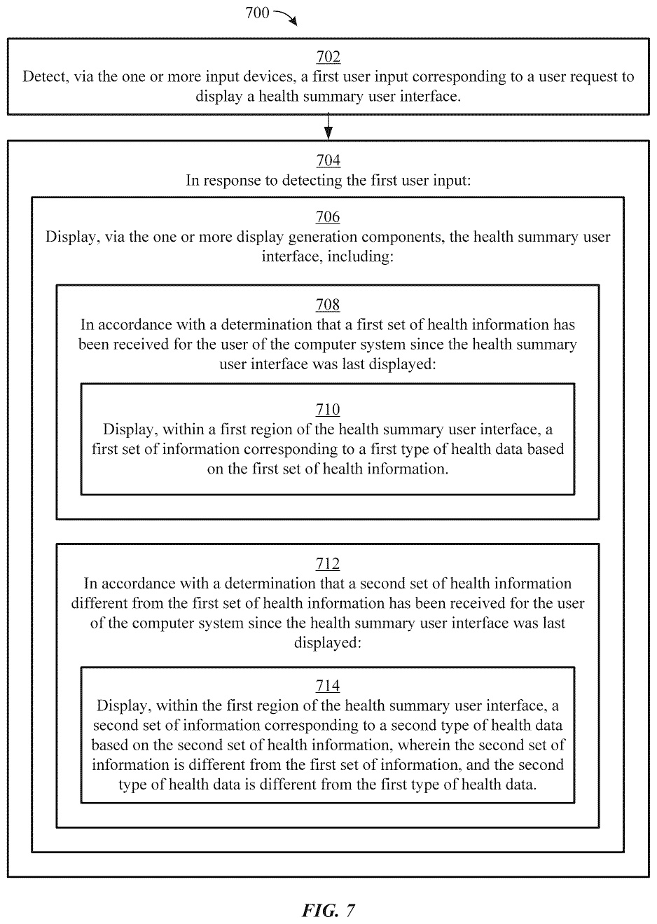

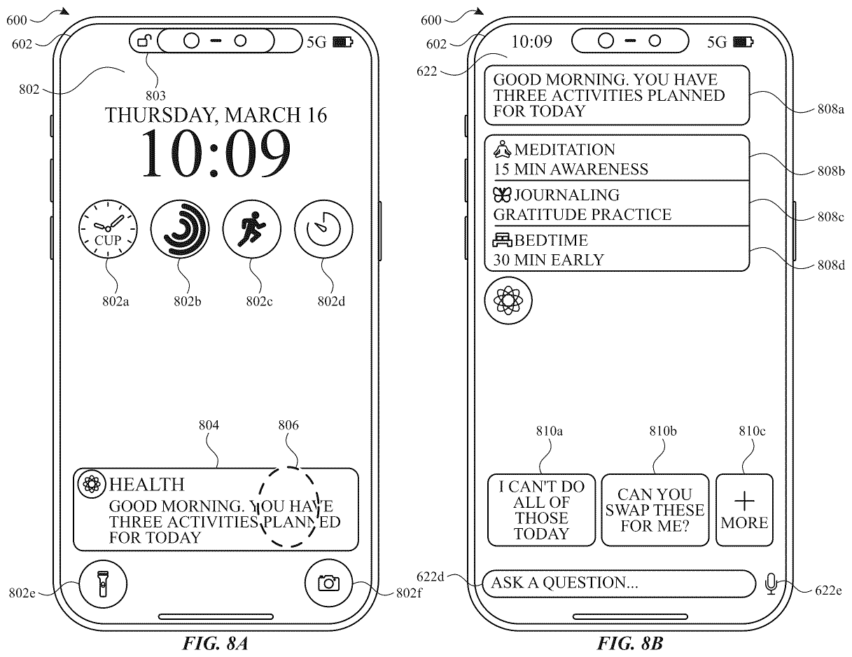

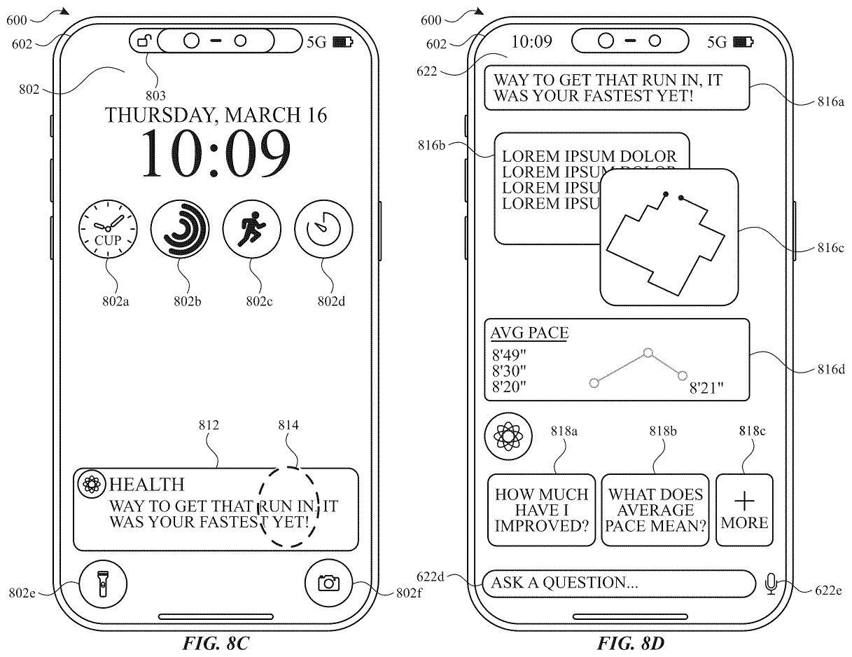

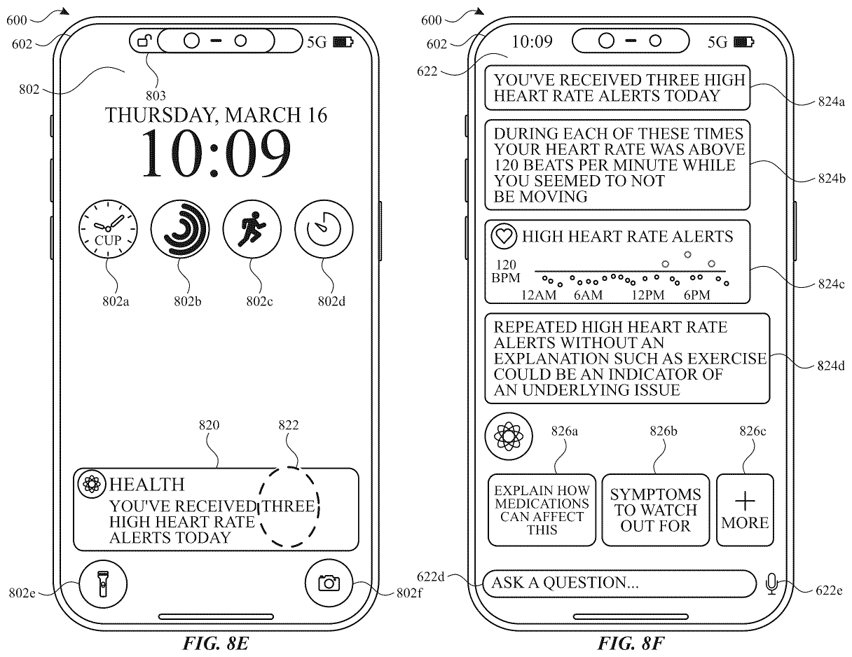

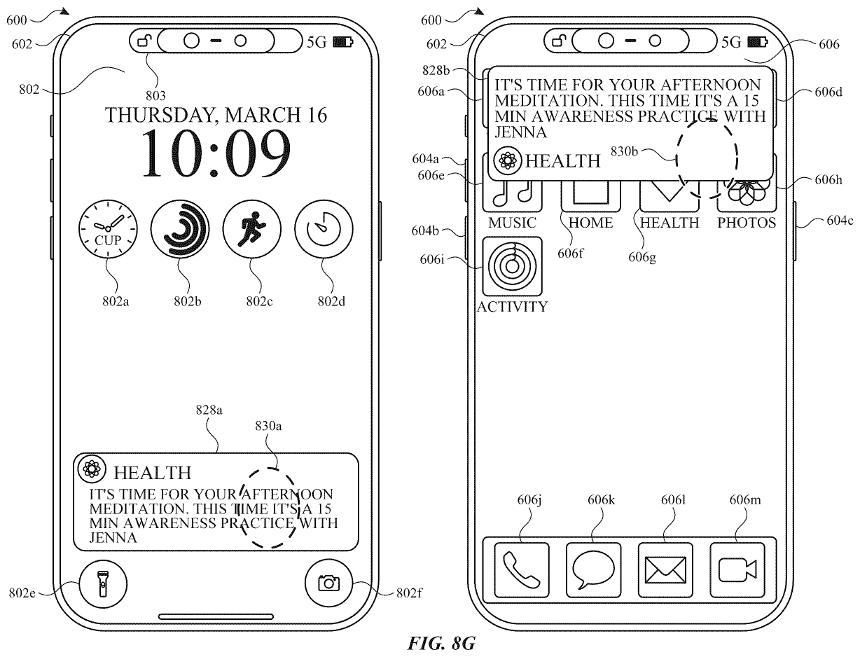

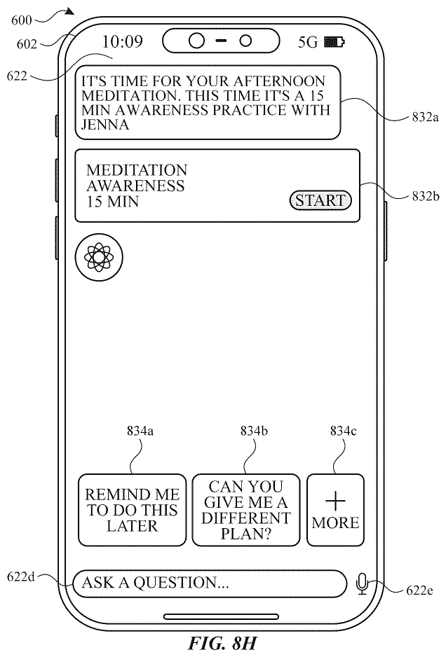

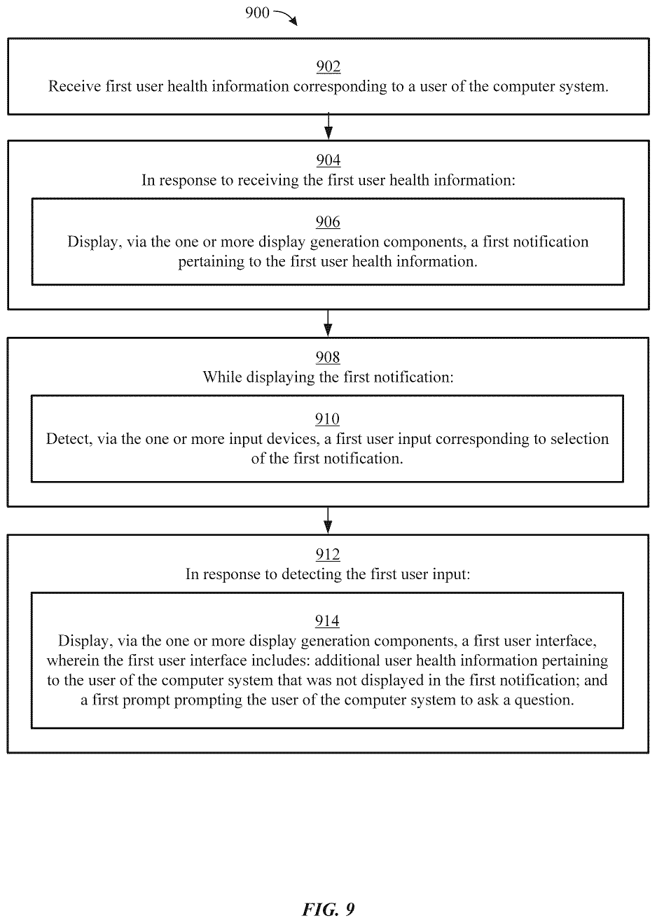

Below, FIGS. 1A-1B, 2, 3A-3G, 4A-4B, and 5A-5B provide a description of exemplary devices for performing the techniques for managing event notifications. FIGS. 6A-6GG illustrate exemplary user interfaces for tracking and providing user health information. FIG. 7 is a flow diagram illustrating methods of providing user health information in accordance with some embodiments. The user interfaces in FIGS. 6A-6GG are used to illustrate the processes described below, including the processes in FIG. 7. FIGS. 8A-8H illustrate exemplary user interfaces for tracking and providing user health information. FIG. 9 is a flow diagram illustrating methods of providing user health information in accordance with some embodiments. The user interfaces in FIGS. 8A-8H are used to illustrate the processes described below, including the processes in FIG. 9.

The processes described below enhance the operability of the devices and make the user-device interfaces more efficient (e.g., by helping the user to provide proper inputs and reducing user mistakes when operating/interacting with the device) through various techniques, including by providing improved visual feedback to the user, reducing the number of inputs needed to perform an operation, providing additional control options without cluttering the user interface with additional displayed controls, performing an operation when a set of conditions has been met without requiring further user input, and/or additional techniques. These techniques also reduce power usage and improve battery life of the device by enabling the user to use the device more quickly and efficiently.

In addition, in methods described herein where one or more steps are contingent upon one or more conditions having been met, it should be understood that the described method can be repeated in multiple repetitions so that over the course of the repetitions all of the conditions upon which steps in the method are contingent have been met in different repetitions of the method. For example, if a method requires performing a first step if a condition is satisfied, and a second step if the condition is not satisfied, then a person of ordinary skill would appreciate that the claimed steps are repeated until the condition has been both satisfied and not satisfied, in no particular order. Thus, a method described with one or more steps that are contingent upon one or more conditions having been met could be rewritten as a method that is repeated until each of the conditions described in the method has been met. This, however, is not required of system or computer readable medium claims where the system or computer readable medium contains instructions for performing the contingent operations based on the satisfaction of the corresponding one or more conditions and thus is capable of determining whether the contingency has or has not been satisfied without explicitly repeating steps of a method until all of the conditions upon which steps in the method are contingent have been met. A person having ordinary skill in the art would also understand that, similar to a method with contingent steps, a system or computer readable storage medium can repeat the steps of a method as many times as are needed to ensure that all of the contingent steps have been performed.

Although the following description uses terms “first,” “second,” etc. to describe various elements, these elements should not be limited by the terms. In some embodiments, these terms are used to distinguish one element from another. For example, a first touch could be termed a second touch, and, similarly, a second touch could be termed a first touch, without departing from the scope of the various described embodiments. In some embodiments, the first touch and the second touch are two separate references to the same touch. In some embodiments, the first touch and the second touch are both touches, but they are not the same touch.

As used herein, the phrase “one or more of A and/or B” is construed to include all combinations of A and B, including, but not limited to: A individually without B; B individually without A; as well as a combination of A and B. The phrase “one or more of A, B, and/or C” is construed to include all combinations of A, B, and C, including, but not limited to: A individually without B and C; B individually without A and C; C individually without A and B; as well as any combinations of A, B, and/or C (e.g., A and B without C; A and C without B; B and C without A; and/or A, B, and C). Additionally, as used herein, the phrase “selected from the group consisting of A, B, C, and a combination thereof” and the phrase “at least one of A, B, and C” shall be construed to have the same meaning as the phrase “one or more of A, B, and/or C” as defined above. As used herein, the phrase “at least one of A, B, or C” and “one or more of A, B, or C” shall be construed to have the same meaning as the phrase “one or more of A, B, and/or C” as defined above. As used herein, the phrase “a combination including all of A, B, and C” is construed to include a combination of all the elements listed (e.g., a combination of A, B, and C).

The terminology used in the description of the various described embodiments herein is for the purpose of describing particular embodiments only and is not intended to be limiting.

As used in the description of the various described embodiments and the appended claims, the singular forms “a,” “an,” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “includes,” “including,” “comprises,” and/or “comprising,” when used in this specification, specify the presence of stated features, integers, steps, operations, elements, and/or components, but do not preclude the presence or addition of one or more other features, integers, steps, operations, elements, components, and/or groups thereof.

The term “if” is, optionally, construed to mean “when” or “upon” or “in response to determining” or “in response to detecting,” depending on the context. Similarly, the phrase “if it is determined” or “if [a stated condition or event] is detected” is, optionally, construed to mean “upon determining” or “in response to determining” or “upon detecting [the stated condition or event]” or “in response to detecting [the stated condition or event],”depending on the context.

Embodiments of electronic devices, user interfaces for such devices, and associated processes for using such devices are described. In some embodiments, the device is a portable communications device, such as a mobile telephone, that also contains other functions, such as PDA and/or music player functions. Exemplary embodiments of portable multifunction devices include, without limitation, the iPhone®, iPod Touch®, and iPad® devices from Apple Inc. of Cupertino, California. Other portable electronic devices, such as laptops or tablet computers with touch-sensitive surfaces (e.g., touch screen displays and/or touchpads), are, optionally, used. It should also be understood that, in some embodiments, the device is not a portable communications device, but is a desktop computer with a touch-sensitive surface (e.g., a touch screen display and/or a touchpad). In some embodiments, the electronic device is a computer system that is in communication (e.g., via wireless communication, via wired communication) with a display generation component (e.g., a display device such as a head-mounted display (HMD), a display, a projector, a touch-sensitive display, or other device or component that presents visual content to a user, for example on or in the display generation component itself or produced from the display generation component and visible elsewhere). The display generation component is configured to provide visual output, such as display via a CRT display, display via an LED display, or display via image projection. In some embodiments, the display generation component is integrated with the computer system. In some embodiments, the display generation component is separate from the computer system. As used herein, “displaying” content includes causing to display the content (e.g., video data rendered or decoded by display controller 156) by transmitting, via a wired or wireless connection, data (e.g., image data or video data) to an integrated or external display generation component to visually produce the content.

In the discussion that follows, an electronic device that includes a display and a touch-sensitive surface is described. It should be understood, however, that the electronic device optionally includes one or more other physical user-interface devices, such as a physical keyboard, a mouse, and/or a joystick.

The device typically supports a variety of applications, such as one or more of the following: a drawing application, a presentation application, a word processing application, a website creation application, a disk authoring application, a spreadsheet application, a gaming application, a telephone application, a video conferencing application, an e-mail application, an instant messaging application, a workout support application, a photo management application, a digital camera application, a digital video camera application, a web browsing application, a digital music player application, and/or a digital video player application.

The various applications that are executed on the device optionally use at least one common physical user-interface device, such as the touch-sensitive surface. One or more functions of the touch-sensitive surface as well as corresponding information displayed on the device are, optionally, adjusted and/or varied from one application to the next and/or within a respective application. In this way, a common physical architecture (such as the touch-sensitive surface) of the device optionally supports the variety of applications with user interfaces that are intuitive and transparent to the user.

Attention is now directed toward embodiments of portable devices with touch-sensitive displays. FIG. 1A is a block diagram illustrating portable multifunction device 100 with touch-sensitive display system 112 in accordance with some embodiments. Touch-sensitive display 112 is sometimes called a “touch screen” for convenience and is sometimes known as or called a “touch-sensitive display system.” Device 100 includes memory 102 (which optionally includes one or more computer-readable storage media), memory controller 122, one or more processing units (CPUs) 120, peripherals interface 118, RF circuitry 108, audio circuitry 110, speaker 111, microphone 113, input/output (I/O) subsystem 106, other input control devices 116, and external port 124. Device 100 optionally includes one or more optical sensors 164. Device 100 optionally includes one or more contact intensity sensors 165 for detecting intensity of contacts on device 100 (e.g., a touch-sensitive surface such as touch-sensitive display system 112 of device 100). Device 100 optionally includes one or more tactile output generators 167 for generating tactile outputs on device 100 (e.g., generating tactile outputs on a touch-sensitive surface such as touch-sensitive display system 112 of device 100 or touchpad 355 of device 300). These components optionally communicate over one or more communication buses or signal lines 103.

As used in the specification and claims, the term “intensity” of a contact on a touch-sensitive surface refers to the force or pressure (force per unit area) of a contact (e.g., a finger contact) on the touch-sensitive surface, or to a substitute (proxy) for the force or pressure of a contact on the touch-sensitive surface. The intensity of a contact has a range of values that includes at least four distinct values and more typically includes hundreds of distinct values (e.g., at least 256). Intensity of a contact is, optionally, determined (or measured) using various approaches and various sensors or combinations of sensors. For example, one or more force sensors underneath or adjacent to the touch-sensitive surface are, optionally, used to measure force at various points on the touch-sensitive surface. In some implementations, force measurements from multiple force sensors are combined (e.g., a weighted average) to determine an estimated force of a contact. Similarly, a pressure-sensitive tip of a stylus is, optionally, used to determine a pressure of the stylus on the touch-sensitive surface. Alternatively, the size of the contact area detected on the touch-sensitive surface and/or changes thereto, the capacitance of the touch-sensitive surface proximate to the contact and/or changes thereto, and/or the resistance of the touch-sensitive surface proximate to the contact and/or changes thereto are, optionally, used as a substitute for the force or pressure of the contact on the touch-sensitive surface. In some implementations, the substitute measurements for contact force or pressure are used directly to determine whether an intensity threshold has been exceeded (e.g., the intensity threshold is described in units corresponding to the substitute measurements). In some implementations, the substitute measurements for contact force or pressure are converted to an estimated force or pressure, and the estimated force or pressure is used to determine whether an intensity threshold has been exceeded (e.g., the intensity threshold is a pressure threshold measured in units of pressure). Using the intensity of a contact as an attribute of a user input allows for user access to additional device functionality that may otherwise not be accessible by the user on a reduced-size device with limited real estate for displaying affordances (e.g., on a touch-sensitive display) and/or receiving user input (e.g., via a touch-sensitive display, a touch-sensitive surface, or a physical/mechanical control such as a knob or a button).

As used in the specification and claims, the term “tactile output” refers to physical displacement of a device relative to a previous position of the device, physical displacement of a component (e.g., a touch-sensitive surface) of a device relative to another component (e.g., housing) of the device, or displacement of the component relative to a center of mass of the device that will be detected by a user with the user's sense of touch. For example, in situations where the device or the component of the device is in contact with a surface of a user that is sensitive to touch (e.g., a finger, palm, or other part of a user's hand), the tactile output generated by the physical displacement will be interpreted by the user as a tactile sensation corresponding to a perceived change in physical characteristics of the device or the component of the device. For example, movement of a touch-sensitive surface (e.g., a touch-sensitive display or trackpad) is, optionally, interpreted by the user as a “down click” or “up click” of a physical actuator button. In some cases, a user will feel a tactile sensation such as an “down click” or “up click” even when there is no movement of a physical actuator button associated with the touch-sensitive surface that is physically pressed (e.g., displaced) by the user's movements. As another example, movement of the touch-sensitive surface is, optionally, interpreted or sensed by the user as “roughness” of the touch-sensitive surface, even when there is no change in smoothness of the touch-sensitive surface. While such interpretations of touch by a user will be subject to the individualized sensory perceptions of the user, there are many sensory perceptions of touch that are common to a large majority of users. Thus, when a tactile output is described as corresponding to a particular sensory perception of a user (e.g., an “up click,” a “down click,” “roughness”), unless otherwise stated, the generated tactile output corresponds to physical displacement of the device or a component thereof that will generate the described sensory perception for a typical (or average) user.

It should be appreciated that device 100 is only one example of a portable multifunction device, and that device 100 optionally has more or fewer components than shown, optionally combines two or more components, or optionally has a different configuration or arrangement of the components. The various components shown in FIG. 1A are implemented in hardware, software, or a combination of both hardware and software, including one or more signal processing and/or application-specific integrated circuits.

Memory 102 optionally includes high-speed random access memory and optionally also includes non-volatile memory, such as one or more magnetic disk storage devices, flash memory devices, or other non-volatile solid-state memory devices. Memory controller 122 optionally controls access to memory 102 by other components of device 100.

Peripherals interface 118 can be used to couple input and output peripherals of the device to CPU 120 and memory 102. The one or more processors 120 run or execute various software programs (such as computer programs (e.g., including instructions)) and/or sets of instructions stored in memory 102 to perform various functions for device 100 and to process data. In some embodiments, peripherals interface 118, CPU 120, and memory controller 122 are, optionally, implemented on a single chip, such as chip 104. In some other embodiments, they are, optionally, implemented on separate chips.

RF (radio frequency) circuitry 108 receives and sends RF signals, also called electromagnetic signals. RF circuitry 108 converts electrical signals to/from electromagnetic signals and communicates with communications networks and other communications devices via the electromagnetic signals. RF circuitry 108 optionally includes well-known circuitry for performing these functions, including but not limited to an antenna system, an RF transceiver, one or more amplifiers, a tuner, one or more oscillators, a digital signal processor, a CODEC chipset, a subscriber identity module (SIM) card, memory, and so forth. RF circuitry 108 optionally communicates with networks, such as the Internet, also referred to as the World Wide Web (WWW), an intranet and/or a wireless network, such as a cellular telephone network, a wireless local area network (LAN) and/or a metropolitan area network (MAN), and other devices by wireless communication. The RF circuitry 108 optionally includes well-known circuitry for detecting near field communication (NFC) fields, such as by a short-range communication radio. The wireless communication optionally uses any of a plurality of communications standards, protocols, and technologies, including but not limited to Global System for Mobile Communications (GSM), Enhanced Data GSM Environment (EDGE), high-speed downlink packet access (HSDPA), high-speed uplink packet access (HSUPA), Evolution, Data-Only (EV-DO), HSPA, HSPA+, Dual-Cell HSPA (DC-HSPDA), long term evolution (LTE), near field communication (NFC), wideband code division multiple access (W-CDMA), code division multiple access (CDMA), time division multiple access (TDMA), Bluetooth, Bluetooth Low Energy (BTLE), Wireless Fidelity (Wi-Fi) (e.g., IEEE 802.11a, IEEE 802.11b, IEEE 802.11g, IEEE 802.11n, and/or IEEE 802.11ac), voice over Internet Protocol (VoIP), Wi-MAX, a protocol for e-mail (e.g., Internet message access protocol (IMAP) and/or post office protocol (POP)), instant messaging (e.g., extensible messaging and presence protocol (XMPP), Session Initiation Protocol for Instant Messaging and Presence Leveraging Extensions (SIMPLE), Instant Messaging and Presence Service (IMPS)), and/or Short Message Service (SMS), or any other suitable communication protocol, including communication protocols not yet developed as of the filing date of this document.

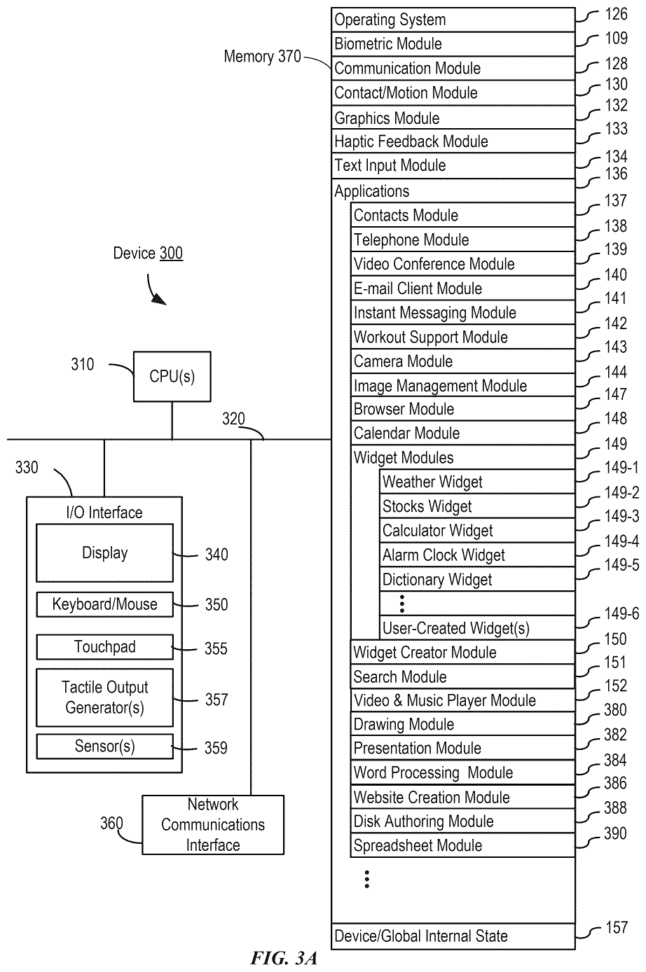

Audio circuitry 110, speaker 111, and microphone 113 provide an audio interface between a user and device 100. Audio circuitry 110 receives audio data from peripherals interface 118, converts the audio data to an electrical signal, and transmits the electrical signal to speaker 111. Speaker 111 converts the electrical signal to human-audible sound waves. Audio circuitry 110 also receives electrical signals converted by microphone 113 from sound waves. Audio circuitry 110 converts the electrical signal to audio data and transmits the audio data to peripherals interface 118 for processing. Audio data is, optionally, retrieved from and/or transmitted to memory 102 and/or RF circuitry 108 by peripherals interface 118. In some embodiments, audio circuitry 110 also includes a headset jack (e.g., 212, FIG. 2). The headset jack provides an interface between audio circuitry 110 and removable audio input/output peripherals, such as output-only headphones or a headset with both output (e.g., a headphone for one or both ears) and input (e.g., a microphone).

I/O subsystem 106 couples input/output peripherals on device 100, such as touch screen 112 and other input control devices 116, to peripherals interface 118. I/O subsystem 106 optionally includes display controller 156, optical sensor controller 158, depth camera controller 169, intensity sensor controller 159, haptic feedback controller 161, and one or more input controllers 160 for other input or control devices. The one or more input controllers 160 receive/send electrical signals from/to other input control devices 116. The other input control devices 116 optionally include physical buttons (e.g., push buttons, rocker buttons, etc.), dials, slider switches, joysticks, click wheels, and so forth. In some embodiments, input controller(s) 160 are, optionally, coupled to any (or none) of the following: a keyboard, an infrared port, a USB port, and a pointer device such as a mouse. The one or more buttons (e.g., 208, FIG. 2) optionally include an up/down button for volume control of speaker 111 and/or microphone 113. The one or more buttons optionally include a push button (e.g., 206, FIG. 2). In some embodiments, the electronic device is a computer system that is in communication (e.g., via wireless communication, via wired communication) with one or more input devices. In some embodiments, the one or more input devices include a touch-sensitive surface (e.g., a trackpad, as part of a touch-sensitive display). In some embodiments, the one or more input devices include one or more camera sensors (e.g., one or more optical sensors 164 and/or one or more depth camera sensors 175), such as for tracking a user's gestures (e.g., hand gestures and/or air gestures) as input. In some embodiments, the one or more input devices are integrated with the computer system. In some embodiments, the one or more input devices are separate from the computer system. In some embodiments, an air gesture is a gesture that is detected without the user touching an input element that is part of the device (or independently of an input element that is a part of the device) and is based on detected motion of a portion of the user's body through the air including motion of the user's body relative to an absolute reference (e.g., an angle of the user's arm relative to the ground or a distance of the user's hand relative to the ground), relative to another portion of the user's body (e.g., movement of a hand of the user relative to a shoulder of the user, movement of one hand of the user relative to another hand of the user, and/or movement of a finger of the user relative to another finger or portion of a hand of the user), and/or absolute motion of a portion of the user's body (e.g., a tap gesture that includes movement of a hand in a predetermined pose by a predetermined amount and/or speed, or a shake gesture that includes a predetermined speed or amount of rotation of a portion of the user's body).

A quick press of the push button optionally disengages a lock of touch screen 112 or optionally begins a process that uses gestures on the touch screen to unlock the device, as described in U.S. patent application Ser. No. 11/322,549, “Unlocking a Device by Performing Gestures on an Unlock Image,” filed Dec. 23, 2005, U.S. Pat. No. 7,657,849, which is hereby incorporated by reference in its entirety. A longer press of the push button (e.g., 206) optionally turns power to device 100 on or off. The functionality of one or more of the buttons are, optionally, user-customizable. Touch screen 112 is used to implement virtual or soft buttons and one or more soft keyboards.

Touch-sensitive display 112 provides an input interface and an output interface between the device and a user. Display controller 156 receives and/or sends electrical signals from/to touch screen 112. Touch screen 112 displays visual output to the user. The visual output optionally includes graphics, text, icons, video, and any combination thereof (collectively termed “graphics”). In some embodiments, some or all of the visual output optionally corresponds to user-interface objects.

Touch screen 112 has a touch-sensitive surface, sensor, or set of sensors that accepts input from the user based on haptic and/or tactile contact. Touch screen 112 and display controller 156 (along with any associated modules and/or sets of instructions in memory 102) detect contact (and any movement or breaking of the contact) on touch screen 112 and convert the detected contact into interaction with user-interface objects (e.g., one or more soft keys, icons, web pages, or images) that are displayed on touch screen 112. In an exemplary embodiment, a point of contact between touch screen 112 and the user corresponds to a finger of the user.

Touch screen 112 optionally uses LCD (liquid crystal display) technology, LPD (light emitting polymer display) technology, or LED (light emitting diode) technology, although other display technologies are used in other embodiments. Touch screen 112 and display controller 156 optionally detect contact and any movement or breaking thereof using any of a plurality of touch sensing technologies now known or later developed, including but not limited to capacitive, resistive, infrared, and surface acoustic wave technologies, as well as other proximity sensor arrays or other elements for determining one or more points of contact with touch screen 112. In an exemplary embodiment, projected mutual capacitance sensing technology is used, such as that found in the iPhone® and iPod Touch® from Apple Inc. of Cupertino, California.

A touch-sensitive display in some embodiments of touch screen 112 is, optionally, analogous to the multi-touch sensitive touchpads described in the following U.S. Pat. No. 6,323,846 (Westerman et al.), U.S. Pat. No. 6,570,557 (Westerman et al.), and/or U.S. Pat. No. 6,677,932 (Westerman), and/or U.S. Patent Publication 2002/0015024A1, each of which is hereby incorporated by reference in its entirety. However, touch screen 112 displays visual output from device 100, whereas touch-sensitive touchpads do not provide visual output.

A touch-sensitive display in some embodiments of touch screen 112 is described in the following applications: (1) U.S. patent application Ser. No. 11/381,313, “Multipoint Touch Surface Controller,” filed May 2, 2006; (2) U.S. patent application Ser. No. 10/840,862, “Multipoint Touchscreen,” filed May 6, 2004; (3) U.S. patent application Ser. No. 10/903,964, “Gestures For Touch Sensitive Input Devices,” filed Jul. 30, 2004; (4) U.S. patent application Ser. No. 11/048,264, “Gestures For Touch Sensitive Input Devices,” filed Jan. 31, 2005; (5) U.S. patent application Ser. No. 11/038,590, “Mode-Based Graphical User Interfaces For Touch Sensitive Input Devices,” filed Jan. 18, 2005; (6) U.S. patent application Ser. No. 11/228,758, “Virtual Input Device Placement On A Touch Screen User Interface,” filed Sep. 16, 2005; (7) U.S. patent application Ser. No. 11/228,700, “Operation Of A Computer With A Touch Screen Interface,” filed Sep. 16, 2005; (8) U.S. patent application Ser. No. 11/228,737, “Activating Virtual Keys Of A Touch-Screen Virtual Keyboard,” filed Sep. 16, 2005; and (9) U.S. patent application Ser. No. 11/367,749, “Multi-Functional Hand-Held Device,” filed Mar. 3, 2006. All of these applications are incorporated by reference herein in their entirety.

Touch screen 112 optionally has a video resolution in excess of 100 dpi. In some embodiments, the touch screen has a video resolution of approximately 160 dpi. The user optionally makes contact with touch screen 112 using any suitable object or appendage, such as a stylus, a finger, and so forth. In some embodiments, the user interface is designed to work primarily with finger-based contacts and gestures, which can be less precise than stylus-based input due to the larger area of contact of a finger on the touch screen. In some embodiments, the device translates the rough finger-based input into a precise pointer/cursor position or command for performing the actions desired by the user.

In some embodiments, in addition to the touch screen, device 100 optionally includes a touchpad for activating or deactivating particular functions. In some embodiments, the touchpad is a touch-sensitive area of the device that, unlike the touch screen, does not display visual output. The touchpad is, optionally, a touch-sensitive surface that is separate from touch screen 112 or an extension of the touch-sensitive surface formed by the touch screen.

Device 100 also includes power system 162 for powering the various components. Power system 162 optionally includes a power management system, one or more power sources (e.g., battery, alternating current (AC)), a recharging system, a power failure detection circuit, a power converter or inverter, a power status indicator (e.g., a light-emitting diode (LED)) and any other components associated with the generation, management and distribution of power in portable devices.

Device 100 optionally also includes secure element 163 for securely storing information. In some embodiments, secure element 163 is a hardware component (e.g., a secure microcontroller chip) configured to securely store data or an algorithm. In some embodiments, secure element 163 provides (e.g., releases) secure information (e.g., payment information (e.g., an account number and/or a transaction-specific dynamic security code), identification information (e.g., credentials of a state-approved digital identification), and/or authentication information (e.g., data generated using a cryptography engine and/or by performing asymmetric cryptography operations)). In some embodiments, secure element 163 provides (or releases) the secure information in response to device 100 receiving authorization, such as a user authentication (e.g., fingerprint authentication; passcode authentication; detecting double-press of a hardware button when device 100 is in an unlocked state, and optionally, while device 100 has been continuously on a user's wrist since device 100 was unlocked by providing authentication credentials to device 100, where the continuous presence of device 100 on the user's wrist is determined by periodically checking that the device is in contact with the user's skin). For example, device 100 detects a fingerprint at a fingerprint sensor (e.g., a fingerprint sensor integrated into a button) of device 100. Device 100 determines whether the detected fingerprint is consistent with an enrolled fingerprint. In accordance with a determination that the fingerprint is consistent with the enrolled fingerprint, secure element 163 provides (e.g., releases) the secure information. In accordance with a determination that the fingerprint is not consistent with the enrolled fingerprint, secure element 163 forgoes providing (e.g., releasing) the secure information.

Device 100 optionally also includes one or more optical sensors 164. FIG. 1A shows an optical sensor coupled to optical sensor controller 158 in I/O subsystem 106. Optical sensor 164 optionally includes charge-coupled device (CCD) or complementary metal-oxide semiconductor (CMOS) phototransistors. Optical sensor 164 receives light from the environment, projected through one or more lenses, and converts the light to data representing an image. In conjunction with imaging module 143 (also called a camera module), optical sensor 164 optionally captures still images or video. In some embodiments, an optical sensor is located on the back of device 100, opposite touch screen display 112 on the front of the device so that the touch screen display is enabled for use as a viewfinder for still and/or video image acquisition. In some embodiments, an optical sensor is located on the front of the device so that the user's image is, optionally, obtained for video conferencing while the user views the other video conference participants on the touch screen display. In some embodiments, the position of optical sensor 164 can be changed by the user (e.g., by rotating the lens and the sensor in the device housing) so that a single optical sensor 164 is used along with the touch screen display for both video conferencing and still and/or video image acquisition.

Device 100 optionally also includes one or more depth camera sensors 175. FIG. 1A shows a depth camera sensor coupled to depth camera controller 169 in I/O subsystem 106. Depth camera sensor 175 receives data from the environment to create a three dimensional model of an object (e.g., a face) within a scene from a viewpoint (e.g., a depth camera sensor). In some embodiments, in conjunction with imaging module 143 (also called a camera module), depth camera sensor 175 is optionally used to determine a depth map of different portions of an image captured by the imaging module 143. In some embodiments, a depth camera sensor is located on the front of device 100 so that the user's image with depth information is, optionally, obtained for video conferencing while the user views the other video conference participants on the touch screen display and to capture selfies with depth map data. In some embodiments, the depth camera sensor 175 is located on the back of device, or on the back and the front of the device 100. In some embodiments, the position of depth camera sensor 175 can be changed by the user (e.g., by rotating the lens and the sensor in the device housing) so that a depth camera sensor 175 is used along with the touch screen display for both video conferencing and still and/or video image acquisition.

Device 100 optionally also includes one or more contact intensity sensors 165. FIG. 1A shows a contact intensity sensor coupled to intensity sensor controller 159 in I/O subsystem 106. Contact intensity sensor 165 optionally includes one or more piezoresistive strain gauges, capacitive force sensors, electric force sensors, piezoelectric force sensors, optical force sensors, capacitive touch-sensitive surfaces, or other intensity sensors (e.g., sensors used to measure the force (or pressure) of a contact on a touch-sensitive surface). Contact intensity sensor 165 receives contact intensity information (e.g., pressure information or a proxy for pressure information) from the environment. In some embodiments, at least one contact intensity sensor is collocated with, or proximate to, a touch-sensitive surface (e.g., touch-sensitive display system 112). In some embodiments, at least one contact intensity sensor is located on the back of device 100, opposite touch screen display 112, which is located on the front of device 100.

Device 100 optionally also includes one or more proximity sensors 166. FIG. 1A shows proximity sensor 166 coupled to peripherals interface 118. Alternately, proximity sensor 166 is, optionally, coupled to input controller 160 in I/O subsystem 106. Proximity sensor 166 optionally performs as described in U.S. patent application Ser. No. 11/241,839, “Proximity Detector In Handheld Device”; Ser. No. 11/240,788, “Proximity Detector In Handheld Device”; Ser. No. 11/620,702, “Using Ambient Light Sensor To Augment Proximity Sensor Output”; Ser. No. 11/586,862, “Automated Response To And Sensing Of User Activity In Portable Devices”; and Ser. No. 11/638,251, “Methods And Systems For Automatic Configuration Of Peripherals,” which are hereby incorporated by reference in their entirety. In some embodiments, the proximity sensor turns off and disables touch screen 112 when the multifunction device is placed near the user's ear (e.g., when the user is making a phone call).

Device 100 optionally also includes one or more tactile output generators 167. FIG. 1A shows a tactile output generator coupled to haptic feedback controller 161 in I/O subsystem 106. Tactile output generator 167 optionally includes one or more electroacoustic devices such as speakers or other audio components and/or electromechanical devices that convert energy into linear motion such as a motor, solenoid, electroactive polymer, piezoelectric actuator, electrostatic actuator, or other tactile output generating component (e.g., a component that converts electrical signals into tactile outputs on the device). Contact intensity sensor 165 receives tactile feedback generation instructions from haptic feedback module 133 and generates tactile outputs on device 100 that are capable of being sensed by a user of device 100. In some embodiments, at least one tactile output generator is collocated with, or proximate to, a touch-sensitive surface (e.g., touch-sensitive display system 112) and, optionally, generates a tactile output by moving the touch-sensitive surface vertically (e.g., in/out of a surface of device 100) or laterally (e.g., back and forth in the same plane as a surface of device 100). In some embodiments, at least one tactile output generator sensor is located on the back of device 100, opposite touch screen display 112, which is located on the front of device 100.

Device 100 optionally also includes one or more accelerometers 168. FIG. 1A shows accelerometer 168 coupled to peripherals interface 118. Alternately, accelerometer 168 is, optionally, coupled to an input controller 160 in I/O subsystem 106. Accelerometer 168 optionally performs as described in U.S. Patent Publication No. 20050190059, “Acceleration-based Theft Detection System for Portable Electronic Devices,” and U.S. Patent Publication No. 20060017692, “Methods And Apparatuses For Operating A Portable Device Based On An Accelerometer,” both of which are incorporated by reference herein in their entirety. In some embodiments, information is displayed on the touch screen display in a portrait view or a landscape view based on an analysis of data received from the one or more accelerometers. Device 100 optionally includes, in addition to accelerometer(s) 168, a magnetometer and a GPS (or GLONASS or other global navigation system) receiver for obtaining information concerning the location and orientation (e.g., portrait or landscape) of device 100.

In some embodiments, the software components stored in memory 102 include operating system 126, biometric module 109, communication module (or set of instructions) 128, contact/motion module (or set of instructions) 130, graphics module (or set of instructions) 132, text input module (or set of instructions) 134, Global Positioning System (GPS) module (or set of instructions) 135, authentication module 105, and applications (or sets of instructions) 136.

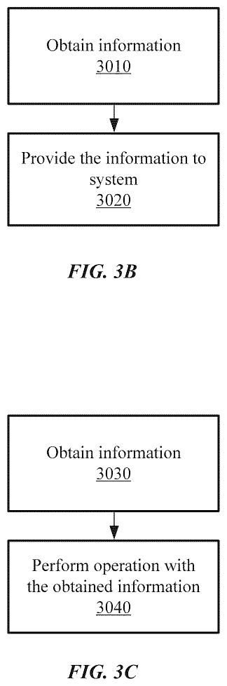

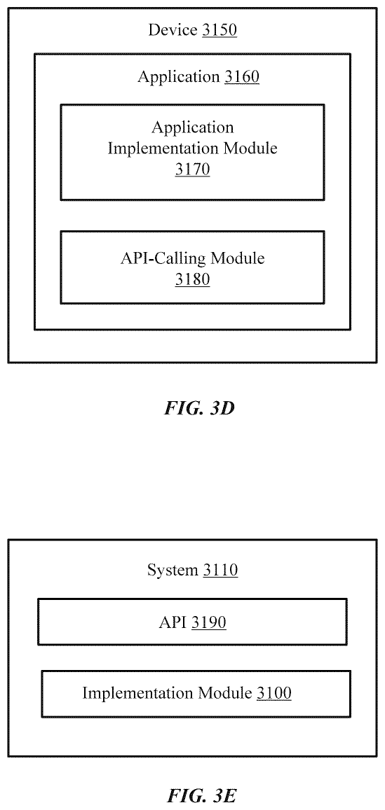

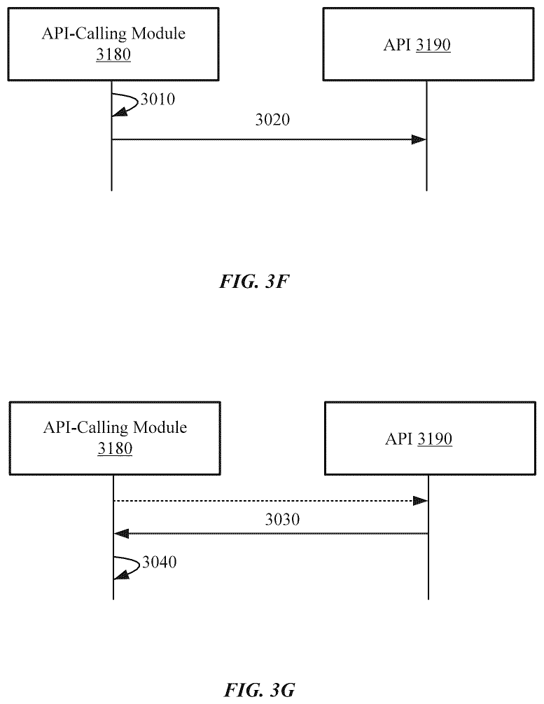

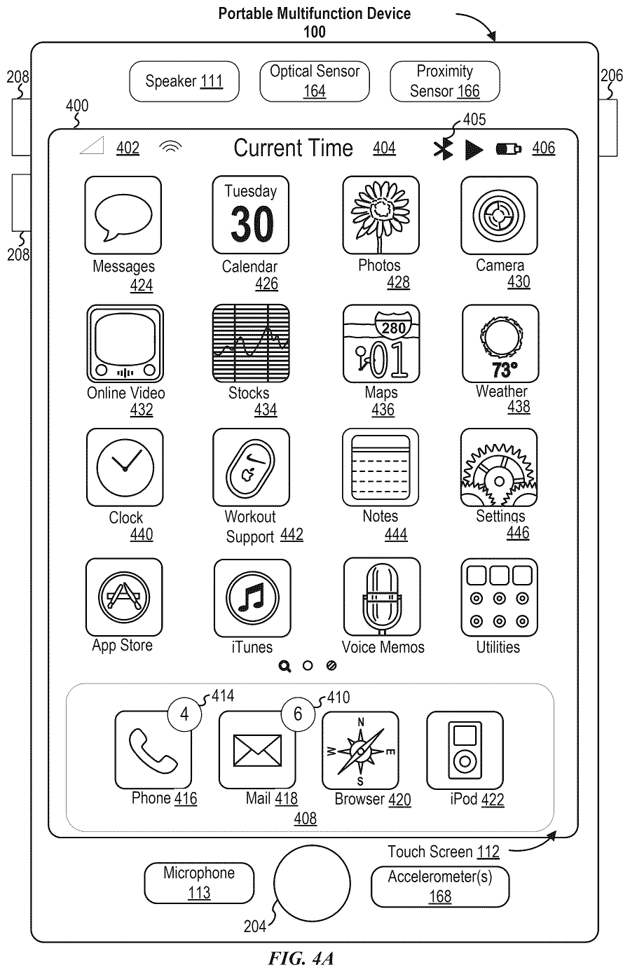

Furthermore, in some embodiments, memory 102 (FIG. 1A) or 370 (FIG. 3A) stores device/global internal state 157, as shown in FIGS. 1A and 3A. Device/global internal state 157 includes one or more of: active application state, indicating which applications, if any, are currently active; display state, indicating what applications, views or other information occupy various regions of touch screen display 112; sensor state, including information obtained from the device's various sensors and input control devices 116; and location information concerning the device's location and/or attitude.

Operating system 126 (e.g., Darwin, RTXC, LINUX, UNIX, OS X, IOS, WINDOWS, or an embedded operating system such as VxWorks) includes various software components and/or drivers for controlling and managing general system tasks (e.g., memory management, storage device control, power management, etc.) and facilitates communication between various hardware and software components.

Communication module 128 facilitates communication with other devices over one or more external ports 124 and also includes various software components for handling data received by RF circuitry 108 and/or external port 124. External port 124 (e.g., Universal Serial Bus (USB), FIREWIRE®, etc.) is adapted for coupling directly to other devices or indirectly over a network (e.g., the Internet, wireless LAN, etc.). In some embodiments, the external port is a multi-pin (e.g., 30-pin) connector that is the same as, or similar to and/or compatible with, the 30-pin connector used on iPod® (trademark of Apple Inc.) devices.

Biometric module 109 optionally stores information about one or more enrolled biometric features (e.g., fingerprint feature information, facial recognition feature information, eye and/or iris feature information) for use to verify whether received biometric information matches the enrolled biometric features. In some embodiments, the information stored about the one or more enrolled biometric features includes data that enables the comparison between the stored information and received biometric information without including enough information to reproduce the enrolled biometric features. In some embodiments, biometric module 109 stores the information about the enrolled biometric features in association with a user account of device 100. In some embodiments, biometric module 109 compares the received biometric information to an enrolled biometric feature to determine whether the received biometric information matches the enrolled biometric feature.

Contact/motion module 130 optionally detects contact with touch screen 112 (in conjunction with display controller 156) and other touch-sensitive devices (e.g., a touchpad or physical click wheel). Contact/motion module 130 includes various software components for performing various operations related to detection of contact, such as determining if contact has occurred (e.g., detecting a finger-down event), determining an intensity of the contact (e.g., the force or pressure of the contact or a substitute for the force or pressure of the contact), determining if there is movement of the contact and tracking the movement across the touch-sensitive surface (e.g., detecting one or more finger-dragging events), and determining if the contact has ceased (e.g., detecting a finger-up event or a break in contact). Contact/motion module 130 receives contact data from the touch-sensitive surface. Determining movement of the point of contact, which is represented by a series of contact data, optionally includes determining speed (magnitude), velocity (magnitude and direction), and/or an acceleration (a change in magnitude and/or direction) of the point of contact. These operations are, optionally, applied to single contacts (e.g., one finger contacts) or to multiple simultaneous contacts (e.g., “multitouch”/multiple finger contacts). In some embodiments, contact/motion module 130 and display controller 156 detect contact on a touchpad.

In some embodiments, contact/motion module 130 uses a set of one or more intensity thresholds to determine whether an operation has been performed by a user (e.g., to determine whether a user has “clicked” on an icon). In some embodiments, at least a subset of the intensity thresholds are determined in accordance with software parameters (e.g., the intensity thresholds are not determined by the activation thresholds of particular physical actuators and can be adjusted without changing the physical hardware of device 100). For example, a mouse “click” threshold of a trackpad or touch screen display can be set to any of a large range of predefined threshold values without changing the trackpad or touch screen display hardware. Additionally, in some implementations, a user of the device is provided with software settings for adjusting one or more of the set of intensity thresholds (e.g., by adjusting individual intensity thresholds and/or by adjusting a plurality of intensity thresholds at once with a system-level click “intensity” parameter).

Contact/motion module 130 optionally detects a gesture input by a user. Different gestures on the touch-sensitive surface have different contact patterns (e.g., different motions, timings, and/or intensities of detected contacts). Thus, a gesture is, optionally, detected by detecting a particular contact pattern. For example, detecting a finger tap gesture includes detecting a finger-down event followed by detecting a finger-up (liftoff) event at the same position (or substantially the same position) as the finger-down event (e.g., at the position of an icon). As another example, detecting a finger swipe gesture on the touch-sensitive surface includes detecting a finger-down event followed by detecting one or more finger-dragging events, and subsequently followed by detecting a finger-up (liftoff) event.

Graphics module 132 includes various known software components for rendering and displaying graphics on touch screen 112 or other display, including components for changing the visual impact (e.g., brightness, transparency, saturation, contrast, or other visual property) of graphics that are displayed. As used herein, the term “graphics” includes any object that can be displayed to a user, including, without limitation, text, web pages, icons (such as user-interface objects including soft keys), digital images, videos, animations, and the like.

In some embodiments, graphics module 132 stores data representing graphics to be used. Each graphic is, optionally, assigned a corresponding code. Graphics module 132 receives, from applications etc., one or more codes specifying graphics to be displayed along with, if necessary, coordinate data and other graphic property data, and then generates screen image data to output to display controller 156.

Haptic feedback module 133 includes various software components for generating instructions used by tactile output generator(s) 167 to produce tactile outputs at one or more locations on device 100 in response to user interactions with device 100.

Text input module 134, which is, optionally, a component of graphics module 132, provides soft keyboards for entering text in various applications (e.g., contacts module 137, e-mail client module 140, IM module 141, browser module 147, and any other application that needs text input).

GPS module 135 determines the location of the device and provides this information for use in various applications (e.g., to telephone module 138 for use in location-based dialing; to camera module 143 as picture/video metadata; and to applications that provide location-based services such as weather widgets, local yellow page widgets, and map/navigation widgets).

Authentication module 105 determines whether a requested operation (e.g., requested by an application of applications 136) is authorized to be performed. In some embodiments, authentication module 105 receives for an operation to be perform that optionally requires authentication. Authentication module 105 determines whether the operation is authorized to be performed, such as based on a series of factors, including the lock status of device 100, the location of device 100, whether a security delay has elapsed, whether received biometric information matches enrolled biometric features, and/or other factors. Once authentication module 105 determines that the operation is authorized to be performed, authentication module 105 triggers performance of the operation.

Applications 136 optionally include the following modules (or sets of instructions), or a subset or superset thereof:
Contacts module 137 (sometimes called an address book or contact list);
Telephone module 138;
Video conference module 139;
E-mail client module 140;
Instant messaging (IM) module 141;
Workout support module 142;
Camera module 143 for still and/or video images;
Image management module 144;

Video player module;
Music player module;
Browser module 147;
Calendar module 148;
Widget modules 149, which optionally include one or more of: weather widget 149-1, stocks widget 149-2, calculator widget 149-3, alarm clock widget 149-4, dictionary widget 149-5, and other widgets obtained by the user, as well as user-created widgets 149-6;
Widget creator module 150 for making user-created widgets 149-6;
Search module 151;
Video and music player module 152, which merges video player module and music player module;
Notes module 153;
Map module 154; and/or
Online video module 155.

Examples of other applications 136 that are, optionally, stored in memory 102 include other word processing applications, other image editing applications, drawing applications, presentation applications, JAVA-enabled applications, encryption, digital rights management, voice recognition, and voice replication.

In conjunction with touch screen 112, display controller 156, contact/motion module 130, graphics module 132, and text input module 134, contacts module 137 are, optionally, used to manage an address book or contact list (e.g., stored in application internal state 192 of contacts module 137 in memory 102 or memory 370), including: adding name(s) to the address book; deleting name(s) from the address book; associating telephone number(s), e-mail address(es), physical address(es) or other information with a name; associating an image with a name; categorizing and sorting names; providing telephone numbers or e-mail addresses to initiate and/or facilitate communications by telephone module 138, video conference module 139, e-mail client module 140, or IM module 141; and so forth.

In conjunction with RF circuitry 108, audio circuitry 110, speaker 111, microphone 113, touch screen 112, display controller 156, contact/motion module 130, graphics module 132, and text input module 134, telephone module 138 are optionally, used to enter a sequence of characters corresponding to a telephone number, access one or more telephone numbers in contacts module 137, modify a telephone number that has been entered, dial a respective telephone number, conduct a conversation, and disconnect or hang up when the conversation is completed. As noted above, the wireless communication optionally uses any of a plurality of communications standards, protocols, and technologies.

In conjunction with RF circuitry 108, audio circuitry 110, speaker 111, microphone 113, touch screen 112, display controller 156, optical sensor 164, optical sensor controller 158, contact/motion module 130, graphics module 132, text input module 134, contacts module 137, and telephone module 138, video conference module 139 includes executable instructions to initiate, conduct, and terminate a video conference between a user and one or more other participants in accordance with user instructions.

In conjunction with RF circuitry 108, touch screen 112, display controller 156, contact/motion module 130, graphics module 132, and text input module 134, e-mail client module 140 includes executable instructions to create, send, receive, and manage e-mail in response to user instructions. In conjunction with image management module 144, e-mail client module 140 makes it very easy to create and send e-mails with still or video images taken with camera module 143.

In conjunction with RF circuitry 108, touch screen 112, display controller 156, contact/motion module 130, graphics module 132, and text input module 134, the instant messaging module 141 includes executable instructions to enter a sequence of characters corresponding to an instant message, to modify previously entered characters, to transmit a respective instant message (for example, using a Short Message Service (SMS) or Multimedia Message Service (MMS) protocol for telephony-based instant messages or using XMPP, SIMPLE, or IMPS for Internet-based instant messages), to receive instant messages, and to view received instant messages. In some embodiments, transmitted and/or received instant messages optionally include graphics, photos, audio files, video files and/or other attachments as are supported in an MMS and/or an Enhanced Messaging Service (EMS). As used herein, “instant messaging” refers to both telephony-based messages (e.g., messages sent using SMS or MMS) and Internet-based messages (e.g., messages sent using XMPP, SIMPLE, or IMPS).

In conjunction with RF circuitry 108, touch screen 112, display controller 156, contact/motion module 130, graphics module 132, text input module 134, GPS module 135, map module 154, and music player module, workout support module 142 includes executable instructions to create workouts (e.g., with time, distance, and/or calorie burning goals); communicate with workout sensors (sports devices); receive workout sensor data; calibrate sensors used to monitor a workout; select and play music for a workout; and display, store, and transmit workout data.

In conjunction with touch screen 112, display controller 156, optical sensor(s) 164, optical sensor controller 158, contact/motion module 130, graphics module 132, and image management module 144, camera module 143 includes executable instructions to capture still images or video (including a video stream) and store them into memory 102, modify characteristics of a still image or video, or delete a still image or video from memory 102.

In conjunction with touch screen 112, display controller 156, contact/motion module 130, graphics module 132, text input module 134, and camera module 143, image management module 144 includes executable instructions to arrange, modify (e.g., edit), or otherwise manipulate, label, delete, present (e.g., in a digital slide show or album), and store still and/or video images.

In conjunction with RF circuitry 108, touch screen 112, display controller 156, contact/motion module 130, graphics module 132, and text input module 134, browser module 147 includes executable instructions to browse the Internet in accordance with user instructions, including searching, linking to, receiving, and displaying web pages or portions thereof, as well as attachments and other files linked to web pages.

In conjunction with RF circuitry 108, touch screen 112, display controller 156, contact/motion module 130, graphics module 132, text input module 134, e-mail client module 140, and browser module 147, calendar module 148 includes executable instructions to create, display, modify, and store calendars and data associated with calendars (e.g., calendar entries, to-do lists, etc.) in accordance with user instructions.

In conjunction with RF circuitry 108, touch screen 112, display controller 156, contact/motion module 130, graphics module 132, text input module 134, and browser module 147, widget modules 149 are mini-applications that are, optionally, downloaded and used by a user (e.g., weather widget 149-1, stocks widget 149-2, calculator widget 149-3, alarm clock widget 149-4, and dictionary widget 149-5) or created by the user (e.g., user-created widget 149-6). In some embodiments, a widget includes an HTML (Hypertext Markup Language) file, a CSS (Cascading Style Sheets) file, and a JavaScript® file. In some embodiments, a widget includes an XML (Extensible Markup Language) file and a JavaScript® file (e.g., Yahoo!® Widgets).

In conjunction with RF circuitry 108, touch screen 112, display controller 156, contact/motion module 130, graphics module 132, text input module 134, and browser module 147, the widget creator module 150 are, optionally, used by a user to create widgets (e.g., turning a user-specified portion of a web page into a widget).

In conjunction with touch screen 112, display controller 156, contact/motion module 130, graphics module 132, and text input module 134, search module 151 includes executable instructions to search for text, music, sound, image, video, and/or other files in memory 102 that match one or more search criteria (e.g., one or more user-specified search terms) in accordance with user instructions.

In conjunction with touch screen 112, display controller 156, contact/motion module 130, graphics module 132, audio circuitry 110, speaker 111, RF circuitry 108, and browser module 147, video and music player module 152 includes executable instructions that allow the user to download and play back recorded music and other sound files stored in one or more file formats, such as MP3 or AAC files, and executable instructions to display, present, or otherwise play back videos (e.g., on touch screen 112 or on an external, connected display via external port 124). In some embodiments, device 100 optionally includes the functionality of an MP3 player, such as an iPod (trademark of Apple Inc.).

In conjunction with touch screen 112, display controller 156, contact/motion module 130, graphics module 132, and text input module 134, notes module 153 includes executable instructions to create and manage notes, to-do lists, and the like in accordance with user instructions.

In conjunction with RF circuitry 108, touch screen 112, display controller 156, contact/motion module 130, graphics module 132, text input module 134, GPS module 135, and browser module 147, map module 154 are, optionally, used to receive, display, modify, and store maps and data associated with maps (e.g., driving directions, data on stores and other points of interest at or near a particular location, and other location-based data) in accordance with user instructions.

In conjunction with touch screen 112, display controller 156, contact/motion module 130, graphics module 132, audio circuitry 110, speaker 111, RF circuitry 108, text input module 134, e-mail client module 140, and browser module 147, online video module 155 includes instructions that allow the user to access, browse, receive (e.g., by streaming and/or download), play back (e.g., on the touch screen or on an external, connected display via external port 124), send an e-mail with a link to a particular online video, and otherwise manage online videos in one or more file formats, such as H.264. In some embodiments, instant messaging module 141, rather than e-mail client module 140, is used to send a link to a particular online video. Additional description of the online video application can be found in U.S. Provisional Patemt Application No. 60/936,562, “Portable Multifunction Device, Method, and Graphical User Interface for Playing Online Videos,” filed Jun. 20, 2007, and U.S. patent application Ser. No. 11/968,067, “Portable Multifunction Device, Method, and Graphical User Interface for Playing Online Videos,” filed Dec. 31, 2007, the contents of which are hereby incorporated by reference in their entirety.

Each of the above-identified modules and applications corresponds to a set of executable instructions for performing one or more functions described above and the methods described in this application (e.g., the computer-implemented methods and other information processing methods described herein). These modules (e.g., sets of instructions) need not be implemented as separate software programs (such as computer programs (e.g., including instructions)), procedures, or modules, and thus various subsets of these modules are, optionally, combined or otherwise rearranged in various embodiments. For example, video player module is, optionally, combined with music player module into a single module (e.g., video and music player module 152, FIG. 1A). In some embodiments, memory 102 optionally stores a subset of the modules and data structures identified above. Furthermore, memory 102 optionally stores additional modules and data structures not described above.

In some embodiments, device 100 is a device where operation of a predefined set of functions on the device is performed exclusively through a touch screen and/or a touchpad. By using a touch screen and/or a touchpad as the primary input control device for operation of device 100, the number of physical input control devices (such as push buttons, dials, and the like) on device 100 is, optionally, reduced.

The predefined set of functions that are performed exclusively through a touch screen and/or a touchpad optionally include navigation between user interfaces. In some embodiments, the touchpad, when touched by the user, navigates device 100 to a main, home, or root menu from any user interface that is displayed on device 100. In such embodiments, a “menu button” is implemented using a touchpad. In some other embodiments, the menu button is a physical push button or other physical input control device instead of a touchpad.

FIG. 1B is a block diagram illustrating exemplary components for event handling in accordance with some embodiments. In some embodiments, memory 102 (FIG. 1A) or 370 (FIG. 3A) includes event sorter 170 (e.g., in operating system 126) and a respective application 136-1 (e.g., any of the aforementioned applications 137-151, 155, 380-390).

Event sorter 170 receives event information and determines the application 136-1 and application view 191 of application 136-1 to which to deliver the event information. Event sorter 170 includes event monitor 171 and event dispatcher module 174. In some embodiments, application 136-1 includes application internal state 192, which indicates the current application view(s) displayed on touch-sensitive display 112 when the application is active or executing. In some embodiments, device/global internal state 157 is used by event sorter 170 to determine which application(s) is (are) currently active, and application internal state 192 is used by event sorter 170 to determine application views 191 to which to deliver event information.

In some embodiments, application internal state 192 includes additional information, such as one or more of: resume information to be used when application 136-1 resumes execution, user interface state information that indicates information being displayed or that is ready for display by application 136-1, a state queue for enabling the user to go back to a prior state or view of application 136-1, and a redo/undo queue of previous actions taken by the user.

Event monitor 171 receives event information from peripherals interface 118. Event information includes information about a sub-event (e.g., a user touch on touch-sensitive display 112, as part of a multi-touch gesture). Peripherals interface 118 transmits information it receives from I/O subsystem 106 or a sensor, such as proximity sensor 166, accelerometer(s) 168, and/or microphone 113 (through audio circuitry 110). Information that peripherals interface 118 receives from I/O subsystem 106 includes information from touch-sensitive display 112 or a touch-sensitive surface.

In some embodiments, event monitor 171 sends requests to the peripherals interface 118 at predetermined intervals. In response, peripherals interface 118 transmits event information. In other embodiments, peripherals interface 118 transmits event information only when there is a significant event (e.g., receiving an input above a predetermined noise threshold and/or for more than a predetermined duration).

In some embodiments, event sorter 170 also includes a hit view determination module 172 and/or an active event recognizer determination module 173.

Hit view determination module 172 provides software procedures for determining where a sub-event has taken place within one or more views when touch-sensitive display 112 displays more than one view. Views are made up of controls and other elements that a user can see on the display.

Another aspect of the user interface associated with an application is a set of views, sometimes herein called application views or user interface windows, in which information is displayed and touch-based gestures occur. The application views (of a respective application) in which a touch is detected optionally correspond to programmatic levels within a programmatic or view hierarchy of the application. For example, the lowest level view in which a touch is detected is, optionally, called the hit view, and the set of events that are recognized as proper inputs are, optionally, determined based, at least in part, on the hit view of the initial touch that begins a touch-based gesture.

Hit view determination module 172 receives information related to sub-events of a touch-based gesture. When an application has multiple views organized in a hierarchy, hit view determination module 172 identifies a hit view as the lowest view in the hierarchy which should handle the sub-event. In most circumstances, the hit view is the lowest level view in which an initiating sub-event occurs (e.g., the first sub-event in the sequence of sub-events that form an event or potential event). Once the hit view is identified by the hit view determination module 172, the hit view typically receives all sub-events related to the same touch or input source for which it was identified as the hit view.

Active event recognizer determination module 173 determines which view or views within a view hierarchy should receive a particular sequence of sub-events. In some embodiments, active event recognizer determination module 173 determines that only the hit view should receive a particular sequence of sub-events. In other embodiments, active event recognizer determination module 173 determines that all views that include the physical location of a sub-event are actively involved views, and therefore determines that all actively involved views should receive a particular sequence of sub-events. In other embodiments, even if touch sub-events were entirely confined to the area associated with one particular view, views higher in the hierarchy would still remain as actively involved views.

Event dispatcher module 174 dispatches the event information to an event recognizer (e.g., event recognizer 180). In embodiments including active event recognizer determination module 173, event dispatcher module 174 delivers the event information to an event recognizer determined by active event recognizer determination module 173. In some embodiments, event dispatcher module 174 stores in an event queue the event information, which is retrieved by a respective event receiver 182.

In some embodiments, operating system 126 includes event sorter 170. Alternatively, application 136-1 includes event sorter 170. In yet other embodiments, event sorter 170 is a stand-alone module, or a part of another module stored in memory 102, such as contact/motion module 130.

In some embodiments, application 136-1 includes a plurality of event handlers 190 and one or more application views 191, each of which includes instructions for handling touch events that occur within a respective view of the application's user interface. Each application view 191 of the application 136-1 includes one or more event recognizers 180. Typically, a respective application view 191 includes a plurality of event recognizers 180. In other embodiments, one or more of event recognizers 180 are part of a separate module, such as a user interface kit or a higher level object from which application 136-1 inherits methods and other properties. In some embodiments, a respective event handler 190 includes one or more of: data updater 176, object updater 177, GUI updater 178, and/or event data 179 received from event sorter 170. Event handler 190 optionally utilizes or calls data updater 176, object updater 177, or GUI updater 178 to update the application internal state 192. Alternatively, one or more of the application views 191 include one or more respective event handlers 190. Also, in some embodiments, one or more of data updater 176, object updater 177, and GUI updater 178 are included in a respective application view 191.

A respective event recognizer 180 receives event information (e.g., event data 179) from event sorter 170 and identifies an event from the event information. Event recognizer 180 includes event receiver 182 and event comparator 184. In some embodiments, event recognizer 180 also includes at least a subset of: metadata 183, and event delivery instructions 188 (which optionally include sub-event delivery instructions).

Event receiver 182 receives event information from event sorter 170. The event information includes information about a sub-event, for example, a touch or a touch movement. Depending on the sub-event, the event information also includes additional information, such as location of the sub-event. When the sub-event concerns motion of a touch, the event information optionally also includes speed and direction of the sub-event. In some embodiments, events include rotation of the device from one orientation to another (e.g., from a portrait orientation to a landscape orientation, or vice versa), and the event information includes corresponding information about the current orientation (also called device attitude) of the device.

Event comparator 184 compares the event information to predefined event or sub-event definitions and, based on the comparison, determines an event or sub-event, or determines or updates the state of an event or sub-event. In some embodiments, event comparator 184 includes event definitions 186. Event definitions 186 contain definitions of events (e.g., predefined sequences of sub-events), for example, event 1 (187-1), event 2 (187-2), and others. In some embodiments, sub-events in an event (e.g., 187-1 and/or 187-2) include, for example, touch begin, touch end, touch movement, touch cancellation, and multiple touching. In one example, the definition for event 1 (187-1) is a double tap on a displayed object. The double tap, for example, comprises a first touch (touch begin) on the displayed object for a predetermined phase, a first liftoff (touch end) for a predetermined phase, a second touch (touch begin) on the displayed object for a predetermined phase, and a second liftoff (touch end) for a predetermined phase. In another example, the definition for event 2 (187-2) is a dragging on a displayed object. The dragging, for example, comprises a touch (or contact) on the displayed object for a predetermined phase, a movement of the touch across touch-sensitive display 112, and liftoff of the touch (touch end). In some embodiments, the event also includes information for one or more associated event handlers 190.

In some embodiments, event definitions 186 include a definition of an event for a respective user-interface object. In some embodiments, event comparator 184 performs a hit test to determine which user-interface object is associated with a sub-event. For example, in an application view in which three user-interface objects are displayed on touch-sensitive display 112, when a touch is detected on touch-sensitive display 112, event comparator 184 performs a hit test to determine which of the three user-interface objects is associated with the touch (sub-event). If each displayed object is associated with a respective event handler 190, the event comparator uses the result of the hit test to determine which event handler 190 should be activated. For example, event comparator 184 selects an event handler associated with the sub-event and the object triggering the hit test.

In some embodiments, the definition for a respective event (187) also includes delayed actions that delay delivery of the event information until after it has been determined whether the sequence of sub-events does or does not correspond to the event recognizer's event type.

When a respective event recognizer 180 determines that the series of sub-events do not match any of the events in event definitions 186, the respective event recognizer 180 enters an event impossible, event failed, or event ended state, after which it disregards subsequent sub-events of the touch-based gesture. In this situation, other event recognizers, if any, that remain active for the hit view continue to track and process sub-events of an ongoing touch-based gesture.

In some embodiments, a respective event recognizer 180 includes metadata 183 with configurable properties, flags, and/or lists that indicate how the event delivery system should perform sub-event delivery to actively involved event recognizers. In some embodiments, metadata 183 includes configurable properties, flags, and/or lists that indicate how event recognizers interact, or are enabled to interact, with one another. In some embodiments, metadata 183 includes configurable properties, flags, and/or lists that indicate whether sub-events are delivered to varying levels in the view or programmatic hierarchy.

In some embodiments, a respective event recognizer 180 activates event handler 190 associated with an event when one or more particular sub-events of an event are recognized. In some embodiments, a respective event recognizer 180 delivers event information associated with the event to event handler 190. Activating an event handler 190 is distinct from sending (and deferred sending) sub-events to a respective hit view. In some embodiments, event recognizer 180 throws a flag associated with the recognized event, and event handler 190 associated with the flag catches the flag and performs a predefined process.

In some embodiments, event delivery instructions 188 include sub-event delivery instructions that deliver event information about a sub-event without activating an event handler. Instead, the sub-event delivery instructions deliver event information to event handlers associated with the series of sub-events or to actively involved views. Event handlers associated with the series of sub-events or with actively involved views receive the event information and perform a predetermined process.

In some embodiments, data updater 176 creates and updates data used in application 136-1. For example, data updater 176 updates the telephone number used in contacts module 137, or stores a video file used in video player module. In some embodiments, object updater 177 creates and updates objects used in application 136-1. For example, object updater 177 creates a new user-interface object or updates the position of a user-interface object. GUI updater 178 updates the GUI. For example, GUI updater 178 prepares display information and sends it to graphics module 132 for display on a touch-sensitive display.

In some embodiments, event handler(s) 190 includes or has access to data updater 176, object updater 177, and GUI updater 178. In some embodiments, data updater 176, object updater 177, and GUI updater 178 are included in a single module of a respective application 136-1 or application view 191. In other embodiments, they are included in two or more software modules.

It shall be understood that the foregoing discussion regarding event handling of user touches on touch-sensitive displays also applies to other forms of user inputs to operate multifunction devices 100 with input devices, not all of which are initiated on touch screens. For example, mouse movement and mouse button presses, optionally coordinated with single or multiple keyboard presses or holds; contact movements such as taps, drags, scrolls, etc. on touchpads; pen stylus inputs; movement of the device; oral instructions; detected eye movements; biometric inputs; and/or any combination thereof are optionally utilized as inputs corresponding to sub-events which define an event to be recognized.

FIG. 2 illustrates a portable multifunction device 100 having a touch screen 112 in accordance with some embodiments. The touch screen optionally displays one or more graphics within user interface (UI) 200. In this embodiment, as well as others described below, a user is enabled to select one or more of the graphics by making a gesture on the graphics, for example, with one or more fingers 202 (not drawn to scale in the figure) or one or more styluses 203 (not drawn to scale in the figure). In some embodiments, selection of one or more graphics occurs when the user breaks contact with the one or more graphics. In some embodiments, the gesture optionally includes one or more taps, one or more swipes (from left to right, right to left, upward and/or downward), and/or a rolling of a finger (from right to left, left to right, upward and/or downward) that has made contact with device 100. In some implementations or circumstances, inadvertent contact with a graphic does not select the graphic. For example, a swipe gesture that sweeps over an application icon optionally does not select the corresponding application when the gesture corresponding to selection is a tap.

Device 100 optionally also include one or more physical buttons, such as “home” or menu button 204. As described previously, menu button 204 is, optionally, used to navigate to any application 136 in a set of applications that are, optionally, executed on device 100. Alternatively, in some embodiments, the menu button is implemented as a soft key in a GUI displayed on touch screen 112.

In some embodiments, device 100 includes touch screen 112, menu button 204, push button 206 for powering the device on/off and locking the device, volume adjustment button(s) 208, subscriber identity module (SIM) card slot 210, headset jack 212, and docking/charging external port 124. Push button 206 is, optionally, used to turn the power on/off on the device by depressing the button and holding the button in the depressed state for a predefined time interval; to lock the device by depressing the button and releasing the button before the predefined time interval has elapsed; and/or to unlock the device or initiate an unlock process. In an alternative embodiment, device 100 also accepts verbal input for activation or deactivation of some functions through microphone 113. Device 100 also, optionally, includes one or more contact intensity sensors 165 for detecting intensity of contacts on touch screen 112 and/or one or more tactile output generators 167 for generating tactile outputs for a user of device 100.

FIG. 3A is a block diagram of an exemplary multifunction device with a display and a touch-sensitive surface in accordance with some embodiments. Device 300 need not be portable. In some embodiments, device 300 is a laptop computer, a desktop computer, a tablet computer, a multimedia player device, a navigation device, an educational device (such as a child's learning toy), a gaming system, or a control device (e.g., a home or industrial controller). Device 300 typically includes one or more processing units (CPUs) 310, one or more network or other communications interfaces 360, memory 370, and one or more communication buses 320 for interconnecting these components. Communication buses 320 optionally include circuitry (sometimes called a chipset) that interconnects and controls communications between system components. Device 300 includes input/output (I/O) interface 330 comprising display 340, which is typically a touch screen display. I/O interface 330 also optionally includes a keyboard and/or mouse (or other pointing device) 350 and touchpad 355, tactile output generator 357 for generating tactile outputs on device 300 (e.g., similar to tactile output generator(s) 167 described above with reference to FIG. 1A), sensors 359 (e.g., optical, acceleration, proximity, touch-sensitive, and/or contact intensity sensors similar to contact intensity sensor(s) 165 described above with reference to FIG. 1A). Memory 370 includes high-speed random access memory, such as DRAM, SRAM, DDR RAM, or other random access solid state memory devices; and optionally includes non-volatile memory, such as one or more magnetic disk storage devices, optical disk storage devices, flash memory devices, or other non-volatile solid state storage devices. Memory 370 optionally includes one or more storage devices remotely located from CPU(s) 310. In some embodiments, memory 370 stores programs, modules, and data structures analogous to the programs, modules, and data structures stored in memory 102 of portable multifunction device 100 (FIG. 1A), or a subset thereof. Furthermore, memory 370 optionally stores additional programs, modules, and data structures not present in memory 102 of portable multifunction device 100. For example, memory 370 of device 300 optionally stores drawing module 380, presentation module 382, word processing module 384, website creation module 386, disk authoring module 388, and/or spreadsheet module 390, while memory 102 of portable multifunction device 100 (FIG. 1A) optionally does not store these modules.

Each of the above-identified elements in FIG. 3A is, optionally, stored in one or more of the previously mentioned memory devices. Each of the above-identified modules corresponds to a set of instructions for performing a function described above. The above-identified modules or computer programs (e.g., sets of instructions or including instructions) need not be implemented as separate software programs (such as computer programs (e.g., including instructions)), procedures, or modules, and thus various subsets of these modules are, optionally, combined or otherwise rearranged in various embodiments. In some embodiments, memory 370 optionally stores a subset of the modules and data structures identified above. Furthermore, memory 370 optionally stores additional modules and data structures not described above.

Implementations within the scope of the present disclosure can be partially or entirely realized using a tangible computer-readable storage medium (or multiple tangible computer-readable storage media of one or more types) encoding one or more computer-readable instructions. It should be recognized that computer-readable instructions can be organized in any format, including applications, widgets, processes, software, and/or components.

Implementations within the scope of the present disclosure include a computer-readable storage medium that encodes instructions organized as an application (e.g., application 3160) that, when executed by one or more processing units, control an electronic device (e.g., device 3150) to perform the method of FIG. 3B, the method of FIG. 3C, and/or one or more other processes and/or methods described herein.

It should be recognized that application 3160 (shown in FIG. 3D) can be any suitable type of application, including, for example, one or more of: a browser application, an application that functions as an execution environment for plug-ins, widgets or other applications, a fitness application, a health application, a digital payments application, a media application, a social network application, a messaging application, and/or a maps application. In some embodiments, application 3160 is an application that is pre-installed on device 3150 at purchase (e.g., a first-party application). In some embodiments, application 3160 is an application that is provided to device 3150 via an operating system update file (e.g., a first-party application or a second-party application). In some embodiments, application 3160 is an application that is provided via an application store. In some embodiments, the application store can be an application store that is pre-installed on device 3150 at purchase (e.g., a first-party application store). In some embodiments, the application store is a third-party application store (e.g., an application store that is provided by another application store, downloaded via a network, and/or read from a storage device).

Referring to FIG. 3B and FIG. 3F, application 3160 obtains information (e.g., 3010). In some embodiments, at 3010, information is obtained from at least one hardware component of device 3150. In some embodiments, at 3010, information is obtained from at least one software module of device 3150. In some embodiments, at 3010, information is obtained from at least one hardware component external to device 3150 (e.g., a peripheral device, an accessory device, and/or a server). In some embodiments, the information obtained at 3010 includes positional information, time information, notification information, user information, environment information, electronic device state information, weather information, media information, historical information, event information, hardware information, and/or motion information. In some embodiments, in response to and/or after obtaining the information at 3010, application 3160 provides the information to a system (e.g., 3020).

In some embodiments, the system (e.g., 3110 shown in FIG. 3E) is an operating system hosted on device 3150. In some embodiments, the system (e.g., 3110 shown in FIG. 3E) is an external device (e.g., a server, a peripheral device, an accessory, and/or a personal computing device) that includes an operating system.

Referring to FIG. 3C and FIG. 3G, application 3160 obtains information (e.g., 3030). In some embodiments, the information obtained at 3030 includes positional information, time information, notification information, user information, environment information electronic device state information, weather information, media information, historical information, event information, hardware information, and/or motion information. In response to and/or after obtaining the information at 3030, application 3160 performs an operation with the information (e.g., 3040). In some embodiments, the operation performed at 3040 includes: providing a notification based on the information, sending a message based on the information, displaying the information, controlling a user interface of a fitness application based on the information, controlling a user interface of a health application based on the information, controlling a focus mode based on the information, setting a reminder based on the information, adding a calendar entry based on the information, and/or calling an API of system 3110 based on the information.

In some embodiments, one or more steps of the method of FIG. 3B and/or the method of FIG. 3C is performed in response to a trigger. In some embodiments, the trigger includes detection of an event, a notification received from system 3110, a user input, and/or a response to a call to an API provided by system 3110.

In some embodiments, the instructions of application 3160, when executed, control device 3150 to perform the method of FIG. 3B and/or the method of FIG. 3C by calling an application programming interface (API) (e.g., API 3190) provided by system 3110. In some embodiments, application 3160 performs at least a portion of the method of FIG. 3B and/or the method of FIG. 3C without calling API 3190.

In some embodiments, one or more steps of the method of FIG. 3B and/or the method of FIG. 3C includes calling an API (e.g., API 3190) using one or more parameters defined by the API. In some embodiments, the one or more parameters include a constant, a key, a data structure, an object, an object class, a variable, a data type, a pointer, an array, a list or a pointer to a function or method, and/or another way to reference a data or other item to be passed via the API.

Referring to FIG. 3D, device 3150 is illustrated. In some embodiments, device 3150 is a personal computing device, a smart phone, a smart watch, a fitness tracker, a head mounted display (HMD) device, a media device, a communal device, a speaker, a television, and/or a tablet. As illustrated in FIG. 3D, device 3150 includes application 3160 and an operating system (e.g., system 3110 shown in FIG. 3E). Application 3160 includes application implementation module 3170 and API-calling module 3180. System 3110 includes API 3190 and implementation module 3100. It should be recognized that device 3150, application 3160, and/or system 3110 can include more, fewer, and/or different components than illustrated in FIGS. 3D and 3E.

In some embodiments, application implementation module 3170 includes a set of one or more instructions corresponding to one or more operations performed by application 3160. For example, when application 3160 is a messaging application, application implementation module 3170 can include operations to receive and send messages. In some embodiments, application implementation module 3170 communicates with API-calling module 3180 to communicate with system 3110 via API 3190 (shown in FIG. 3E).

In some embodiments, API 3190 is a software module (e.g., a collection of computer-readable instructions) that provides an interface that allows a different module (e.g., API-calling module 3180) to access and/or use one or more functions, methods, procedures, data structures, classes, and/or other services provided by implementation module 3100 of system 3110. For example, API-calling module 3180 can access a feature of implementation module 3100 through one or more API calls or invocations (e.g., embodied by a function or a method call) exposed by API 3190 (e.g., a software and/or hardware module that can receive API calls, respond to API calls, and/or send API calls) and can pass data and/or control information using one or more parameters via the API calls or invocations. In some embodiments, API 3190 allows application 3160 to use a service provided by a Software Development Kit (SDK) library. In some embodiments, application 3160 incorporates a call to a function or method provided by the SDK library and provided by API 3190 or uses data types or objects defined in the SDK library and provided by API 3190. In some embodiments, API-calling module 3180 makes an API call via API 3190 to access and use a feature of implementation module 3100 that is specified by API 3190. In such embodiments, implementation module 3100 can return a value via API 3190 to API-calling module 3180 in response to the API call. The value can report to application 3160 the capabilities or state of a hardware component of device 3150, including those related to aspects such as input capabilities and state, output capabilities and state, processing capability, power state, storage capacity and state, and/or communications capability. In some embodiments, API 3190 is implemented in part by firmware, microcode, or other low level logic that executes in part on the hardware component.

In some embodiments, API 3190 allows a developer of API-calling module 3180 (which can be a third-party developer) to leverage a feature provided by implementation module 3100. In such embodiments, there can be one or more API-calling modules (e.g., including API-calling module 3180) that communicate with implementation module 3100. In some embodiments, API 3190 allows multiple API-calling modules written in different programming languages to communicate with implementation module 3100 (e.g., API 3190 can include features for translating calls and returns between implementation module 3100 and API-calling module 3180) while API 3190 is implemented in terms of a specific programming language. In some embodiments, API-calling module 3180 calls APIs from different providers such as a set of APIs from an OS provider, another set of APIs from a plug-in provider, and/or another set of APIs from another provider (e.g., the provider of a software library) or creator of the another set of APIs.

Examples of API 3190 can include one or more of: a pairing API (e.g., for establishing secure connection, e.g., with an accessory), a device detection API (e.g., for locating nearby devices, e.g., media devices and/or smartphone), a payment API, a UIKit API (e.g., for generating user interfaces), a location detection API, a locator API, a maps API, a health sensor API, a sensor API, a messaging API, a push notification API, a streaming API, a collaboration API, a video conferencing API, an application store API, an advertising services API, a web browser API (e.g., WebKit API), a vehicle API, a networking API, a WiFi API, a Bluetooth API, an NFC API, a UWB API, a fitness API, a smart home API, contact transfer API, photos API, camera API, and/or image processing API. In some embodiments, the sensor API is an API for accessing data associated with a sensor of device 3150. For example, the sensor API can provide access to raw sensor data. For another example, the sensor API can provide data derived (and/or generated) from the raw sensor data. In some embodiments, the sensor data includes temperature data, image data, video data, audio data, heart rate data, IMU (inertial measurement unit) data, lidar data, location data, GPS data, and/or camera data. In some embodiments, the sensor includes one or more of an accelerometer, temperature sensor, infrared sensor, optical sensor, heartrate sensor, barometer, gyroscope, proximity sensor, temperature sensor, and/or biometric sensor.

In some embodiments, implementation module 3100 is a system (e.g., operating system and/or server system) software module (e.g., a collection of computer-readable instructions) that is constructed to perform an operation in response to receiving an API call via API 3190. In some embodiments, implementation module 3100 is constructed to provide an API response (via API 3190) as a result of processing an API call. By way of example, implementation module 3100 and API-calling module 3180 can each be any one of an operating system, a library, a device driver, an API, an application program, or other module. It should be understood that implementation module 3100 and API-calling module 3180 can be the same or different type of module from each other. In some embodiments, implementation module 3100 is embodied at least in part in firmware, microcode, or hardware logic.

In some embodiments, implementation module 3100 returns a value through API 3190 in response to an API call from API-calling module 3180. While API 3190 defines the syntax and result of an API call (e.g., how to invoke the API call and what the API call does), API 3190 might not reveal how implementation module 3100 accomplishes the function specified by the API call. Various API calls are transferred via the one or more application programming interfaces between API-calling module 3180 and implementation module 3100. Transferring the API calls can include issuing, initiating, invoking, calling, receiving, returning, and/or responding to the function calls or messages. In other words, transferring can describe actions by either of API-calling module 3180 or implementation module 3100. In some embodiments, a function call or other invocation of API 3190 sends and/or receives one or more parameters through a parameter list or other structure.

In some embodiments, implementation module 3100 provides more than one API, each providing a different view of or with different aspects of functionality implemented by implementation module 3100. For example, one API of implementation module 3100 can provide a first set of functions and can be exposed to third-party developers, and another API of implementation module 3100 can be hidden (e.g., not exposed) and provide a subset of the first set of functions and also provide another set of functions, such as testing or debugging functions which are not in the first set of functions. In some embodiments, implementation module 3100 calls one or more other components via an underlying API and thus is both an API-calling module and an implementation module. It should be recognized that implementation module 3100 can include additional functions, methods, classes, data structures, and/or other features that are not specified through API 3190 and are not available to API-calling module 3180. It should also be recognized that API-calling module 3180 can be on the same system as implementation module 3100 or can be located remotely and access implementation module 3100 using API 3190 over a network. In some embodiments, implementation module 3100, API 3190, and/or API-calling module 3180 is stored in a machine-readable medium, which includes any mechanism for storing information in a form readable by a machine (e.g., a computer or other data processing system). For example, a machine-readable medium can include magnetic disks, optical disks, random access memory; read only memory, and/or flash memory devices.

An application programming interface (API) is an interface between a first software process and a second software process that specifies a format for communication between the first software process and the second software process. Limited APIs (e.g., private APIs or partner APIs) are APIs that are accessible to a limited set of software processes (e.g., only software processes within an operating system or only software processes that are approved to access the limited APIs). Public APIs that are accessible to a wider set of software processes. Some APIs enable software processes to communicate about or set a state of one or more input devices (e.g., one or more touch sensors, proximity sensors, visual sensors, motion/orientation sensors, pressure sensors, intensity sensors, sound sensors, wireless proximity sensors, biometric sensors, buttons, switches, rotatable elements, and/or external controllers). Some APIs enable software processes to communicate about and/or set a state of one or more output generation components (e.g., one or more audio output generation components, one or more display generation components, and/or one or more tactile output generation components). Some APIs enable particular capabilities (e.g., scrolling, handwriting, text entry, image editing, and/or image creation) to be accessed, performed, and/or used by a software process (e.g., generating outputs for use by a software process based on input from the software process). Some APIs enable content from a software process to be inserted into a template and displayed in a user interface that has a layout and/or behaviors that are specified by the template.

Many software platforms include a set of frameworks that provides the core objects and core behaviors that a software developer needs to build software applications that can be used on the software platform. Software developers use these objects to display content onscreen, to interact with that content, and to manage interactions with the software platform. Software applications rely on the set of frameworks for their basic behavior, and the set of frameworks provides many ways for the software developer to customize the behavior of the application to match the specific needs of the software application. Many of these core objects and core behaviors are accessed via an API. An API will typically specify a format for communication between software processes, including specifying and grouping available variables, functions, and protocols. An API call (sometimes referred to as an API request) will typically be sent from a sending software process to a receiving software process as a way to accomplish one or more of the following: the sending software process requesting information from the receiving software process (e.g., for the sending software process to take action on), the sending software process providing information to the receiving software process (e.g., for the receiving software process to take action on), the sending software process requesting action by the receiving software process, or the sending software process providing information to the receiving software process about action taken by the sending software process. Interaction with a device (e.g., using a user interface) will in some circumstances include the transfer and/or receipt of one or more API calls (e.g., multiple API calls) between multiple different software processes (e.g., different portions of an operating system, an application and an operating system, or different applications) via one or more APIs (e.g., via multiple different APIs). For example, when an input is detected the direct sensor data is frequently processed into one or more input events that are provided (e.g., via an API) to a receiving software process that makes some determination based on the input events, and then sends (e.g., via an API) information to a software process to perform an operation (e.g., change a device state and/or user interface) based on the determination. While a determination and an operation performed in response could be made by the same software process, alternatively the determination could be made in a first software process and relayed (e.g., via an API) to a second software process, that is different from the first software process, that causes the operation to be performed by the second software process. Alternatively, the second software process could relay instructions (e.g., via an API) to a third software process that is different from the first software process and/or the second software process to perform the operation. It should be understood that some or all user interactions with a computer system could involve one or more API calls within a step of interacting with the computer system (e.g., between different software components of the computer system or between a software component of the computer system and a software component of one or more remote computer systems). It should be understood that some or all user interactions with a computer system could involve one or more API calls between steps of interacting with the computer system (e.g., between different software components of the computer system or between a software component of the computer system and a software component of one or more remote computer systems).

In some embodiments, the application can be any suitable type of application, including, for example, one or more of: a browser application, an application that functions as an execution environment for plug-ins, widgets or other applications, a fitness application, a health application, a digital payments application, a media application, a social network application, a messaging application, and/or a maps application.

In some embodiments, the application is an application that is pre-installed on the first computer system at purchase (e.g., a first-party application). In some embodiments, the application is an application that is provided to the first computer system via an operating system update file (e.g., a first-party application). In some embodiments, the application is an application that is provided via an application store. In some embodiments, the application store is pre-installed on the first computer system at purchase (e.g., a first-party application store) and allows download of one or more applications. In some embodiments, the application store is a third-party application store (e.g., an application store that is provided by another device, downloaded via a network, and/or read from a storage device). In some embodiments, the application is a third-party application (e.g., an app that is provided by an application store, downloaded via a network, and/or read from a storage device). In some embodiments, the application controls the first computer system to perform method 700 (FIG. 7) and/or method 900 (FIG. 9) by calling an application programming interface (API) provided by the system process using one or more parameters.

In some embodiments, exemplary APIs provided by the system process include one or more of: a pairing API (e.g., for establishing secure connection, e.g., with an accessory), a device detection API (e.g., for locating nearby devices, e.g., media devices and/or smartphone), a payment API, a UIKit API (e.g., for generating user interfaces), a location detection API, a locator API, a maps API, a health sensor API, a sensor API, a messaging API, a push notification API, a streaming API, a collaboration API, a video conferencing API, an application store API, an advertising services API, a web browser API (e.g., WebKit API), a vehicle API, a networking API, a WiFi API, a Bluetooth API, an NFC API, a UWB API, a fitness API, a smart home API, contact transfer API, a photos API, a camera API, and/or an image processing API.

In some embodiments, at least one API is a software module (e.g., a collection of computer-readable instructions) that provides an interface that allows a different module (e.g., API-calling module 3180) to access and use one or more functions, methods, procedures, data structures, classes, and/or other services provided by an implementation module of the system process. The API can define one or more parameters that are passed between the API-calling module and the implementation module. In some embodiments, API 3190 defines a first API call that can be provided by API-calling module 3180. The implementation module is a system software module (e.g., a collection of computer-readable instructions) that is constructed to perform an operation in response to receiving an API call via the API. In some embodiments, the implementation module is constructed to provide an API response (via the API) as a result of processing an API call. In some embodiments, the implementation module is included in the device (e.g., 3150) that runs the application. In some embodiments, the implementation module is included in an electronic device that is separate from the device that runs the application.

Attention is now directed towards embodiments of user interfaces that are, optionally, implemented on, for example, portable multifunction device 100.

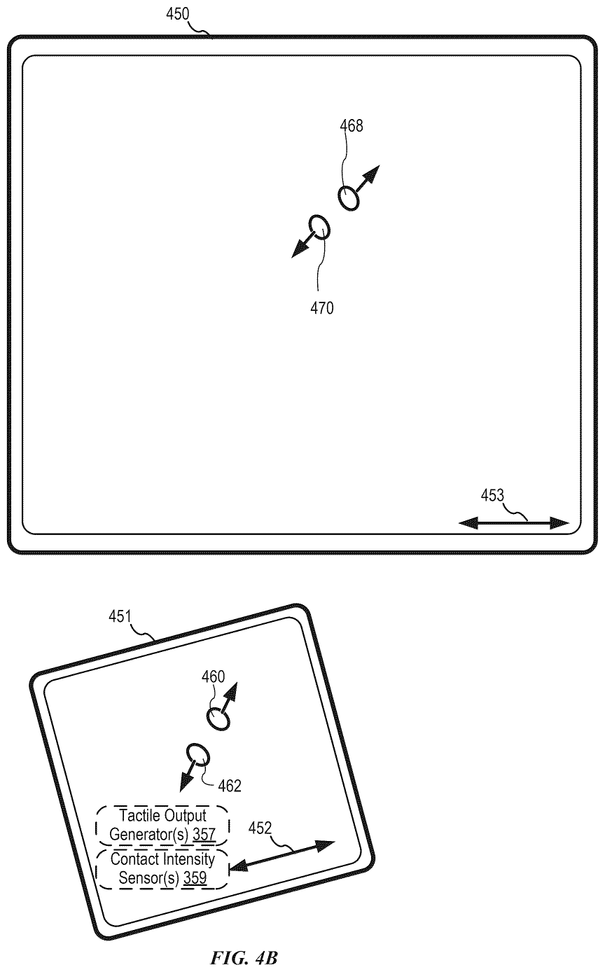

FIG. 4A illustrates an exemplary user interface for a menu of applications on portable multifunction device 100 in accordance with some embodiments. Similar user interfaces are, optionally, implemented on device 300. In some embodiments, user interface 400 includes the following elements, or a subset or superset thereof:
Signal strength indicator(s) 402 for wireless communication(s), such as cellular and Wi-Fi signals;
Time 404;
Bluetooth indicator 405;
Battery status indicator 406;
Tray 408 with icons for frequently used applications, such as:
   Icon 416 for telephone module 138, labeled “Phone,” which optionally includes an indicator 414 of the number of missed calls or voicemail messages;
   Icon 418 for e-mail client module 140, labeled “Mail,” which optionally includes an indicator 410 of the number of unread e-mails;
   Icon 420 for browser module 147, labeled “Browser;” and
   Icon 422 for video and music player module 152, also referred to as iPod (trademark of Apple Inc.) module 152, labeled “iPod;” and
Icons for other applications, such as:
   Icon 424 for IM module 141, labeled “Messages;”
   Icon 426 for calendar module 148, labeled “Calendar;”
   Icon 428 for image management module 144, labeled “Photos;”
   Icon 430 for camera module 143, labeled “Camera;”
   Icon 432 for online video module 155, labeled “Online Video;”
   Icon 434 for stocks widget 149-2, labeled “Stocks;”
   Icon 436 for map module 154, labeled “Maps;”
   Icon 438 for weather widget 149-1, labeled “Weather;”
   Icon 440 for alarm clock widget 149-4, labeled “Clock;”
   Icon 442 for workout support module 142, labeled “Workout Support;”
   Icon 444 for notes module 153, labeled “Notes;” and
   Icon 446 for a settings application or module, labeled “Settings,” which provides access to settings for device 100 and its various applications 136.

It should be noted that the icon labels illustrated in FIG. 4A are merely exemplary. For example, icon 422 for video and music player module 152 is labeled “Music” or “Music Player.” Other labels are, optionally, used for various application icons. In some embodiments, a label for a respective application icon includes a name of an application corresponding to the respective application icon. In some embodiments, a label for a particular application icon is distinct from a name of an application corresponding to the particular application icon.

FIG. 4B illustrates an exemplary user interface on a device (e.g., device 300, FIG. 3A) with a touch-sensitive surface 451 (e.g., a tablet or touchpad 355, FIG. 3A) that is separate from the display 450 (e.g., touch screen display 112). Device 300 also, optionally, includes one or more contact intensity sensors (e.g., one or more of sensors 359) for detecting intensity of contacts on touch-sensitive surface 451 and/or one or more tactile output generators 357 for generating tactile outputs for a user of device 300.

Although some of the examples that follow will be given with reference to inputs on touch screen display 112 (where the touch-sensitive surface and the display are combined), in some embodiments, the device detects inputs on a touch-sensitive surface that is separate from the display, as shown in FIG. 4B. In some embodiments, the touch-sensitive surface (e.g., touch-sensitive surface 451 in FIG. 4B) has a primary axis (e.g., 452 in FIG. 4B) that corresponds to a primary axis (e.g., 453 in FIG. 4B) on the display (e.g., display 450). In accordance with these embodiments, the device detects contacts (e.g., contact 460 and contact 462 in FIG. 4B) with the touch-sensitive surface 451 at locations that correspond to respective locations on the display (e.g., in FIG. 4B, contact 460 corresponds to 468 and contract 462 corresponds to 470). In this way, user inputs (e.g., contact 460 and contact 462, and movements thereof) detected by the device on the touch-sensitive surface (e.g., touch-sensitive surface 451 in FIG. 4B) are used by the device to manipulate the user interface on the display (e.g., 450 in FIG. 4B) of the multifunction device when the touch-sensitive surface is separate from the display. It should be understood that similar methods are, optionally, used for other user interfaces described herein.

Additionally, while the following examples are given primarily with reference to finger inputs (e.g., finger contacts, finger tap gestures, finger swipe gestures), it should be understood that, in some embodiments, one or more of the finger inputs are replaced with input from another input device (e.g., a mouse-based input or stylus input). For example, a swipe gesture is, optionally, replaced with a mouse click (e.g., instead of a contact) followed by movement of the cursor along the path of the swipe (e.g., instead of movement of the contact). As another example, a tap gesture is, optionally, replaced with a mouse click while the cursor is located over the location of the tap gesture (e.g., instead of detection of the contact followed by ceasing to detect the contact). Similarly, when multiple user inputs are simultaneously detected, it should be understood that multiple computer mice are, optionally, used simultaneously, or a mouse and finger contacts are, optionally, used simultaneously.

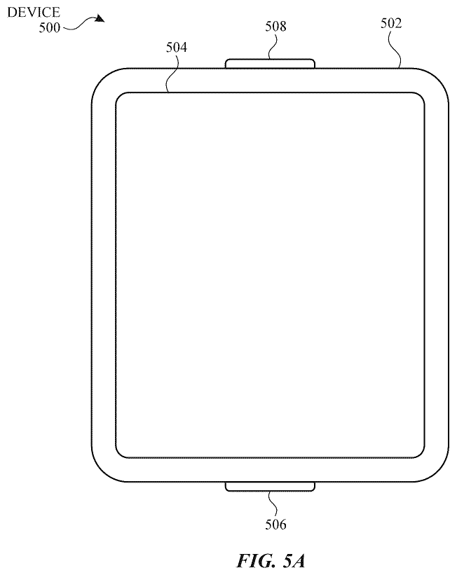

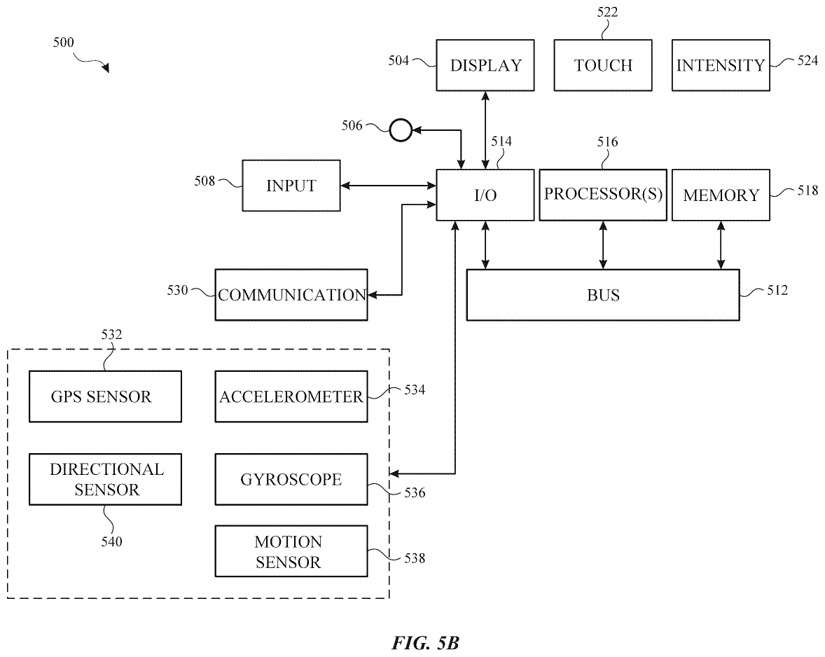

FIG. 5A illustrates exemplary personal electronic device 500. Device 500 includes body 502. In some embodiments, device 500 can include some or all of the features described with respect to devices 100 and 300 (e.g., FIGS. 1A-4B). In some embodiments, device 500 has touch-sensitive display screen 504, hereafter touch screen 504. Alternatively, or in addition to touch screen 504, device 500 has a display and a touch-sensitive surface. As with devices 100 and 300, in some embodiments, touch screen 504 (or the touch-sensitive surface) optionally includes one or more intensity sensors for detecting intensity of contacts (e.g., touches) being applied. The one or more intensity sensors of touch screen 504 (or the touch-sensitive surface) can provide output data that represents the intensity of touches. The user interface of device 500 can respond to touches based on their intensity, meaning that touches of different intensities can invoke different user interface operations on device 500.

Exemplary techniques for detecting and processing touch intensity are found, for example, in related applications: International Patent Application Serial No. PCT/US2013/040061, titled “Device, Method, and Graphical User Interface for Displaying User Interface Objects Corresponding to an Application,” filed May 8, 2013, published as WIPO Publication No. WO/2013/169849, and International Patent Application Serial No. PCT/US2013/069483, titled “Device, Method, and Graphical User Interface for Transitioning Between Touch Input to Display Output Relationships,” filed Nov. 11, 2013, published as WIPO Publication No. WO/2014/105276, each of which is hereby incorporated by reference in their entirety.

In some embodiments, device 500 has one or more input mechanisms 506 and 508.

Input mechanisms 506 and 508, if included, can be physical. Examples of physical input mechanisms include push buttons and rotatable mechanisms. In some embodiments, device 500 has one or more attachment mechanisms. Such attachment mechanisms, if included, can permit attachment of device 500 with, for example, hats, eyewear, earrings, necklaces, shirts, jackets, bracelets, watch straps, chains, trousers, belts, shoes, purses, backpacks, and so forth. These attachment mechanisms permit device 500 to be worn by a user.

FIG. 5B depicts exemplary personal electronic device 500. In some embodiments, device 500 can include some or all of the components described with respect to FIGS. 1A, 1B, and 3A. Device 500 has bus 512 that operatively couples I/O section 514 with one or more computer processors 516 and memory 518. I/O section 514 can be connected to display screen 504, which can have touch-sensitive component 522 and, optionally, intensity sensor 524 (e.g., contact intensity sensor). In addition, I/O section 514 can be connected with communication unit 530 for receiving application and operating system data, using Wi-Fi, Bluetooth, near field communication (NFC), cellular, and/or other wireless communication techniques. Device 500 can include input mechanisms 506 and/or 508. Input mechanism 506 is, optionally, a rotatable input device or a depressible and rotatable input device, for example. Input mechanism 508 is, optionally, a button, in some examples.

Input mechanism 508 is, optionally, a microphone, in some examples. Personal electronic device 500 optionally includes various sensors, such as GPS sensor 532, accelerometer 534, directional sensor 540 (e.g., compass), gyroscope 536, motion sensor 538, and/or a combination thereof, all of which can be operatively connected to I/O section 514.

Memory 518 of personal electronic device 500 can include one or more non-transitory computer-readable storage media, for storing computer-executable instructions, which, when executed by one or more computer processors 516, for example, can cause the computer processors to perform the techniques described below, including processes 700 and/or 900 (FIGS. 7 and 9). A computer-readable storage medium can be any medium that can tangibly contain or store computer-executable instructions for use by or in connection with the instruction execution system, apparatus, or device. In some examples, the storage medium is a transitory computer-readable storage medium. In some examples, the storage medium is a non-transitory computer-readable storage medium. The non-transitory computer-readable storage medium can include, but is not limited to, magnetic, optical, and/or semiconductor storages. Examples of such storage include magnetic disks, optical discs based on CD, DVD, or Blu-ray® technologies, as well as persistent solid-state memory such as flash, solid-state drives, and the like. Personal electronic device 500 is not limited to the components and configuration of FIG. 5B, but can include other or additional components in multiple configurations.

As used here, the term “affordance” refers to a user-interactive graphical user interface object that is, optionally, displayed on the display screen of devices 100, 300, and/or 500 (FIGS. 1A, 3A, and 5A-5B). For example, an image (e.g., icon), a button, and text (e.g., hyperlink) each optionally constitute an affordance.

As used herein, the term “focus selector” refers to an input element that indicates a current part of a user interface with which a user is interacting. In some implementations that include a cursor or other location marker, the cursor acts as a “focus selector” so that when an input (e.g., a press input) is detected on a touch-sensitive surface (e.g., touchpad 355 in FIG. 3A or touch-sensitive surface 451 in FIG. 4B) while the cursor is over a particular user interface element (e.g., a button, window, slider, or other user interface element), the particular user interface element is adjusted in accordance with the detected input. In some implementations that include a touch screen display (e.g., touch-sensitive display system 112 in FIG. 1A or touch screen 112 in FIG. 4A) that enables direct interaction with user interface elements on the touch screen display, a detected contact on the touch screen acts as a “focus selector” so that when an input (e.g., a press input by the contact) is detected on the touch screen display at a location of a particular user interface element (e.g., a button, window, slider, or other user interface element), the particular user interface element is adjusted in accordance with the detected input. In some implementations, focus is moved from one region of a user interface to another region of the user interface without corresponding movement of a cursor or movement of a contact on a touch screen display (e.g., by using a tab key or arrow keys to move focus from one button to another button); in these implementations, the focus selector moves in accordance with movement of focus between different regions of the user interface. Without regard to the specific form taken by the focus selector, the focus selector is generally the user interface element (or contact on a touch screen display) that is controlled by the user so as to communicate the user's intended interaction with the user interface (e.g., by indicating, to the device, the element of the user interface with which the user is intending to interact). For example, the location of a focus selector (e.g., a cursor, a contact, or a selection box) over a respective button while a press input is detected on the touch-sensitive surface (e.g., a touchpad or touch screen) will indicate that the user is intending to activate the respective button (as opposed to other user interface elements shown on a display of the device).

As used in the specification and claims, the term “characteristic intensity” of a contact refers to a characteristic of the contact based on one or more intensities of the contact. In some embodiments, the characteristic intensity is based on multiple intensity samples. The characteristic intensity is, optionally, based on a predefined number of intensity samples, or a set of intensity samples collected during a predetermined time period (e.g., 0.05, 0.1, 0.2, 0.5, 1, 2, 5, 10 seconds) relative to a predefined event (e.g., after detecting the contact, prior to detecting liftoff of the contact, before or after detecting a start of movement of the contact, prior to detecting an end of the contact, before or after detecting an increase in intensity of the contact, and/or before or after detecting a decrease in intensity of the contact). A characteristic intensity of a contact is, optionally, based on one or more of: a maximum value of the intensities of the contact, a mean value of the intensities of the contact, an average value of the intensities of the contact, a top 10 percentile value of the intensities of the contact, a value at the half maximum of the intensities of the contact, a value at the 90 percent maximum of the intensities of the contact, or the like. In some embodiments, the duration of the contact is used in determining the characteristic intensity (e.g., when the characteristic intensity is an average of the intensity of the contact over time). In some embodiments, the characteristic intensity is compared to a set of one or more intensity thresholds to determine whether an operation has been performed by a user. For example, the set of one or more intensity thresholds optionally includes a first intensity threshold and a second intensity threshold. In this example, a contact with a characteristic intensity that does not exceed the first threshold results in a first operation, a contact with a characteristic intensity that exceeds the first intensity threshold and does not exceed the second intensity threshold results in a second operation, and a contact with a characteristic intensity that exceeds the second threshold results in a third operation. In some embodiments, a comparison between the characteristic intensity and one or more thresholds is used to determine whether or not to perform one or more operations (e.g., whether to perform a respective operation or forgo performing the respective operation), rather than being used to determine whether to perform a first operation or a second operation.

As used herein, an “installed application” refers to a software application that has been downloaded onto an electronic device (e.g., devices 100, 300, and/or 500) and is ready to be launched (e.g., become opened) on the device. In some embodiments, a downloaded application becomes an installed application by way of an installation program that extracts program portions from a downloaded package and integrates the extracted portions with the operating system of the computer system.

As used herein, the terms “open application” or “executing application” refer to a software application with retained state information (e.g., as part of device/global internal state 157 and/or application internal state 192). An open or executing application is, optionally, any one of the following types of applications:
an active application, which is currently displayed on a display screen of the device that the application is being used on;
a background application (or background processes), which is not currently displayed, but one or more processes for the application are being processed by one or more processors; and
a suspended or hibernated application, which is not running, but has state information that is stored in memory (volatile and non-volatile, respectively) and that can be used to resume execution of the application.

As used herein, the term “closed application” refers to software applications without retained state information (e.g., state information for closed applications is not stored in a memory of the device). Accordingly, closing an application includes stopping and/or removing application processes for the application and removing state information for the application from the memory of the device. Generally, opening a second application while in a first application does not close the first application. When the second application is displayed and the first application ceases to be displayed, the first application becomes a background application.

In some embodiments, the computer system is in a locked state or an unlocked state. In the locked state, the computer system is powered on and operational but is prevented from performing a predefined set of operations in response to user input. The predefined set of operations optionally includes navigation between user interfaces, activation or deactivation of a predefined set of functions, and activation or deactivation of certain applications. The locked state can be used to prevent unintentional or unauthorized use of some functionality of the computer system or activation or deactivation of some functions on the computer system. In some embodiments, in the unlocked state, the computer system is powered on and operational and is not prevented from performing at least a portion of the predefined set of operations that cannot be performed while in the locked state. When the computer system is in the locked state, the computer system is said to be locked. When the computer system is in the unlocked state, the computer is said to be unlocked. In some embodiments, the computer system in the locked state optionally responds to a limited set of user inputs, including input that corresponds to an attempt to transition the computer system to the unlocked state or input that corresponds to powering the computer system off.

As described herein, content is automatically generated by one or more computers in response to a request to generate the content. The automatically-generated content is optionally generated on-device (e.g., generated at least in part by a computer system at which a request to generate the content is received) and/or generated off-device (e.g., generated at least in part by one or more nearby computers that are available via a local network or one or more computers that are available via the internet). This automatically-generated content optionally includes visual content (e.g., images, graphics, and/or video), audio content, and/or text content.

In some embodiments, novel automatically-generated content that is generated via one or more artificial intelligence (AI) processes is referred to as generative content (e.g., generative images, generative graphics, generative video, generative audio, and/or generative text). Generative content is typically generated by an AI process based on a prompt that is provided to the AI process. An AI process typically uses one or more AI models to generate an output based on an input. An AI process optionally includes one or more pre-processing steps to adjust the input before it is used by the AI model to generate an output (e.g., adjustment to a user-provided prompt, creation of a system-generated prompt, and/or AI model selection). An AI process optionally includes one or more post-processing steps to adjust the output by the AI model (e.g., passing AI model output to a different AI model, upscaling, downscaling, cropping, formatting, and/or adding or removing metadata) before the output of the AI model used for other purposes such as being provided to a different software process for further processing or being presented (e.g., visually or audibly) to a user. An AI process that generates generative content is sometimes referred to as a generative AI process.

A prompt for generating generative content can include one or more of: one or more words (e.g., a natural language prompt that is written or spoken), one or more images, one or more drawings, and/or one or more videos. AI processes can include machine learning models including neural networks. Neural networks can include transformer-based deep neural networks such as large language models (LLMs). Generative pre-trained transformer models are a type of LLM that can be effective at generating novel generative content based on a prompt. Some AI processes use a prompt that includes text to generate either different generative text, generative audio content, and/or generative visual content. Some AI processes use a prompt that includes visual content and/or an audio content to generate generative text (e.g., a transcription of audio and/or a description of the visual content). Some multi-modal AI processes use a prompt that includes multiple types of content (e.g., text, images, audio, video, and/or other sensor data) to generate generative content. A prompt sometimes also includes values for one or more parameters indicating an importance of various parts of the prompt. Some prompts include a structured set of instructions that can be understood by an AI process that include phrasing, a specified style, relevant context (e.g., starting point content and/or one or more examples), and/or a role for the AI process.

Generative content is generally based on the prompt but is not deterministically selected from pre-generated content and is, instead, generated using the prompt as a starting point. In some embodiments, pre-existing content (e.g., audio, text, and/or visual content) is used as part of the prompt for creating generative content (e.g., the pre-existing content is used as a starting point for creating the generative content). For example, a prompt could request that a block of text be summarized or rewritten in a different tone, and the output would be generative text that is summarized or written in the different tone. Similarly, a prompt could request that visual content be modified to include or exclude content specified by a prompt (e.g., removing an identified feature in the visual content, adding a feature to the visual content that is described in a prompt, changing a visual style of the visual content, and/or creating additional visual elements outside of a spatial or temporal boundary of the visual content that are based on the visual content). In some embodiments, a random or pseudo-random seed is used as part of the prompt for creating generative content (e.g., the random or pseud-random seed content is used as a starting point for creating the generative content). For example, when generating an image from a diffusion model, a random noise pattern is iteratively denoised based on the prompt to generate an image that is based on the prompt. While specific types of AI processes have been described herein, it should be understood that a variety of different AI processes could be used to generate generative content based on a prompt.

Attention is now directed towards embodiments of user interfaces (“UI”) and associated processes that are implemented on an electronic device, such as portable multifunction device 100, device 300, or device 500.

FIGS. 6A-6GG illustrate exemplary user interfaces for tracking and providing user health information, in accordance with some embodiments. The user interfaces in these figures are used to illustrate the processes described below, including the processes in FIG. 7.

FIG. 6A illustrates computer system 600, which is a smart phone with touch-sensitive display 602 and buttons 604a-604c. At FIG. 6A, computer system 600 displays home screen user interface 606. Home screen user interface 606 includes a plurality of application icons 606a-606m that are selectable to open respective applications. At FIG. 6A, computer system 600 detects user input 608, which is a touch input (e.g., a tap input) corresponding to selection of application icon 606g.

At FIG. 6B, in response to detecting user input 608, computer system 600 displays user interface 610. In some embodiments, user interface 610 is a health summary user interface that displays various health metrics pertaining to the user of computer system 600 (e.g., pertaining to a user account that is logged in on computer system 600). As will be described in greater detail below, in some embodiments, one or more regions of user interface 610 are dynamic and/or present different types of information at different times, while other regions of user interface 610 are static and/or present the same types of information at different times. In some embodiments, the dynamic regions of user interface 610 display information based on changes in user health information and/or based on new user health information that has been received since the last time the user opened and/or viewed the health application (e.g., since the last time viewed user interface 610). In some embodiments, dynamic regions of user interface 610 are dynamically generated and/or generated in response to a user request to display user interface 610 (e.g., user input 608).

In the embodiments and scenario depicted in FIG. 6B, region 612c is a static region that persistently displays the same types of user health metrics each time the user opens user interface 610. For example, in FIG. 6B, region 612c includes heart rate platter 612c-1 (e.g., which displays the user's latest heart rate reading), steps platter 612c-2 (e.g., which displays the user's steps for the current day and/or for the previous day), and calories platter 612c-3 (e.g., which displays the user's calories burned for the current day and/or for the previous day). While heart rate platter 612c-1, steps platter 612c-2, and calories platter 612c-3 may display different numerical values at different times (e.g., based on the actual measured values for these metrics and their changes throughout the day), region 612c is static in that it displays the same types of health metrics even as the user closes and re-opens user interface 610 (e.g., persistently displays heart rate, steps, and calories). In some embodiments, heart rate platter 612c-1, when selected, causes computer system 600 to display additional heart rate information (e.g., additional heart rate measurements for the user, such as heart rate measurements for the user over the last week or the last two weeks). In some embodiments, steps platter 612c-2, when selected, causes computer system 600 to display additional steps information (e.g., the number of steps taken by the user each day for the past two weeks or the past month). In some embodiments, calories platter 612c-3, when selected, causes computer system 600 to display additional calories information (e.g., the number of calories burned by the user each day for the past two weeks or the past month). In some embodiments, region 612c includes additional segments that are accessible by scrolling user interface 610 (e.g., as shown in FIG. 6G). Region 612c also includes edit option 612c-4 that, when selected, causes computer system 600 to initiate a process for modifying the types of health data that are persistently shown in region 612c.

In some embodiments, region 611 above region 612c is a dynamic region that can change as the user opens, closes, and re-opens user interface 610 at different times. In some embodiments, region 611 changes the type of health information that is displayed in region 611 based on changes in user health information and/or based on new user health information that has been received since the last time the user opened and/or viewed user interface 610. For example, in FIG. 6B, the time is 7:02 AM, and computer system 600 has received user sleep data from the previous night. The user sleep data is new user health information that has been received after user interface 610 was last displayed and/or last closed. In FIG. 6B, based on a determination that computer system 600 has received user sleep data after user interface 610 was last displayed, computer system 600 displays sleep information 612a, which includes text 612a-1 and platter 612a-2, and presents the user with information pertaining to the new sleep data.

Furthermore, in FIG. 6B, computer system 600 has also received overnight health metrics from the user from the previous night. These overnight health metrics are also new user health information that has been received after user interface 610 was last displayed and/or last closed. In FIG. 6B, based on a determination that computer system 600 has received overnight health metrics after user interface 610 was last displayed, computer system 600 displays overnight vitals information 612b, which includes text 612b-1 and platter 612b-2, and presents the user with information pertaining to the new sleep data. For example, in FIG. 6B, text 612b-1 and platter 612b-2 indicate that the user's overnight health vitals (e.g., heart rate, blood pressure, blood-oxygen level, and/or sleep duration) were within a typical range for the user except for the user's wrist temperature, which was elevated. In some embodiments, the content displayed in dynamic region 611 (e.g., text 612a-1, platter 612a, text 612b-1, and/or platter 612b) is automatically generated and/or automatically selected for display by computer system 600, for example, using one or more AI processes and/or one or more generative AI processes.

In the depicted embodiments, user interface 610 also includes option 610a. Option 610a, when selected, causes computer system 600 to display and/or open a digital assistant user interface. In some embodiments, the digital assistant user interface corresponds to a digital assistant feature of computer system 600, which provides responses in response to user-entered queries. In some embodiments, the digital assistant feature generates responses automatically, for example, using one or more AI processes and/or one or more generative AI processes. At FIG. 6B, computer system 600 detects user input 614, which is a touch input (e.g., a tap input) corresponding to selection of option 610a.

At FIG. 6C, in response to detecting user input 614, computer system 600 displays digital assistant overlay 616. In the depicted embodiments, digital assistant overlay 616 is overlaid on user interface 610. Content of user interface 610 is still displayed and/or visible to the user behind digital assistant overlay 616. However, in some embodiments, the content of user interface 610 is visually de-emphasized (e.g., displayed with decreased brightness, decreased saturation, decreased focus, and/or decreased contrast) compared to the state in FIG. 6B when digital assistant overlay 616 was not displayed. In the depicted embodiments, digital assistant overlay 616 includes text input field 618d, which prompts the user to enter a question for the digital assistant feature of computer system 600. Digital assistant overlay 616 also includes option 618e which, when selected, causes computer system 600 to activate one or more microphones and/or enter a state for receiving a spoken question input for the digital assistant feature of computer system 600. Digital assistant overlay 616 also includes question recommendations 618a-618b. In some embodiments, question recommendations 618a-618b are automatically generated by computer system 600 (e.g., using one or more AI processes and/or one or more generative AI processes). In some embodiments, question recommendations 618a-618b are automatically generated based on what content was being displayed by computer system 600 when the request to open digital assistant overlay 616 (e.g., user input 614) was detected. For example, in FIG. 6B, computer system 600 was displaying sleep information 612a and overnight vitals information 612b when user input 614 was detected and, accordingly, computer system 600 generates question recommendations 618a-618b that pertain to the user's sleep. Digital assistant overlay 616 also includes option 618c which, when selected, causes computer system 600 to generate additional question recommendations (e.g., additional question recommendations that pertain to information that was and/or is displayed in user interface 610). In some embodiments, user input outside of digital assistant overlay 616 causes computer system 600 to cease display of digital assistant overlay 616 and return to the state shown in FIG. 6B. At FIG. 6C, computer system 600 detects user input 620a, which is a touch input (e.g., a tap input) corresponding to selection of text field 618d.

At FIG. 6D, in response to detecting user input 620a, computer system 600 shifts digital assistant overlay 616 upwards and displays keyboard 618f. At FIG. 6D, computer system 600 detects user input 620b, which is one or more touch inputs (e.g., one or more tap inputs) interacting with keyboard 618f. In the depicted scenario, user input 620b corresponding to user submission of the text prompt “I've been having a lot of headaches recently, also I'm having difficulty sleeping and just feel exhausted in the morning” to the digital assistant feature of computer system 600.

At FIG. 6E, in response to detecting user input 620b, computer system 600 expands digital assistant overlay 616 into digital assistant user interface 622. In some embodiments, rather than displaying digital assistant overlay 616 in FIG. 6B, computer system 600 displays digital assistant user interface 622 in response to user input selecting option 610a. In some embodiments, digital assistant overlay 616 and digital assistant user interface 622 are the same user interface (e.g., with digital assistant user interface 622 being a version of the user interface that occupies more of display 602). In response to user input 620b, computer system 600 also displays user-entered prompt 622a within digital assistant user interface 622. In response to receiving the user-entered prompt, computer system 600 automatically generates a response to user-entered prompt 622a (e.g., using one or more AI processes and/or one or more generative AI processes), and displays response 622b within digital assistant user interface 622. Response 622b includes user health information 622b-1 through 622b-3, which presents the user with user health information that is relevant to the user-entered prompt 622a. User health information 622b-1 indicates that the user has experienced average sleep duration (for example, over the previous week, the previous two weeks, and/or the previous month). User health information 622b-2 indicates that the user has experienced below average activity levels (for example, over the previous week, the previous two weeks, and/or the previous month). User health information 622b-3 indicates that the user has not experienced any menstrual cycle deviations (for example, over the previous two months, the previous three months, and/or the previous six months). Response 622b also includes follow-up question 622b-4, which asks the user if they are currently taking any medications. Digital assistant user interface 622 displays response options 622c-1, 622c-2 that are selectable by the user to respond to follow-up question 622b-4. In this way, a user is able to interact with the digital assistant feature to get health information that is relevant to the user's questions.

FIG. 6F depicts a scenario in which it is now 4:14 pm later in the same day from FIGS. 6B-6E. At FIG. 6F, computer system 600 displays home screen user interface 606. At FIG. 6F, computer system 600 detects user input 624, which is a touch input (e.g., a tap input) corresponding to selection of application icon 606g.

At FIG. 6G, in response to detecting user input 624, computer system 600 displays user interface 610. As described above, in some embodiments, region 612c is a static region that persistently displays heart rate platter 612c-1, steps platter 612c-2, and calories platter 612c-3. However, in FIG. 6G, heart rate platter 612c-1, steps platter 612c-2, and calories platter 612c-3 have been updated with updated heart rate information, steps information, and calories information, respectively. In FIG. 6G, additional platters of static region 612c are shown, including active calories platter 612c-5, and show all data option 612d which, when selected, causes computer system 600 to display additional health data that is not shown in user interface 610.

As also described above, in some embodiments, region 611 above region 612c is a dynamic region that shows different types of user health metrics based on new user health information that has been received since the last time user interface 610 was displayed. In FIG. 6G, the last time user interface 610 was displayed was at 7:02 AM (e.g., FIG. 6B). Since that time, computer system 600 has received additional user health information about the number of steps the user has taken during the day. In FIG. 6G, based on a determination that computer system 600 has received user step data after user interface 610 was last displayed, computer system 600 displays step information 612e, which includes text 612e-1 and platter 612e-2, and presents the user with information pertaining to the new user step data. Text 612e-1 indicates that the user's number of steps for the current day is well above the user's normal or average pace for steps. Additionally, in FIG. 6G, region 611 no longer displays user sleep data 612a and/or overnight vitals data 612b that were displayed in FIG. 6B. At FIG. 6G, computer system 600 detects user input 626, which is a touch input (e.g., a tap input) corresponding to selection of option 610a.

At FIG. 6H, in response to detecting user input 626, computer system 600 displays digital assistant overlay 616 overlaid on user interface 610. As discussed previously, content of user interface 610 is still displayed and/or visible to the user behind digital assistant overlay 616, but is visually de-emphasized compared to the state in FIG. 6G. In FIG. 6G, digital assistant overlay 616 includes question recommendations 618f-618g that have been automatically generated by computer system 600 (e.g., using one or more AI processes and/or one or more generative AI processes). In some embodiments, question recommendations 618f-618g are automatically generated based on what content was being displayed by computer system 600 when the request to open digital assistant overlay 616 (e.g., user input 626) was detected. For example, in FIG. 6G, computer system 600 was displaying user step information 612e when user input 626 was detected and, accordingly, computer system 600 generates question recommendations 618e-618f that pertain to the user's steps and/or the user's health trends. At FIG. 6G, computer system 600 detects user input 628, which is a touch input (e.g., a tap input) corresponding to selection of question recommendation 618f.

At FIG. 6I, in response to detecting user input 628, computer system 600 expands digital assistant overlay 616 into digital assistant user interface 622. In response to user input 628, computer system 600 also displays question recommendation 618e as a user-submitted prompt 630a within digital assistant user interface 622. In response to receiving user-entered prompt 630a, computer system 600 automatically generates a response to user-entered prompt 630a (e.g., using one or more AI processes and/or one or more generative AI processes), and displays response 630b and response 630c within digital assistant user interface 622. Response 630b provides an automatically-generated (e.g., using one or more AI processes and/or one or more generative AI processes) summary of the user's step trends for the past month. Response 630c also displays a chart that depicts the number of steps taken by the user each day for the last month. In some embodiments, the chart in response 630c is dynamically and/or automatically generated in response to prompt 630a (e.g., using one or more AI processes and/or one or more generative AI processes). In response to prompt 630a, computer system 600 also generates and displays within user interface 622 additional question recommendations 630d-630e that pertain to prompt 630a.

FIG. 6J depicts a scenario in which it is now a different date in the future, and computer system 600 has not displayed user interface 610 in several months (e.g., the user of computer system 600 has not requested display of user interface 610 in several months). At FIG. 6J, computer system 600 displays home screen user interface 606. At FIG. 6J, computer system 600 detects user input 632, which is a touch input (e.g., a tap input) corresponding to selection of application icon 606g.

At FIG. 6K, in response to detecting user input 632, computer system 600 displays user interface 610. As described above, in some embodiments, region 612c is a static region that persistently displays heart rate platter 612c-1, steps platter 612c-2, calories platter 612c-3, and active calories platter 612c-5. In FIG. 6K, heart rate platter 612c-1, steps platter 612c-2, calories platter 612c-3, and active calories platter 612c-5 have been updated with updated heart rate information, steps information, calories information, and active calories information, respectively.

As also described above, in some embodiments, region 611 above region 612c is a dynamic region that shows different types of user health metrics based on new user health information that has been received since the last time user interface 610 was displayed. In FIG. 6K, the last time user interface 610 was displayed was on April 24 (e.g., FIG. 6G), and it is now July 3. In that time, computer system 600 has received additional user health information about the user's daily exercise minutes. In FIG. 6K, based on a determination that computer system 600 has received user exercise data after user interface 610 was last displayed, and based on a determination that user interface 610 has not been displayed in over two months, computer system 600 displays exercise trend information 612f, which includes text 612f-1 and platter 612f-2. As noted above, when the user was accessing user interface 610 multiple times in a day, the update to region 611 from FIG. 6B to FIG. 6G pertained to new data that was received during that day. However, in FIG. 6K, since the user has not accessed user interface 610 in more than two months, computer system 600 displays exercise trends over a greater duration of time, e.g., from the last three months. Text 612f-1 indicates that, over the last three months, the user's daily exercise minutes have steadily increased, and platter 612f-2 includes a chart that depicts the user's daily exercise minutes over the last three months. In some embodiments, text 612f-1 and/or the chart in platter 612f-2 are dynamically generated by computer system (e.g., using one or more AI processes and/or one or more generative AI processes). At FIG. 6K, computer system 600 detects user input 634, which is a touch input (e.g., a tap input) corresponding to selection of option 610a.

At FIG. 6L, in response to detecting user input 634, computer system 600 displays digital assistant overlay 616 overlaid on user interface 610. As discussed previously, content of user interface 610 is still displayed and/or visible to the user behind digital assistant overlay 616, but is visually de-emphasized compared to the state in FIG. 6K. In FIG. 6L, digital assistant overlay 616 includes question recommendations 618h-618i that have been automatically generated by computer system 600 (e.g., using one or more AI processes and/or one or more generative AI processes). In some embodiments, question recommendations 618h-618i are automatically generated based on what content was being displayed by computer system 600 when the request to open digital assistant overlay 616 (e.g., user input 634) was detected. For example, in FIG. 6L, computer system 600 was displaying exercise trend information 612f when user input 634 was detected and, accordingly, computer system 600 generates question recommendations 618h-618i that pertain to exercise and/or other health trends from the last three months. At FIG. 6L, computer system 600 detects user input 636, which is a touch input (e.g., a tap input) corresponding to selection of question recommendation 618h.

At FIG. 6M, in response to detecting user input 636, computer system 600 expands digital assistant overlay 616 into digital assistant user interface 622. In response to user input 636, computer system 600 also displays question recommendation 618h as a user-submitted prompt 638a within digital assistant user interface 622. In response to receiving user-entered prompt 638a, computer system 600 automatically generates a response to user-entered prompt 638a (e.g., using one or more AI processes and/or one or more generative AI processes), and displays response 638b and response 638c within digital assistant user interface 622. Computer system 600 generates response 638b by accessing the user's workout history to note that the user's latest workouts indicate that the user is focusing on improving upper body strength. Response 638c displays an automatically-generated follow up question that will assist computer system 600 in selecting workout recommendations for the user in response to prompt 638a. Computer system 600 also generates and displays, within user interface 622, response options 640a-640b that the user can select to respond to follow-up question 638c. The user can also manually enter a response using text field 622d. At FIG. 6M, computer system 600 detects user input 641, which is a touch input (e.g., a tap input) corresponding to selection of response option 640b.

At FIG. 6N, in response to user input 641, computer system 600 displays user-entered response 638d in user interface 622, and also generates and displays response 638e and workout recommendations 638f-638f. Workout recommendations 638f-638g are automatically selected by computer system 600 (e.g., from a set of available workouts) based on the user's workout history (e.g., user's workout history indicating that the user has been focusing on upper body strength), and based on user input 641 specifying that the user would like to do a 30-minute workout. In some embodiments, workout recommendation 638f, when selected, causes computer system 600 to initiate playback of a first workout (e.g., initiate playback of video and/or audio guiding the user through a 30-minute full body flow yoga workout); and workout recommendation 638g, when selected, causes computer system 600 to initiate playback of a second workout (e.g., initiate playback of video and/or audio guiding the user through a 30-minute upper body strength workout). Additionally, in response to user input 641, computer system 600 also generates and displays follow-up question recommendations 640d-640e. At FIG. 6N, computer system 600 detects user input 642, which is a touch input corresponding to selection of text field 622d.

At FIG. 60, in response to detecting user input 642, computer system 600 displays keyboard 644. At FIG. 60, computer system 600 detects user input 646, which is one or more user inputs interacting with keyboard 644 to enter the user query “Can you log my meal into the health app?”

At FIG. 6P, in response to detecting user input 646, computer system 600 displays the user-entered query 638h in user interface 622. At FIG. 6Q, in response to user input 646 and/or in response to receiving user-entered query 638h, computer system 600 generates and display response 638i. Response 638i asks the user whether they would like to log a meal by taking a photo, entering a previously-taken photo, or describing the meal via text. Computer system 600 also generates and displays response options 640g-640i which are selectable by a user to enter meal information by taking a photo, entering a previously-taken photo, or describing the meal via text, respectively. At FIG. 6Q, computer system 600 detects user input 648, which is a touch input (e.g., a tap input) corresponding to selection of response option 640g.

At FIG. 6R, in response to detecting user input 648, computer system 600 displays camera user interface 650. Camera user interface 650 includes preview region 650a, which displays visual information being captured by one or more cameras of computer system 600 and provides the user with a preview of what their photo and/or video will look like when captured. Camera user interface 650 also includes shutter button 650b and options 650c-650n. Shutter button 650b, when selected, causes computer system 600 to capture a photograph. Option 650c, when selected, causes computer system 600 to switch between a front-facing camera of computer system 600 and a rear-facing camera of computer system 600. Option 650d, when selected, causes computer system 600 to display a photo library user interface. Option 650e, when selected, causes computer system 600 to switch from a currently selected photo mode to a cinematic video mode. Option 650f, when selected, causes computer system 600 to switch from a currently selected photo mode to a video mode. Option 650g is currently selected, and corresponds to a photo mode. Option 650h, when selected, causes computer system 600 to switch from a currently selected photo mode to a portrait photo mode. Option 650i, when selected, causes computer system 600 to switch from a currently selected photo mode to a panoramic image mode. Option 650j, when selected, causes computer system 600 to zoom out (e.g., switching from a first camera with a first amount of zoom to a second camera with less zoom; or digitally zoom out). Option 650k is currently selected, and corresponds to a first level of zoom. Option 650l, when selected, causes computer system 600 to zoom in by two times (e.g., switching from a first camera with a first amount of zoom to a third camera with twice the zoom; or digitally zoom in). Option 650m, when selected, causes computer system 600 to zoom in by five times (e.g., switching from a first camera with a first amount of zoom to a fourth camera with five times the zoom; or digitally zoom in). Option 650n, when selected, causes computer system 600 to enable or disable a flash feature. At FIG. 6R, computer system 600 detects user input 652, which is a touch input (e.g., a tap input) corresponding to selection of shutter button 650b.

At FIG. 6S, in response to user input 652, computer system 600 captures the photograph. Furthermore, in response to user input 652, computer system 600 re-displays user interface 622 and displays the photograph within user interface 622 as user-inputted message 638j. In response to receiving the photograph, computer system 600 automatically identifies food in the photograph (e.g., using one or more AI processes and/or generative AI processes), calculates nutrition information for the identified food, and generates and displays response 638k, which identifies the food that is depicted in the user-entered photograph and provides the nutrition information for the identified food. Additionally, in FIG. 6S, in response to user input 652, computer system 600 generates and displays question options 640j-640k that pertain to user-inputted messages 638h and 638j and are selectable by a user to submit additional questions to the digital assistant feature of computer system 600.

FIGS. 6T-6GG illustrates various features and embodiments in which computer system 600 displays and/or provides access to laboratory test results and/or laboratory testing services within user interface 610.

At FIG. 6T, computer system 600 displays user interface 610, various embodiments of which were described above, for example, with reference to FIG. 6B. However, in FIG. 6T, computer system 600 displays reminder 654 within user interface 610. Reminder 654 indicates that the user of computer system 600 has not received laboratory test results and/or has not scheduled laboratory test results for a threshold duration of time. In some embodiments, computer system 600 displays reminder 654 within user interface 610 based on a determination that the user of computer system 600 has not received and/or has not scheduled laboratory test results for a threshold duration of time. In some embodiments, computer system 600 determines one or more recommended laboratory tests based on health data associated with the user, biometric sensor information collected from the user, and/or other biometric information collected from the user. In some embodiments, computer system 600 displays reminder 654 (e.g., within user interface 610 and/or outside of user interface 610) based on a determination that one or more laboratory tests are recommended for the user (e.g., based on biometric information collected from the user and/or based on health data associated with the user). Reminder 654 is displayed with option 654a which, when selected, causes computer system 600 to cease displaying reminder 654 within user interface 610. At FIG. 6T, computer system 600 detects user input 655, which is a selection input (e.g., in FIG. 6T, a touch input and/or a tap input) corresponding to selection of reminder 654.

At FIG. 6U, in response to detecting user input 655, computer system 600 displays user interface 656. In the depicted embodiment, user interface 656 includes options 658a-658h. Option 658a, when selected, causes computer system 600 to cease display user interface 656 and, for example, return to the state shown in FIG. 6T. Similarly, option 658b, when selected, causes computer system 600 to cease displaying user interface 656 and, for example, return to the state shown in FIG. 6T.

Options 658c-658f allow the user to select which laboratory tests the user would like to order and/or schedule. Option 658c, when selected, causes computer system 600 to include or exclude a lipids test to the user's request. In FIG. 6U, option 658c is shown with a check mark to indicate that a lipids test is currently included in the user's request. In the state shown in FIG. 6U, selection of option 658c causes computer system 600 to remove the lipids test from the user's request (and, for example, causes computer system 600 to remove the check mark from option 658c). In FIG. 6U, option 658c is shown with price information indicating the cost of ordering and/or performing the lipids test. Option 658d, when selected, causes computer system 600 to include or exclude a metabolic panel to the user's request. In FIG. 6U, option 658d is shown with a check mark to indicate that a metabolic panel is currently included in the user's request. In the state shown in FIG. 6U, selection of option 658d causes computer system 600 to remove the metabolic panel from the user's request (and, for example, causes computer system 600 to remove the check mark from option 658d). In FIG. 6U, option 658d is shown with price information indicating the cost of ordering and/or performing the metabolic panel. In some embodiments, the pricing information provided corresponds to a price for ordering the test with a third-party testing service and/or a third-party testing entity. Option 658e, when selected, causes computer system 600 to include or exclude a complete blood count to the user's request. In FIG. 6U, option 658e is shown with a check mark to indicate that a complete blood count is currently included in the user's request. In the state shown in FIG. 6U, selection of option 658e causes computer system 600 to remove the complete blood count from the user's request (and, for example, causes computer system 600 to remove the check mark from option 658e). In FIG. 6U, option 658e is shown with price information indicating the cost of ordering and/or performing the complete blood count. Option 658f, when selected, causes computer system 600 to include or exclude a HBA1C test to the user's request. In FIG. 6U, option 658f is shown with a check mark to indicate that an HBA1C is currently included in the user's request. In the state shown in FIG. 6U, selection of option 658f causes computer system 600 to remove the HBA1C test from the user's request (and, for example, causes computer system 600 to remove the check mark from option 658f). In FIG. 6U, option 658f is shown with price information indicating the cost of ordering and/or performing the HBA1C test.

Option 658g, when selected, causes computer system 600 to proceed with a process for scheduling and/or ordering laboratory tests based on the tests selected in options 658c-658f, as will be described in greater detail below. Option 658h, when selected, causes computer system 600 to display a user interface and/or proceed with an alternative process in which the user of computer system 600 is able to manually enter laboratory test results that the user of computer system 600 has received. At FIG. 6U, while options 658c-658f are all in a selected and/or included state, computer system 600 detects user input 659 (e.g., in FIG. 6U, a touch input and/or a tap input) corresponding to selection of option 658g.

At FIG. 6V, in response to detecting user input 659, computer system 600 displays user interface 660. In the depicted embodiment, user interface 660 includes options 662a-662d. Option 662a, when selected, causes computer system 600 to cease display of user interface 660 and, for example, return to the state shown in FIG. 6U. Option 662b, when selected, causes computer system 600 to cease display of user interface 600 and, for example, return to the state shown in FIG. 6T. Option 662c, when selected, causes computer system 600 to proceed with a process for scheduling and/or ordering the selected laboratory tests with a laboratory testing service and/or a laboratory testing entity. In some embodiments, the laboratory testing service and/or the laboratory testing entity is a third-party service that is separate from the entity that provides and/or generates user interface 610. Option 662d, when selected, causes computer system 600 to generate and/or output a form that is usable by the user of computer system 600 to order the selected laboratory tests from a different entity different from the laboratory testing service associated with option 662c. At FIG. 6V, computer system 600 detects user input 663, which is a selection input (e.g., in FIG. 6V, a touch input and/or a tap input) corresponding to selection of option 662c.

At FIG. 6W, in response to detecting user input 663, computer system 600 displays user interface 664. In the depicted embodiment, user interface 664 includes options 664a-664b. Option 664a, when selected, causes computer system 600 to cease display of user interface 664 and, for example, return to the state shown in FIG. 6V. Option 664b, when selected, causes computer system 600 to cease display of user interface 664 and, for example, return to the state shown in FIG. 6T. User interface 664 also includes location indication 667, map 668, location options 670a-670b, and option 664c. Location indication 667 indicates a currently selected location (e.g., geographic location, zip code, and/or city). Location options 670a-670b indicates one or more laboratory testing locations at which the user can schedule laboratory tests within the currently selected location. In some embodiments, location options 670a-670b are scrollable (e.g., horizontally in FIG. 6W) to reveal additional location options within and/or proximate the currently selected location. In some embodiments, the currently selected location is selected automatically based on the user's current location and/or the current location of computer system 600. In some embodiments, the user of computer system 600 is able to specify and/or enter a currently selected location (e.g., able to specify and/or enter a location to perform a search for testing locations). Map 668 displays a geographic map of the currently selected location and/or corresponding to the currently selected location, and displays location indications 668a-668c which indicate the locations of different testing centers within the displayed map region. For example, in some embodiments, indication 668a indicates the location of the testing location associated with location option 670a, and indication 668b indicates the location of the testing location associated with location option 670b. At FIG. 6W, computer system 600 detects user input 671a, which is a selection input (e.g., in FIG. 6W, a touch input and/or a tap input) corresponding to selection of location option 670a. At FIG. 6X, in response to detecting user input 671a, computer system 600 displays location option 670a with a bolded border to indicate that location option 670a is currently selected. Additionally, in response to detecting user input 671a, computer system 600 also displays indication 668a with a bolded border to indicate the location of the currently selected location option 670a within map 668. At FIG. 6X, while location option 670a is selected, computer system 600 detects user input 671b, which is a selection input (e.g., in FIG. 6X, a touch input and/or a tap input) corresponding to selection of option 664c. In some embodiments, user input 671b while location option 670a is selected corresponds to a user request to proceed with the process for scheduling one or more laboratory test results at the selected testing location.

At FIG. 6Y, in response to detecting user input 671b, computer system 600 displays user interface 672. In the depicted embodiment, user interface 672 includes options 672a, 672b, and 672c. Option 672a, when selected, causes computer system 600 to cease display of user interface 672 and, for example, return to the state shown in FIG. 6W or 6X. Option 672b, when selected, causes computer system 600 to cease display of user interface 672 and, for example, return to the state shown in FIG. 6T. Option 672c, when selected, causes computer system 600 to proceed with the process for scheduling one or more laboratory tests, as will be described in greater detail below. User interface 672 also displays one or more date options 674a and time options 674b with available appointment times for the selected testing location. In the depicted embodiment, date options 674a are selectable by a user to change the date of the requested testing appointment, and time options 674b are shown to indicate the available appointment times for the selected testing location and the selected date. In FIG. 6Y, Tuesday May 30 is selected within date options 674a, and time options 674b indicate the available appointment times on Tuesday May 30 at the selected testing location. At FIG. 6Y, computer system 600 detects user input 675a, which is a selection input (e.g., in FIG. 6Y, a touch input and/or a tap input) corresponding to selection of time option 674b-1. At FIG. 6Z, in response to detecting user input 675a, computer system 600 displays time option 674b-1 with a bolded border to indicate that time option 674b-1 is currently selected. At FIG. 6Z, computer system 600 detects user input 675b, which is a selection input (e.g., in FIG. 6Z, a touch input and/or a tap input) corresponding to selection of option 672c.

At FIG. 6AA, in response to detecting user input 675b, computer system 600 displays user interface 676. In the depicted embodiments, user interface 676 includes options 676a-676d, as well as options 678a-678e. Option 676a, when selected, causes computer system 600 to cease display of user interface 676 and, for example, return to the state shown in FIG. 6Y or 6Z. Option 676b, when selected, cause computer system 600 to cease display of user interface 676 and, for example, return to the state shown in FIG. 6T. Option 676c, when selected, causes computer system 600 to proceed with the process for scheduling and/or ordering one or more laboratory tests, as will be described in greater detail below. Option 676d, when selected, causes computer system 600 to initiate a process for the user to log into a user account with the third-party laboratory testing service. Options 678a-678e, when selected, cause computer system 600 to display a keyboard, a keypad, and/or selectable options for the user of computer system 600 to enter their personal information (e.g., name, phone number, email address, date of birth, and sex, respectively). In some embodiments, options 678a-678e are automatically pre-filled based on user information that is available to and/or accessible by computer system 600. At FIG. 6AA, computer system 600 detects user input 679, which is a selection input (e.g., in FIG. 6AA, a touch input and/or a tap input) corresponding to selection of option 676c.

At FIG. 6BB, in response to detecting user input 679, computer system 600 displays user interface 680. User interface 680 is a payment user interface that displays indication 680e-1 indicative of the tests being ordered, indication 680e-2 indicative of the date and time of the requested testing appointment, and indication 680e-3 indicative of the total price for ordering the selected tests. In the depicted embodiment, user interface 680 includes options 680a-680d.

Option 680a, when selected, causes computer system 600 to cease display of user interface 680 and, for example, return to the state shown in FIG. 6AA. Option 680b, when selected, causes computer system 600 to cease display of user interface 680 and, for example, return to the state shown in FIG. 6T or the state shown in FIG. 6AA. Option 680c, when selected, causes computer system 600 to initiate a process for payment using a digital wallet stored on computer system 600 and/or accessible to computer system 600. Option 680d, when selected, causes computer system 600 to display a user interface in which the user is able to manually enter credit card information to pay for the requested laboratory tests. At FIG. 6BB, computer system 600 detects user input 681, which is a selection input (e.g., in FIG. 6BB, a touch input and/or a tap input) corresponding to selection of option 680c.

At FIG. 6CC, in response to detecting user input 681, computer system 600 displays payment overlay 682. In the depicted embodiment, payment overlay 682 includes option 682a, indication 682b, option 682c, option 682d, indication 682e, and instruction 682f. Option 682a, when selected, causes computer system 600 to cease display of payment overlay 682 and, for example, return to the state shown in FIG. 6BB. Indication 682b indicates a currently selected payment credential (e.g., a credit card that is stored in a digital wallet) that will be used for making the payment. Option 682c, when selected, causes computer system 600 to display one or more payment credential options of the digital wallet that are selectable by the user to change the payment credential to be used for payment. Option 682d indicates currently selected contact information for, for example, sending payment confirmation information and/or appointment confirmation information. Option 682d, when selected, causes computer system 600 to display a user interface for the user to select and/or enter different contact information for, for example, receiving payment confirmation information and/or appointment confirmation information. Indication 682e indicates the total amount to be paid. Instruction 682f instructs the user to confirm the payment using button 604c. At FIG. 6CC, computer system 600 detects user input 683, which, in FIG. 6CC, is one or more presses of button 604c.

At FIG. 6DD, in response to detecting user input 683, computer system 600 initiates payment of the request amount using the selected payment credential, and displays user interface 684. User interface 684 indicates that payment has successfully been made and the request testing appointment has been made with the laboratory testing service. In FIG. 6DD, user interface 684 includes: date and time information 684a indicating the date and time of the scheduled appointment; location information 684b indicating the location of the testing center for the appointment; test information 684c indicating the laboratory tests that have been scheduled; and options 684d-684e. Option 684d, when selected, causes computer system 600 to cease display of user interface 684. Option 684e, when selected, causes computer system 600 to initiate a process for adding a calendar entry corresponding to the scheduled appointment within a calendar application. At FIG. 6DD, computer system 600 detects user input 685, which is a selection input (e.g., in FIG. 6DD, a touch input and/or a tap input) corresponding to selection of option 684d.

At FIG. 6EE, in response to user input 685, computer system 600 displays user interface 686. In the depicted embodiment, user interface 686 includes option 686a, appointment information 688, and options 688a-688c. Option 686a, when selected, causes computer system 600 to cease display of user interface 686. Appointment information 688 displays information corresponding to the scheduled laboratory testing appointment, including the date, time, and location of the appointment, and the laboratory tests requested and/or scheduled. Option 688a, when selected, causes computer system 600 to initiate a process for canceling the laboratory testing appointment. Option 688b, when selected, causes computer system 600 to initiate a process for rescheduling the laboratory testing appointment. Option 688c, when selected, causes computer system 600 to display a payment receipt corresponding to the laboratory testing appointment.

At FIG. 6FF, computer system 600 displays user interface 610, various embodiments of which have been described above. For example, in some embodiments, computer system 600 displays user interface 610 in response to user input 608 in FIG. 6A. However, in FIG. 6FF, user interface 610 is being displayed by computer system 600 after the user has completed their laboratory testing appointment and after computer system 600 has received laboratory testing results (e.g., from a third-party laboratory testing service that performed the laboratory testing). At FIG. 6FF, based on a determination that computer system 600 has received laboratory testing results after user interface 610 was last displayed, computer system 600 displays lab results indication 689a within region 611 of user interface 610. As discussed above, dynamically modifying the order and/or substance of information presented within user interface 610 based on new information that has been received since user interface 610 was last displayed surfaces relevant, updated, and/or important information for the user of computer system 600. Lab results indication 689a indicates that laboratory testing results have been received and are available for viewing by the user. In some embodiments, lab results indication 689a includes at least a subset of the lab results received from the laboratory testing service. At FIG. 6FF, computer system 600 detects user input 689b, which is a selection input (e.g., in FIG. 6FF, a touch input and/or a tap input) corresponding to selection of lab results indication 689a.

At FIG. 6GG, in response to detecting user input 689b, computer system 600 displays user interface 690. User interface 690 displays laboratory results received from the laboratory testing service. In some embodiments, user interface 690 displays additional laboratory results that are not displayed in lab results indication 689a and/or user interface 610. In the depicted embodiment, user interface 690 includes option 690a and lab results information 692. Option 690a, when selected, causes computer system 600 to cease display of user interface 690 and, for example, return to the state shown in FIG. 6FF. Lab results information 692 displays laboratory testing results received from the laboratory testing service. For example, in FIG. 6GG, lab results information 692 includes lipids information 692a, HBA1C information 692b, and thyroid function information 692c. In FIG. 6GG, lipids information 692a indicates that the user's lipid profile is outside of normal range and requires attention. In some embodiments, lipids information 692a displays the user's actual lipid level test results (e.g., total cholesterol level, HDL level, LDL level, and/or triglyceride levels). HBA1C information 692b and thyroid function information 692c indicate that the user's HBA1C levels and thyroid function levels are within normal range. In some embodiments, HBA1C information 692b displays the user's tested HBA1C levels and thyroid function information 692c displays the user's thyroid function test results. In some embodiments, user interface 690 displays additional test results that are not shown in FIG. 6GG (e.g., additional blood count testing results and/or levels, metabolic panel testing results and/or levels, HBA1C testing results and/or levels, and/or lipid panel testing results and/or levels).

FIG. 7 is a flow diagram illustrating a method for tracking and providing user health information using a computer system in accordance with some embodiments. Method 700 is performed at a computer system (e.g., 100, 300, 500, and/or 600) that is in communication with one or more display generation components (e.g., 602) (e.g., a display, a touch-sensitive display, and/or a display controller) and one or more input devices (e.g., 602, 604a, 604b, and/or 604c) (e.g., a touch-sensitive surface, a touch-sensitive display, a button, a rotatable input mechanism, a depressible and rotatable input mechanism, a camera, an accelerometer, an inertial measurement unit (IMU), a heartrate sensor, a body temperature sensor, and/or a blood-oxygen level sensor). Some operations in method 700 are, optionally, combined, the orders of some operations are, optionally, changed, and some operations are, optionally, omitted.

As described below, method 700 provides an intuitive way for tracking and providing user health information. The method reduces the cognitive burden on a user for accessing health information, thereby creating a more efficient human-machine interface. For battery-operated computing devices, enabling a user to access health information faster and more efficiently conserves power and increases the time between battery charges.

The computer system (e.g., 600) detects (702), via the one or more input devices (e.g., 602), a first user input (e.g., 608, 624, and/or 632) (e.g., one or more user inputs) (e.g., one or more touch inputs, one or more gesture inputs, one or more hardware control inputs, and/or one or more spoken inputs) corresponding to a user request to display a health summary user interface (e.g., 610). In response to detecting the first user input (704), the computer system displays (706), via the one or more display generation components, the health summary user interface (e.g., 610), including: in accordance with a determination that a first set of health information has been received for a user of the computer system (e.g., biometric information; information pertaining to the physical health of the user; and/or information pertaining to physical metrics of the user) since the health summary user interface was last displayed (e.g., since the health summary user interface was last displayed by the computer system) (e.g., since a health application that displays and/or generates the health summary user interface was last opened) (708), displaying (710), within a first region (e.g., 611) of the health summary user interface, a first set of information corresponding to a first type of health data (e.g., a first category of health data) based on the first set of health information (e.g., FIG. 6B); and in accordance with a determination that a second set of health information different from the first set of health information has been received for the user of the computer system (e.g., biometric information; information pertaining to the physical health of the user; and/or information pertaining to physical metrics of the user) since the health summary user interface was last displayed (712), displaying (714), within the first region (e.g., 611) of the health summary user interface, a second set of information corresponding to a second type of health data (e.g., a second category of health data) based on the second set of health information, wherein the second set of information is different from the first set of information, and the second type of health data is different from the first type of health data (e.g., FIG. 6G and/or FIG. 6K). In some embodiments, the second set of information does not include information corresponding to the first type of health data and/or the first set of information does not include information corresponding to the second type of health data. In some embodiments, the health summary user interface displays one or more health metrics corresponding to a user of the computer system. In some embodiments, the health summary user interface displays one or more daily health metrics corresponding to the user of the computer system and corresponding to a current day (e.g., measured during the current day, measured in the previous evening leading into the current day, and/or that excludes health data collected and/or measured during previous days). In some embodiments, the health summary user interface displays a plurality of distinct regions that correspond to different types of health data. For example, in some embodiments, the health summary user interface includes a first region (e.g., 612c-1) that corresponds to a first type of health data (e.g., displays a first set of health data that corresponds to the first type of health data) and a second region (e.g., 612c-2) that corresponds to a second type of health data (e.g., displays a second set of health data that corresponds to the second type of health data and, optionally, does not correspond to the first type of health data) different from the first type of health data. In some embodiments, the first region, when selected, causes the computer system to display a first user interface that displays additional information corresponding to the first type of health data that was not displayed in the health summary user interface. In some embodiments, the second region, when selected, causes the computer system to display a second user interface that displays additional information corresponding to the second type of health data that was not displayed in the health summary user interface. Presenting different content within the health summary user interface and/or re-ordering information within the health summary user interface based on what information has been received since the last time the health summary user interface was displayed allows the user to access the most relevant information for the user with fewer and/or no additional user inputs. Furthermore, doing so also enhances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently.

In some embodiments, displaying the health summary user interface (e.g., 610) further comprises: in accordance with a determination that the first set of health information has been received for a user of the computer system (e.g., biometric information; information pertaining to the physical health of the user; and/or information pertaining to physical metrics of the user) since the health summary user interface was last displayed (e.g., since the health summary user interface was last displayed by the computer system) (e.g., since a health application that displays and/or generates the health summary user interface was last opened), displaying, within a second region of the health summary user interface different from the first region, a third set of information corresponding to a third type of health data (e.g., a third category of health data) based on the first set of health information; and in accordance with a determination that the second set of health information different from the first set of health information has been received for the user of the computer system (e.g., biometric information; information pertaining to the physical health of the user; and/or information pertaining to physical metrics of the user) since the health summary user interface was last displayed, displaying, within the second region of the health summary user interface, a fourth set of information corresponding to a fourth type of health data (e.g., a fourth category of health data) based on the second set of health information, wherein the fourth set of information is different from the third set of information, and the fourth type of health data is different from the third type of health data (e.g., in some embodiments, region 611 includes both a first type of health data and a second type of health data, as in FIG. 6B (e.g., 612a and 612b), and in some embodiments, both of these types of health data are replaced with different types of health data at a different time (e.g., in FIGS. 6G, 612a is replaced by 612e and 612b is, in some embodiments, replaced by a different type of health data)). In some embodiments, the second set of information does not include information corresponding to the third type of health data and/or the first set of information does not include information corresponding to the fourth type of health data. Presenting different content within the health summary user interface and/or re-ordering information within the health summary user interface based on what information has been received since the last time the health summary user interface was displayed allows the user to access the most relevant information for the user with fewer and/or no additional user inputs. Furthermore, doing so also enhances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently.

In some embodiments, the health summary user interface (e.g., 610) includes an agent user interface object (e.g., 610a) (e.g., a displayed user interface object and/or a selectable user interface object) that, when selected, causes the computer system (e.g., 600) to display an agent user interface (e.g., 616 and/or 622), wherein the agent user interface includes one or visual prompts (e.g., 618f, 618g, and/or 618d) (e.g., displayed prompts, displayed instructions, displayed user interface objects) that prompt the user of the computer system to ask one or more questions (e.g., one or more visual prompts that instruct the user to provide one or more questions (e.g., to type in one or more questions; to select one or more questions; and/or to speak one or more questions)). In some embodiments, while displaying the health summary user interface (e.g., 610), the computer system detects, via the one or more input devices, one or more user inputs (e.g., 614, 626, and/or 634) (e.g., one or more touch inputs, one or more gesture inputs, one or more hardware control inputs, and/or one or more spoken inputs) corresponding to selection of the agent user interface object (e.g., 610a). In response to detecting the one or more user inputs corresponding to selection of the agent user interface object, the computer system displays, via the one or more display generation component, the agent user interface (e.g., 616 and/or 622). Displaying a user interface object that is selectable to display an agent user interface allows the user to perform these operations with fewer inputs. Furthermore, doing so also enhances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently.

In some embodiments, displaying the agent user interface (e.g., 616 and/or 622) comprises concurrently displaying, within the agent user interface: a first visual prompt (e.g., 618d and/or 622d) prompting the user to ask one or more questions (e.g., a text-entry field for entering one or more questions; and/or written instructions for the user to enter one or more questions and/or to speak one or more questions); and one or more automatically-generated (e.g., using an AI process or a generative AI process) question recommendations (e.g., 618a, 618b, 618f, 618g, 630d, 630e, 618h, and/or 618i) (e.g., one or more automatically-generated question recommendations that are selectable by the user). Automatically generating question recommendations for a user allows a user to ask and/or submit these questions with fewer inputs. Furthermore, doing so also enhances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently.

In some embodiments, displaying the one or more automatically-generated question recommendations comprises: in accordance with a determination that the first set of information corresponding to the first type of health data was displayed when the one or more user inputs corresponding to selection of the agent user interface object were detected (e.g., FIG. 6B) (and, optionally, the second set of information corresponding to the second type of health data was not displayed when the one or more user inputs corresponding to selection of the agent user interface object were detected), displaying a first set of one or more automatically-generated question recommendations that are generated (e.g., using an AI process or a generative AI process) based on the first set of information (e.g., 618a and 618b in FIG. 6C) (e.g., one or more automatically-generated question recommendations that pertain to and/or are relevant to the first set of information) (e.g., in some embodiments, without displaying one or more automatically-generated question recommendations that are generated based on the second set of information); and in accordance with a determination that the second set of information corresponding to the second type of health data was displayed when the one or more user inputs corresponding to selection of the agent user interface object were detected (e.g., FIG. 6G) (and, optionally, the first set of information corresponding to the first type of health data was not displayed when the one or more user inputs corresponding to selection of the agent user interface object were detected), displaying a second set of one or more automatically-generated question recommendations that are generated (e.g., using an AI process or a generative AI process) based on the second set of information (e.g., 618f-618f in FIG. 6H) (e.g., one or more automatically-generated question recommendations that pertain to and/or are relevant to the second set of information) (e.g., in some embodiments, without displaying one or more automatically-generated question recommendations that are generated based on the first set of information) without displaying the first set of one or more automatically-generated question recommendations. Automatically generating question recommendations for a user allows a user to ask and/or submit these questions with fewer inputs. Furthermore, displaying different questions based on what information was being displayed to the user when the user invokes the agent also enhances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently.

In some embodiments, while displaying the one or more automatically-generated question recommendations (e.g., 618a, 618b, 618f, 618g, 630d, 630e, 618h, and/or 618i), the computer system detects, via the one or more input devices, a selection input (e.g., one or more user inputs) (e.g., one or more touch inputs, one or more gesture inputs, one or more hardware control inputs, and/or one or more spoken inputs) corresponding to selection of a first automatically-generated question recommendation of the one or more automatically-generated question recommendations (e.g., 628 and/or 636). In response to detecting the selection input corresponding to selection of the first automatically-generated question recommendation, the computer system displays, within the agent user interface (e.g., 616 and/or 622), a first response to the first automatically-generated question recommendation, wherein the first response is automatically generated (e.g., using an AI process or a generative AI process) (e.g., 630b-630c). In some embodiments, the first response is automatically generated by the computer system. In some embodiments, the first response is automatically generated by a remote computer system separate from the computer system. Automatically generating question recommendations for a user allows a user to ask and/or submit these questions with fewer inputs. Furthermore, doing so also enhances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently.

In some embodiments, while displaying the agent user interface (e.g., 616 and/or 622), the computer system detects, via the one or more input devices, a first query user input (e.g., 620b, 628, 636, 641, and/o 646) (e.g., one or more user inputs) (e.g., one or more touch inputs, one or more gesture inputs, one or more hardware control inputs, and/or one or more spoken inputs) corresponding to user entry of a first question (e.g., a user-typed first question, a spoken first question, and/or a selection of an automatically-generated question recommendation) that pertains to first set of information corresponding to the first type of health data. In response to detecting the first query user input, the computer system displays, within the agent user interface (e.g., 616 and/or 622), a response to the first question (e.g., 622b, 630b, 630c, 638b, 638f, and/or 638g), wherein: the response to the first question is automatically generated (e.g., using an AI process or a generative AI process); and the response includes first additional information pertaining to the first type of health data that is not displayed within the health summary user interface (e.g., 610). In some embodiments, responses generated by the agent include additional health information and/or health data that is not displayed within the health summary user interface. For example, in some embodiments, responses generated by the agent include automatically-generated visual content and/or generative visual content that are dynamically generated in response to the user query. Automatically generating responses to user queries enhances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently. Furthermore, providing the user with responses that include additional information that is not displayed in the health summary user interface allows the user to access this information without cluttering the limited display space in the health summary user interface.

In some embodiments, the first additional information includes one or more charts (e.g., 630c) (e.g., graphs) that includes biometric information collected from the user (e.g., by the computer system and/or by one or more sensors (e.g., that are part of the computer system or are separate from the computer system)) (e.g., blood pressure information, heart rate information, blood-oxygen level information, respiratory rate information, workout information, caloric intake information, exercise duration information, workout metric information, body temperature information, and/or sleep duration information). Automatically generating responses to user queries enhances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently. Furthermore, providing the user with responses that include additional information that is not displayed in the health summary user interface allows the user to access this information without cluttering the limited display space in the health summary user interface.

In some embodiments, the one or more charts (e.g., 630c) are dynamically generated in response to the first query user input (e.g., 628) (e.g., automatically generated (e.g., using an AI process or a generative AI process) in response to the first query user input). Automatically generating responses to user queries enhances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently. Furthermore, providing the user with responses that include additional information that is not displayed in the health summary user interface allows the user to access this information without cluttering the limited display space in the health summary user interface.

In some embodiments, the first additional information includes one or more workout recommendations (e.g., 638f and/or 638g), including a first workout recommendation corresponding to a first workout that, when selected, causes the computer system to initiate a process for starting a workout session corresponding to the first workout. In some embodiments, a workout recommendation corresponds to (e.g., represents) a workout (e.g., audio and/or video content that guides a user to perform a physical activity) (e.g., the first workout). In some embodiments, selecting a workout recommendation initiates a process for playback of a workout corresponding to the workout recommendation. In some embodiments, starting a workout session corresponding to the first workout comprises initiating recording and/or measuring of workout metrics corresponding to the workout session (e.g., workout duration, calories burned in the workout session, and/or average heart rate during the workout session). In some embodiments, while displaying the first workout recommendation within the agent user interface, the computer system detects one or more user inputs that include selection of the first workout recommendation; and in response to detecting the one or more user inputs that include selection of the first workout recommendation, the computer system initiates playback of the first workout (e.g., audio playback and/or video playback of the first workout) and/or starts a workout session corresponding to the first workout. In some embodiments, the one or more workout recommendations includes a second workout recommendation corresponding to a second workout different that, when selected, causes the computer system to initiate a process for starting a workout session corresponding to the second workout, wherein the second workout is different from the first workout. Automatically generating responses to user queries enhances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently. Furthermore, providing the user with responses that include additional information that is not displayed in the health summary user interface allows the user to access this information without cluttering the limited display space in the health summary user interface.

In some embodiments, the first additional information includes one or more selectable links (e.g., uniform resource links and/or directory links), including a first link that, when selected, causes the computer system to display a health user interface different from the health summary user interface (e.g., 610), wherein the health user interface includes biometric data collected from the user of the computer system (e.g., in some embodiments, chart 630c, when selected, causes computer system 600 to display a user interface that includes additional information about the user's historical daily steps data (e.g., historical daily steps data for the last year)) (e.g., collected by the computer system and/or by one or more sensors (e.g., that are part of the computer system or are separate from the computer system)) (e.g., blood pressure information, heart rate information, blood-oxygen level information, respiratory rate information, workout information, caloric intake information, exercise duration information, workout metric information, body temperature information, and/or sleep duration information). Providing the user with selectable links to access different health user interfaces allows the user to access these user interfaces with fewer inputs. Furthermore, doing so also enhances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently.

In some embodiments, displaying the agent user interface (e.g., 616 and/or 622) comprises displaying, concurrently with the one or more visual prompts (e.g., 618d and/or 622d) that prompt the user of the computer system to ask one or more questions, one or more previously-asked questions that have previously been entered by the user of the computer system (e.g., in some embodiments, in FIG. 6M, user interface 622 includes previous questions that have been entered by the user and/or previous responses generated by computer system 600 in response to those questions) (in some embodiments, user interface 622 is scrollable to view previous questions and/or responses). In some embodiments, displaying the agent user interface further comprises displaying, concurrently with the one or more visual prompts that prompts the user of the computer system to ask one or more questions and the one or more previously-asked questions, one or more responses to the one or more previously-asked questions, wherein the one or more responses were previously automatically generated (e.g., using an AI process or a generative AI process) in response to the one or more previously-asked questions. Displaying previously-asked questions within the agent user interface allows the user to access this information with fewer inputs. Furthermore, doing so also enhances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently.

In some embodiments, while displaying the agent user interface (e.g., 616 and/or 622), the computer system detects, via the one or more input devices, a first query input (e.g., one or more user inputs) (e.g., one or more touch inputs, one or more gesture inputs, one or more hardware control inputs, and/or one or more spoken inputs) that corresponds to user entry of a first respective question, wherein the first query input identifies a first image (e.g., a first picture and/or a first photograph) that is part of the first respective question (e.g., 652). In response to detecting the first query input, the computer system displays, within the agent user interface (e.g., 616 and/or 622), a first respective response that is responsive to the first respective question, wherein the first respective response is automatically generated (e.g., using an AI process or a generative AI process) in response to the first respective question (e.g., 638k, which is responsive to image 638j). In some embodiments, the first respective response is automatically generated by the computer system. In some embodiments, the first respective response is automatically generated by a remote computer system separate from the computer system. Automatically generating responses to user-entered questions enhances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently.

In some embodiments, the first image depicts a first set of food (e.g., 638j); and the first respective response (e.g., 638k) displays nutrition information (e.g., calories, amount of fat, amount of carbohydrates, amount of protein, amount of fiber, and/or amount of added sugar) pertaining to the first set of food, wherein first set of food is automatically identified (e.g., using an AI process or a generative AI process) based on the first image, and the nutrition information is automatically determined (e.g., using an AI process or a generative AI process) based on the first image. Automatically generating responses to user-entered questions enhances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently.

In some embodiments, in response to detecting the first query input (e.g., 638j and/or 652), the computer system (e.g., 600) logs (e.g., saving and/or storing) the nutrition information (e.g., to a nutrition log and/or a food intake log corresponding to the user of the computer system) (e.g., 638k). Automatically generating responses to user-entered questions enhances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently.

In some embodiments, the health summary user interface (e.g., 610) depicts one or more trends that are automatically identified (e.g., using an AI process or a generative AI process) based on biometric information collected from the user of the computer system (e.g., 612f, 612f-1, and/or 612f-2) (e.g., collected by the computer system and/or by one or more sensors (e.g., that are part of the computer system or are separate from the computer system)) (e.g., blood pressure information, heart rate information, blood-oxygen level information, respiratory rate information, workout information, caloric intake information, exercise duration information, workout metric information, body temperature information, and/or sleep duration information). In some embodiments, the one or more trends are automatically identified by the computer system. In some embodiments, the one or more trends are automatically identified by an externa computer system separate from the computer system. In some embodiments, the one or more trends are visually depicted as text identifying the one or more trends. In some embodiments, the one or more trends are visually depicted within a graph and/or a chart that depicts the one or more trends. Presenting different content within the health summary user interface and/or re-ordering information within the health summary user interface based on what information has been received since the last time the health summary user interface was displayed allows the user to access the most relevant information for the user with fewer and/or no additional user inputs. Furthermore, doing so also enhances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently.

In some embodiments, the determination that a first set of health information has been received for a user of the computer system (e.g., 600) since the health summary user interface (e.g., 610) was last displayed comprises a determination that a first set of laboratory test results (e.g., blood test results, urine analysis results, complete blood count, lipid panel, thyroid function test, diabetes test, antinuclear antibody test, c-reactive protein test, erythrocyte sedimentation rate test, genetic tests, blood glucose test, calcium blood test, blood urea nitrogen (BUN) test, creatine kinase test, CO2 blood test, serum potassium test, chloride blood test, globulin blood test, albumin blood test, alanine transaminase (ALT), alkaline phosphatase (ALP), bilirubin blood test, and/or aspartate transferase) has been received for the user of the computer system (e.g., the first set of laboratory results has been received from a laboratory testing service provider) since the health summary user interface was last displayed. In some embodiments, displaying the first set of information corresponding to the first type of health data based on the first set of health data comprises displaying, within the health summary user interface (e.g., 610), a representation of the first set of laboratory test results (e.g., 689a) (e.g., a representation of the first set of laboratory test results that indicates that the first set of laboratory test results has been received; a representation of the first set of laboratory test results that displays a subset of the first set of laboratory test results (e.g., a subset that is less than the full first set of laboratory test results); and/or a representation of the first set of laboratory test results that displays the first set of laboratory test results). Presenting different content within the health summary user interface and/or re-ordering information within the health summary user interface based on what information has been received since the last time the health summary user interface was displayed allows the user to access the most relevant information for the user with fewer and/or no additional user inputs. Furthermore, doing so also enhances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently.

In some embodiments, while displaying, via the one or more display generation components (e.g., 602) and within the first region (e.g., 611) of the health summary user interface (e.g., 610), the representation of the first set of laboratory test results (e.g., 689a), the computer system (e.g., 600) detects, via the one or more input devices, a selection input (e.g., 689b) (e.g., one or more touch inputs, one or more gesture inputs, one or more hardware control inputs, and/or one or more spoken inputs) corresponding to selection of the representation of the first set of laboratory test results. In response to detecting the selection input corresponding to selection of the representation of the first set of laboratory test results, the computer system displays, via the one or more display generation components (e.g., 602), a lab results user interface (e.g., 690) different from the health summary user interface (e.g., 610), wherein the lab results user interface displays additional information pertaining to the first set of laboratory test results that is not displayed in the representation of the first set of laboratory test results (e.g., 689a) and the health summary user interface (e.g., 610). In some embodiments, the lab results user interface displays one or more test results that are not displayed in the representation of the first set of laboratory test results and the health summary user interface. In some embodiments, the lab results user interface displays additional explanation about the first set of test results that is not displayed in the representation of the first set of laboratory test results and the health summary user interface. Allowing a user to select portions of the health summary user interface to access and/or display additional information allows the user to access this information without cluttering the health summary user interface with information that is not of interest to the user. Furthermore, doing so also enhances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently.

In some embodiments, the computer system (e.g., 600) detects, at a first time, via the one or more input devices, a first user request (e.g., 608) (e.g., one or more touch inputs, one or more gesture inputs, one or more hardware control inputs, and/or one or more spoken inputs) corresponding to a first request to display the health summary user interface (e.g., 610), wherein the first user request is received prior to receiving the first set of laboratory test results. In some embodiments, in response to detecting the first user request, the computer system displays, via the one or more display generation components, the health summary user interface (e.g., 610) with a third set of health information (e.g., 612a) within the first region (e.g., 611) of the health summary user interface (e.g., 610 in FIG. 6B and/or FIG. 6T), wherein the third set of health information is different from the representation of the first set of laboratory test results (e.g., 689a) (in some embodiments, the third set of health information does not pertain to and/or correspond to laboratory test results). In some embodiments, the computer system detects, at a second time subsequent to the first time, via the one or more input devices, a second user request (e.g., 608) (e.g., one or more touch inputs, one or more gesture inputs, one or more hardware control inputs, and/or one or more spoken inputs) corresponding to a second request to display the health summary user interface (e.g., 610), wherein the second user request is received subsequent to receiving the first set of laboratory test results. In some embodiments, in response to detecting the second user request, the computer system displays, via the one or more display generation components, the health summary user interface (e.g., 610) with the representation of the first set of laboratory test results (e.g., 689a) within the first region (e.g., 611) of the health summary user interface (e.g., 610 in FIG. 6FF). In some embodiments, in response to detecting the second user request, the computer system displays the health summary user interface with the third set of health information displayed within a second region of the health summary user interface different from the first region (e.g., in some embodiments, in FIG. 6FF, information 612a is displayed in a different portion of health summary user interface 610). In some embodiments, the first set of laboratory test results is displayed concurrently with the third set of health information. Presenting different content within the health summary user interface and/or re-ordering information within the health summary user interface based on what information has been received since the last time the health summary user interface was displayed allows the user to access the most relevant information for the user with fewer and/or no additional user inputs. Furthermore, doing so also enhances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently.

In some embodiments, the health summary user interface (e.g., 610) is generated by a first entity (e.g., a first corporate entity) (e.g., the health summary user interface is a product of the first entity and/or the health summary user interface is provided to the user of the computer system by a first entity); and the first set of laboratory results are generated by a second entity (e.g., a second corporate entity) different from the first entity (e.g., the first set of laboratory results are generated as a result of testing performed by the second entity), and are caused to be transmitted to the computer system (e.g., 600) by the second entity. In some embodiments, the health summary user interface is generated and/or displayed within a first product and/or a first application of the first entity, and the first set of laboratory test results are received from a third-party testing service (e.g., a second entity different from the first entity). Displaying lab results from a third party within the health summary user interface allows a user to access health information from a variety of sources within a single user interface, thereby reducing the number of inputs and applications required to access health information from multiple sources. Furthermore, doing so also enhances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently.

In some embodiments, in accordance with a determination that the user of the computer system (e.g., 600) has not completed medical laboratory testing for a threshold duration of time (e.g., the user has not completed medical laboratory testing and/or medical laboratory test results have not been received for the user of the computer system in the last 30 days, 60 days, 90 days, 120 days, or 365 days), the computer system displays, via the one or more display generation components (e.g., 602), a first recommendation (e.g., 654) for the user of the computer system to complete medical laboratory health testing. In some embodiments, in accordance with a determination that the user of the computer system has completed medical laboratory testing within the threshold duration of time, the computer system forgoes displaying the first recommendation. Automatically generating a reminder for the user of the computer system to complete medical laboratory testing provides the user with important information with fewer and/or no user inputs. Furthermore, doing so also enhances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently.

In some embodiments, the first recommendation (e.g., 654) is displayed within the health summary user interface (e.g., 610). Automatically generating a reminder for the user of the computer system to complete medical laboratory testing provides the user with important information with fewer and/or no user inputs. Furthermore, doing so also enhances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently.

In some embodiments, while displaying the first recommendation (e.g., 654), the computer system detects, via the one or more display generation components, a selection input (e.g., 655) (e.g., one or more touch inputs, one or more gesture inputs, one or more hardware control inputs, and/or one or more spoken inputs) corresponding to selection of the first recommendation. In some embodiments, in response to detecting the selection input, the computer system initiates a process for scheduling one or more medical laboratory tests (e.g., FIGS. 6T-6EE) (e.g., scheduling one or more medical laboratory tests with a third-party testing service). In some embodiments, the process for scheduling one or more medical laboratory tests includes selecting a testing location (e.g., FIG. 6W) (e.g., in some embodiments, the computer system displays a user interface (e.g., 664) with one or more medical laboratory testing location options for selection by the user). In some embodiments, the process for scheduling one or more medical laboratory tests includes selecting a testing date (e.g., FIG. 6Y) (e.g., in some embodiments, the computer system displays a user interface (e.g., 672) with one or more test date options (e.g., 674a) for selection by the user). In some embodiments, the process for scheduling one or more medical laboratory tests includes selecting a testing time (e.g., FIG. 6Y) (e.g., in some embodiments, the computer system displays a user interface (e.g., 672) with one or more test time options (e.g., 674b-1) for selection by the user). In some embodiments, the process for scheduling one or more medical laboratory tests includes selecting one or more medical laboratory tests to be performed (e.g., FIG. 6U) (e.g., in some embodiments, the computer system displays a user interface (e.g., 656) with one or more medical test options (e.g., 658c, 658d, 658e, and/or 658f) for selection by the user). Allowing the user to select the first reminder to initiate a process for scheduling one or more medical laboratory tests allows the user to schedule medical laboratory tests with fewer inputs. Furthermore, doing so also enhances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently.

Note that details of the processes described above with respect to method 700 (e.g., FIG. 7) are also applicable in an analogous manner to the methods described below. For example, method 900 optionally includes one or more of the characteristics of the various methods described above with reference to method 700. For example, in some embodiments, the first user interface recited in method 900 is accessible from the health summary user interface recited in method 700; and/or the same user health information is used to generate the health summary user interface recited in method 700 and the first notification and the first user interface recited in method 900. For brevity, these details are not repeated below.

FIGS. 8A-8H illustrate exemplary user interfaces for tracking and providing user health information, in accordance with some embodiments. The user interfaces in these figures are used to illustrate the processes described below, including the processes in FIG. 9.

FIG. 8A illustrates computer system 600, which is a smart phone with touch-sensitive display 602. At FIG. 8A, computer system 600 displays lock screen user interface 802. Lock screen user interface 802 includes complications 802a-802d, which correspond to and/or display information from different applications. For example, complication 802a displays data from a clock application and, in some embodiments, when selected, causes computer system 600 to open and/or display the clock application. Complication 802b displays data from a fitness application and, in some embodiments, when selected, causes computer system 600 to open and/or display the fitness application. Complication 802c corresponds to and/or displays data from a workout application and, in some embodiments, when selected, causes computer system 600 to open the workout application. Complication 802d displays data from a stopwatch application and, in some embodiments, when selected, causes computer system 600 to open and/or display the stopwatch application. Lock screen user interface 802 also includes flashlight option 802e which, when selected, causes computer system 600 to turn on or turn off a flashlight; and camera option 802f which, when selected, causes computer system 600 to display a camera user interface (e.g., camera user interface 650). Lock screen user interface 802 is displayed with indication 803, which indicates that computer system 600 has transitioned from a locked state to an unlocked state (e.g., based on biometric authentication or other authentication of a user of computer system 600).

At FIG. 8A, computer system 600 has received user health information indicating that the user of computer system 600 has three health-related activities scheduled for the day. In response to receiving this user health information, computer system 600 displays notification 804, which informs the user that they have three health-related activities scheduled for the day. At FIG. 8A, computer system 600 detects user input 806, which is a touch input (e.g., a tap input) corresponding to selection of notification 804.

At FIG. 8B, in response to detecting user input 806, computer system 600 displays digital assistant user interface 622, various embodiments of which were described above with reference to FIGS. 6A-6GG. At FIG. 8B, in response to user input 806, computer system 600 displays automatically generated responses 808a-808d within digital assistant user interface 622. Responses 808a-808d pertain to notification 804 and/or the health information that resulted in generation of notification 804, and displays additional information pertaining to notification 804 that was not displayed in notification 804. For example, responses 808b-808d specify and/or identify the three health-related activities that are scheduled for the user. Computer system 600 also displays response recommendations 810a-810b, which provide the user with one or more recommendations for responding to responses 808a-808d. For example, response recommendation 810a, when selected, causes computer system 600 to initiate a process for removing and/or canceling one or more of the three scheduled activities. Response recommendation 810b, when selected, causes computer system 600 to initiate a process for swapping one or more of the three scheduled activities with different activities. Option 810c, when selected, causes computer system 600 to generate and/or display additional response recommendations that pertain to notification 804. As discussed above with reference to FIGS. 6A-6GG, digital assistant user interface 622 also includes text field 822d and option 622e, which allow a user to manually enter a question for the digital assistant (e.g., via typing and/or spoken input).

FIG. 8C depicts a different example scenario in which computer system 600 has received user health information indicating that the user has just completed a run. For example, computer system 600 has received information from an external device (e.g., a smart watch) that indicates that the user has completed a run, and/or computer system 600 has received movement information and/or biometric information collected from the user that indicate that the user has completed a run. In response to receiving this user health information, computer system 600 generates and/or displays notification 812. At FIG. 8C, computer system 600 detects user input 814, which is a touch input (e.g., a tap input) corresponding to selection of notification 812.

At FIG. 8D, in response to detecting user input 814, computer system 600 displays digital assistant user interface 622, as well as responses 816a-816d, response recommendations 818a-818b, and option 818c. Responses 816a-816d pertain to notification 812 and/or the user health information that resulted in generation of notification 812, and display additional information that is not displayed in notification 812. For example, response 816c displays a map of the route the user traveled during their run, and response 816d provides average pace information for the user's run. In some embodiments, responses 816a-816d are automatically and/or dynamically generated in response to the user health information received by computer system 600 and/or in response to detecting user input 814. Response recommendation 818a, when selected, causes computer system 600 to display additional information about how much the user has improved in their runs. Response recommendation 818b, when selected, causes computer system 600 to display more information about how “average pace” is determined. Option 818c, when selected, causes computer system 600 to generate and/or display additional response recommendations that pertain to notification 812.

FIG. 8E depicts a different example scenario in which computer system 600 has received user health information indicating that the user has received three high heart rate alerts today (e.g., based on heart rate data collected using one or more sensors and/or one or more wearable devices (e.g., a smart watch and/or a heart rate sensor)). In response to receiving this user health information, computer system 600 generates and/or displays notification 820. At FIG. 8E, computer system 600 detects user input 822, which is a touch input (e.g., a tap input) corresponding to selection of notification 820.

At FIG. 8F, in response to detecting user input 822, computer system 600 displays digital assistant user interface 622, as well as responses 824a-824d, response recommendations 826a-826b, and option 826c. Responses 824a-824d pertain to notification 820 and/or the user health information that resulted in generation of notification 820, and display additional information that is not displayed in notification 820. For example, response 824b indicates that the user's resting heart rate was above 120 bpm; response 824c provides a chart of the user's heart rate measurements during the current day; and response 824d informs the user that repeated high heart rate alerts could be an indicator of an underlying issue. In some embodiments, responses 824a-824d are automatically and/or dynamically generated in response to the user health information received by computer system 600 and/or in response to detecting user input 822. Response recommendation 826a, when selected, causes computer system 600 to display additional information about how medications can affect heart rate. Response recommendation 826b, when selected, causes computer system 600 to display more information about additional symptoms the user should be aware of. Option 826c, when selected, causes computer system 600 to generate and/or display additional response recommendations that pertain to notification 820.

FIG. 8G depicts a different example scenario in which computer system 600 has received user health information indicating that the user has a scheduled meditation activity that is scheduled for the current time. In response to receiving this user health information, computer system 600 generates and/or displays notification 828a. Additionally, while the previous examples shown in FIGS. 8A-8F have included displaying notifications within lock screen user interface 802, it should be noted that in various embodiments, notifications can be displayed overlaid on different user interfaces. For example, on the right side of FIG. 8G, computer system 600 displays notification 828b overlaid on home screen user interface 606. At FIG. 8G, computer system 600 detects user input 830a, which is a touch input (e.g., a tap input) corresponding to selection of notification 828a and/or user input 830b, which is a touch input (e.g., a tap input) corresponding to selection of notification 828b.

At FIG. 8H, in response to detecting user input 830a and/or user input 830b, computer system 600 displays digital assistant user interface 622, as well as responses 832a-832b, response recommendations 834a-834b, and option 834c. Responses 832a-832b pertain to notification 828a and/or notification 828b and/or the user health information that resulted in generation of notification 828a and/or notification 828b, and display additional information that is not displayed in notification 828a and/or notification 828b. For example, response 832b, when selected, causes computer system 600 to initiate the 15-minute meditation that is scheduled for the current time (e.g., initiate playback of video and/or audio instructions for performing the 15-minute meditation activity). In some embodiments, responses 832a-832b are automatically and/or dynamically generated in response to the user health information received by computer system 600 and/or in response to detecting user input 830a and/or user input 830b. Response recommendation 834a, when selected, causes computer system 600 to set up a reminder to remind the user to perform the 15-minute meditation activity later (e.g., a reminder that will be displayed and/or output in one hour, or in two hours). Response recommendation 834b, when selected, causes computer system 600 to initiate a process for replacing the 15-minute meditation activity with a different activity (e.g., a different meditation activity and/or a different workout activity). Option 834c, when selected, causes computer system 600 to generate and/or display additional response recommendations that pertain to notification 828a and/or notification 828b.

FIG. 9 is a flow diagram illustrating a method for tracking and providing user health information using a computer system in accordance with some embodiments. Method 900 is performed at a computer system (e.g., 100, 300, 500, and/or 600) that is in communication with one or more display generation components (e.g., 602) (e.g., a display, a touch-sensitive display, and/or a display controller) and one or more input devices (e.g., 602) (e.g., a touch-sensitive surface, a touch-sensitive display, a button, a rotatable input mechanism, a depressible and rotatable input mechanism, a camera, an accelerometer, an inertial measurement unit (IMU), a heartrate sensor, a body temperature sensor, and/or a blood-oxygen level sensor). Some operations in method 900 are, optionally, combined, the orders of some operations are, optionally, changed, and some operations are, optionally, omitted.

As described below, method 900 provides an intuitive way for tracking and providing user health information. The method reduces the cognitive burden on a user for accessing health information, thereby creating a more efficient human-machine interface. For battery-operated computing devices, enabling a user to access health information faster and more efficiently conserves power and increases the time between battery charges.

The computer system (e.g., 600) receives (902) first user health information corresponding to a user of the computer system (e.g., in some embodiments, receiving first user health information comprises measuring user health information with one or more sensors) (e.g., biometric information; information pertaining to the physical health of the user; and/or information pertaining to physical metrics of the user). In response to receiving the first user health information (904), the computer system displays (906), via the one or more display generation components (e.g., 602), a first notification (e.g., 804, 812, 820, 828a, and/or 828b) pertaining to the first user health information (e.g., a pop-up notification, a banner notification, and/or a lock-screen notification) (e.g., in some embodiments, a first notification that includes and/or displays a first set of information that is determined, displayed, and/or included based on the first user health information). While displaying the first notification (908), the computer system detects (910), via the one or more input devices (e.g., 602), a first user input (e.g., 806, 814, 822, 830a, and/or 830b) corresponding to selection of the first notification (e.g., one or more inputs) (e.g., one or more touch inputs, one or more gesture inputs, one or more gaze inputs, one or more hardware control inputs, and/or one or more spoken inputs). In response to detecting the first user input (912), the computer system displays (914), via the one or more display generation components, a first user interface (e.g., 622), wherein the first user interface includes: additional user health information (e.g., 808b, 808c, 808d, 816c, 816d, 824b, 824c, 824d, and/or 832b) pertaining to the user of the computer system that was not displayed in the first notification (e.g., additional user health information that pertains to the first user health information, that is determined based on the first user health information, and/or that is displayed based on that first user health information); and a first prompt (e.g., 622d) prompting the user of the computer system to ask a question. Automatically generating a health notification for a user allows the user to view this information with fewer and/or no user inputs. Displaying the notification also provides the user with improved visual feedback about a state of the computer system (e.g., that the computer system has received the first user health information). Additionally, allowing the user to select the notification to display a user interface that presents the user with more information about the notification and prompts the user to ask a question allows the user to access this information while saving display space when the notification is displayed. Furthermore, doing so also enhances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently.

In some embodiments, displaying the additional user health information (e.g., 808b, 808c, 808d, 816c, 816d, 824b, 824c, 824d, and/or 832b) pertaining to the user of the computer system that was not displayed in the first notification (e.g., 804, 812, 820, 828a, and/or 828b) comprises: in accordance with a determination that the first notification includes a first type of health information (e.g., a first category of health information and/or a first set of health information), displaying, within the first user interface (e.g., 622), first additional user health information that pertains to the user of the computer system and pertains to the first type of health information and was not displayed in the first notification (e.g., 808b-808d in FIG. 8B); and in accordance with a determination that the first notification includes a second type of health information (e.g., a second category of health information and/or a second set of health information) different from the first type of health information (and, optionally, without including the first type of health information) displaying, within the first user interface, second additional user health information that pertains to the user of the computer system and pertains to the second type of health information and was not displayed in the first notification and is different from the first additional user health information (e.g., 816c-816d in FIG. 8D) (and, optionally, without displaying the first additional user health information). In some embodiments, different types of notifications, when selected, cause the computer system to display the first user interface with different content (e.g., content that is determined and/or selected based on the content of the selected notification). Automatically generating a health notification for a user allows the user to view this information with fewer and/or no user inputs. Displaying the notification also provides the user with improved visual feedback about a state of the computer system (e.g., that the computer system has received the first user health information). Additionally, allowing the user to select the notification to display a user interface that presents the user with more information about the notification and prompts the user to ask a question allows the user to access this information while saving display space when the notification is displayed. Furthermore, doing so also enhances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently.

In some embodiments, displaying the first user interface (e.g., 622) further includes displaying, concurrently with the additional user health information (e.g., 808b, 808c, 808d, 816c, 816d, 824b, 824c, 824d, and/or 832b) and the first prompt (e.g., 622d), one or more automatically-generated (e.g., using an AI process or a generative AI process) question recommendations (e.g., 810a, 810b, 818a, 818b, 826a, 826b, 834a, and/or 834b) (e.g., one or more automatically-generated question recommendations that are selectable by the user) that pertain to the additional user health information (e.g., in some embodiments, that are generated based on the additional user health information (e.g., that are automatically generated (e.g., using an AI process or a generative AI process) to pertain to and/or be relevant to the additional user health information)). Automatically generating question recommendations for a user allows a user to ask and/or submit these questions with fewer inputs. Furthermore, doing so also enhances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently.

In some embodiments, the first user health information pertains to one or more activities that have been automatically scheduled for the user of the computer system (e.g., FIG. 8B) (e.g., one or more workouts and/or meditations that have been automatically scheduled for the user of the computer system (e.g., in some embodiments, a workout and/or meditation is selected for the user from a set of workouts and/or meditations based on user-specified guidelines but without user selection of a specific workout and/or meditation)); the first notification (e.g., 804) is generated in response to receiving the first user health information that pertains to the one or more activities that have been automatically scheduled for the user of the computer system; and the first notification (e.g., 804) pertains to the one or more activities that have been automatically scheduled for the user of the computer system (e.g., the first notification notifies the user of the one or more activities that have been automatically scheduled for the user of the computer system). Automatically generating a health notification for a user allows the user to view this information with fewer and/or no user inputs. Displaying the notification also provides the user with improved visual feedback about a state of the computer system (e.g., that the computer system has received the first user health information). Additionally, allowing the user to select the notification to display a user interface that presents the user with more information about the notification and prompts the user to ask a question allows the user to access this information while saving display space when the notification is displayed. Furthermore, doing so also enhances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently.

In some embodiments, the first user interface (e.g., 622) includes additional information about the one or more activities (e.g., 808b-808d) that have been automatically scheduled for the user of the computer system that was not displayed in the first notification (e.g., 804). Allowing the user to select the notification to display a user interface that presents the user with more information about the notification and prompts the user to ask a question allows the user to access this information while saving display space when the notification is displayed. Furthermore, doing so also enhances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently.

In some embodiments, while displaying the first user interface (e.g., 622), the computer system detects, via the one or more input devices, one or more user inputs (e.g., one or more touch inputs, one or more gesture inputs, one or more gaze inputs, one or more hardware control inputs, and/or one or more spoken inputs) corresponding to a user request to modify a first activity of the one or more activities that have been automatically scheduled for the user of the computer system (e.g., user input selecting option 810b and/or user input into text field 622d, via keyboard 644, and/or spoken input requesting to switch out one or more of the scheduled activities). In response to detecting the one or more user inputs corresponding to the user request to modify the first activity, the computer system swaps the first activity with a second activity different from the first activity (e.g., a second workout and/or a second meditation different from the first activity (e.g., a first workout and/or a first meditation)). Allowing the user to request modification of a scheduled activity within the first user interface allows the user to perform these operations with fewer inputs. Furthermore, doing so also enhances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently.

In some embodiments, while displaying the first user interface (e.g., 622), the computer system detects, via the one or more input devices, one or more user inputs (e.g., one or more touch inputs, one or more gesture inputs, one or more gaze inputs, one or more hardware control inputs, and/or one or more spoken inputs) corresponding to a user request to modify a first respective activity of the one or more activities that have been automatically scheduled for the user of the computer system (e.g., user input selecting option 810b and/or user input into text field 622d, via keyboard 644, and/or spoken input requesting to switch out one or more of the scheduled activities). In response to detecting the one or more user inputs corresponding to the user request to modify the first respective activity, the computer system displays, within the first user interface, a first follow-up question pertaining to the user request to modify the first respective activity (e.g., an automatically generated follow-up question (e.g., automatically generated using an AI process and/or a generative AI process)) (e.g., similar to follow-up question 638c that asks the user to clarify how long they would like their workout to be in FIG. 6M, in some embodiments, in response to user input selecting option 810b in FIG. 8B, computer system 600 displays a follow-up question asking which activity the user would like to replace and/or asking the user what type of activity they would like to swap in). In some embodiments, subsequent to displaying the first follow-up question, the computer system detects one or more user inputs responsive to the first follow-up question; and in response to detecting the one or more user inputs responsive to the first follow-up question, the computer system swaps the first respective activity with a second respective activity different from the first respective activity (e.g., based on the one or more user inputs responsive to the first follow-up question). Allowing the user to request modification of a scheduled activity within the first user interface allows the user to perform these operations with fewer inputs. Furthermore, doing so also enhances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently.

In some embodiments, displaying the additional user health information pertaining to the user of the computer system that was not displayed in the first notification comprises displaying a start activity option (e.g., 832b) that, when selected, causes the computer system to initiate a first activity of the one or more activities that have been automatically scheduled for the user of the computer system (e.g., initiate a workout and/or a meditation; and/or initiate playback of a workout and/or a meditation). In some embodiments, while displaying the first user interface, including the start activity option, the computer system detects a selection input corresponding to selection of the start activity option; and in response to detecting the selection input, the computer system initiates the first activity. Providing the user with an option to start a scheduled activity from within the first user interface allows the user to perform these operations with fewer user inputs. Furthermore, doing so also enhances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently.

In some embodiments, the first user health information pertains to one or more workouts that have been completed by the user of the computer system (e.g., FIG. 8C); the first notification (e.g., 812) is generated in response to receiving the first user health information that pertains to the one or more workouts that have been completed by the user of the computer system; and the first notification (e.g., 812) pertains to the one or more workouts that have been completed by the user of the computer system (e.g., the first notification indication, congratulates, and/or recognizes the one or more workouts that have been completed by the user of the computer system). Automatically generating a health notification for a user allows the user to view this information with fewer and/or no user inputs. Displaying the notification also provides the user with improved visual feedback about a state of the computer system (e.g., that the computer system has received the first user health information). Additionally, allowing the user to select the notification to display a user interface that presents the user with more information about the notification and prompts the user to ask a question allows the user to access this information while saving display space when the notification is displayed. Furthermore, doing so also enhances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently.

In some embodiments, displaying the additional user health information pertaining to the user of the computer system that was not displayed in the first notification comprises displaying additional workout information pertaining to the one or more workouts that have been completed by the user of the computer system that was not displayed in the first notification (e.g., 816c and/or 816d) (e.g., workout metrics, average pace, route map, comparison of workout to previous workouts, calories burned, and/or workout duration). Allowing the user to select the notification to display a user interface that presents the user with more information about the notification and prompts the user to ask a question allows the user to access this information while saving display space when the notification is displayed. Furthermore, doing so also enhances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently.

In some embodiments, displaying the first user interface (e.g., 622) further includes displaying, concurrently with the additional user health information (e.g., 816c and/or 816d) and the first prompt (e.g., 622d), one or more automatically-generated (e.g., using an AI process or a generative AI process) question recommendations (e.g., 818a and/or 818b) (e.g., one or more automatically-generated question recommendations that are selectable by the user) that pertain to the one or more workouts that have been completed by the user of the computer system (e.g., in some embodiments, that are generated based on the additional user health information (e.g., that are automatically generated (e.g., using an AI process or a generative AI process) to pertain to and/or be relevant to the additional user health information)). Automatically generating question recommendations for a user allows a user to ask and/or submit these questions with fewer inputs. Furthermore, doing so also enhances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently.

In some embodiments, the first user health information pertains to a fitness goal (e.g., in some embodiments, in FIGS. 8C-8D, the user has a fitness goal for achieving a target run time and/or a target run pace; and/or in some embodiments, in FIGS. 8G-8H, the user has a fitness goal for completing a target number of meditations in a week) corresponding to the user of the computer system (e.g., a user-defined fitness goal and/or an automatically-assigned fitness goal) (e.g., a workout streak (e.g., consecutive days and/or weeks completing a workout); a daily calories burned goal; a weekly calories burned goal; a daily workout goal; a weekly workout goal; and/or a monthly workout goal); the first notification (e.g., 812, 828a, and/or 828b) is generated in response to receiving the first user health information that pertains to the fitness goal corresponding to the user of the computer system; and the first notification pertains to the fitness goal corresponding to the user of the computer system (e.g., the first notification identifies the fitness goal and/or encourages the user to work on the fitness goal; the first notification identifies one or more activities that can be completed by the user to progress towards the fitness goal; and/or the first notification congratulates and/or recognizes completion of the fitness goal). Automatically generating a health notification for a user allows the user to view this information with fewer and/or no user inputs. Displaying the notification also provides the user with improved visual feedback about a state of the computer system (e.g., that the computer system has received the first user health information). Additionally, allowing the user to select the notification to display a user interface that presents the user with more information about the notification and prompts the user to ask a question allows the user to access this information while saving display space when the notification is displayed. Furthermore, doing so also enhances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently.

In some embodiments, displaying the first user interface (e.g., 622) further includes displaying, concurrently with the additional user health information (e.g., 816c, 816d, and/or 832b) and the first prompt (e.g., 622d), one or more automatically-generated (e.g., using an AI process or a generative AI process) question recommendations (e.g., 818a, 818b, 834a, and/or 834b) (e.g., one or more automatically-generated question recommendations that are selectable by the user) that pertain to the fitness goal corresponding to the user of the computer system (e.g., in some embodiments, that are generated based on the additional user health information (e.g., that are automatically generated (e.g., using an AI process or a generative AI process) to pertain to and/or be relevant to the additional user health information)). Automatically generating question recommendations for a user allows a user to ask and/or submit these questions with fewer inputs. Furthermore, doing so also enhances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently.

In some embodiments, the first user health information pertains to a health-based alert (e.g., warning and/or potential health risk) (e.g., high heart rate, low blood oxygen, high blood pressure, and/or low blood pressure) for the user of the computer system (e.g., FIGS. 8E-8F), wherein the health-based alert is determined based on biometric information collected from the user of the computer system (e.g., by the computer system (e.g., using one or more sensors of the computer system) and/or by one or more sensors separate from the computer system); the first notification (e.g., 820) is generated in response to receiving the first user health information that pertains to the health alert for the user of the computer system; and the first notification (e.g., 820) pertains to the health alert for the user of the computer system (e.g., the first notification identifies the health alert and/or is indicative of the health alert). Automatically generating a health notification for a user allows the user to view this information with fewer and/or no user inputs. Displaying the notification also provides the user with improved visual feedback about a state of the computer system (e.g., that the computer system has received the first user health information). Additionally, allowing the user to select the notification to display a user interface that presents the user with more information about the notification and prompts the user to ask a question allows the user to access this information while saving display space when the notification is displayed. Furthermore, doing so also enhances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently.

In some embodiments, displaying the additional user health information pertaining to the user of the computer system that was not displayed in the first notification (e.g., 820) comprises displaying additional information pertaining to the health-based alert (e.g., 824b, 824c, and/or 824d) for the user of the computer system that was not displayed in the first notification (e.g., 820). Allowing the user to select the notification to display a user interface that presents the user with more information about the notification and prompts the user to ask a question allows the user to access this information while saving display space when the notification is displayed. Furthermore, doing so also enhances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently.

In some embodiments, the additional information pertaining to the health-based alert includes one or more biometric measurements (e.g., 824c) pertaining to the health-based alert (e.g., heart rate measurements, blood pressure measurements, and/or blood oxygen level measurements) (e.g., measured by one or more sensors of the computer system and/or one or more sensors separate from the computer system). Allowing the user to select the notification to display a user interface that presents the user with more information about the notification and prompts the user to ask a question allows the user to access this information while saving display space when the notification is displayed. Furthermore, doing so also enhances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently.

In some embodiments, displaying the first user interface (e.g., 622) further includes displaying, concurrently with the additional user health information (e.g., 824b, 824c, and/or 824d) and the first prompt (e.g., 622d), one or more automatically-generated (e.g., using an AI process or a generative AI process) question recommendations (e.g., 826a and/or 826b) (e.g., one or more automatically-generated question recommendations that are selectable by the user) that pertain to the health-based alert for the user of the computer system (e.g., in some embodiments, that are generated based on the additional user health information (e.g., that are automatically generated (e.g., using an AI process or a generative AI process) to pertain to and/or be relevant to the additional user health information)). Automatically generating question recommendations for a user allows a user to ask and/or submit these questions with fewer inputs. Furthermore, doing so also enhances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently.

Note that details of the processes described above with respect to method 900 (e.g., FIG. 9) are also applicable in an analogous manner to the methods described above. For example, method 700 optionally includes one or more of the characteristics of the various methods described above with reference to method 900. For example, in some embodiments, the first user interface recited in method 900 is accessible from the health summary user interface recited in method 700; and/or the same user health information is used to generate the health summary user interface recited in method 700 and the first notification and the first user interface recited in method 900. For brevity, these details are not repeated below.

The foregoing description, for purpose of explanation, has been described with reference to specific embodiments. However, the illustrative discussions above are not intended to be exhaustive or to limit the invention to the precise forms disclosed. Many modifications and variations are possible in view of the above teachings. The embodiments were chosen and described in order to best explain the principles of the techniques and their practical applications. Others skilled in the art are thereby enabled to best utilize the techniques and various embodiments with various modifications as are suited to the particular use contemplated.

Although the disclosure and examples have been fully described with reference to the accompanying drawings, it is to be noted that various changes and modifications will become apparent to those skilled in the art. Such changes and modifications are to be understood as being included within the scope of the disclosure and examples as defined by the claims.

Some embodiments described herein can include use of artificial intelligence and/or machine learning systems (sometimes referred to herein as the AI/ML systems). The use can include collecting, processing, labeling, organizing, analyzing, recommending and/or generating data. Entities that collect, share, and/or otherwise utilize user data should provide transparency and/or obtain user consent when collecting such data. The present disclosure recognizes that the use of the data in the AI/ML systems can be used to benefit users. For example, the data can be used to train models that can be deployed to improve performance, accuracy, and/or functionality of applications and/or services. Accordingly, the use of the data enables the AI/ML systems to adapt and/or optimize operations to provide more personalized, efficient, and/or enhanced user experiences. Such adaptation and/or optimization can include tailoring content, recommendations, and/or interactions to individual users, as well as streamlining processes, and/or enabling more intuitive interfaces. Further beneficial uses of the data in the AI/ML systems are also contemplated by the present disclosure.

The present disclosure contemplates that, in some embodiments, data used by AI/ML systems includes publicly available data. To protect user privacy, data may be anonymized, aggregated, and/or otherwise processed to remove or to the degree possible limit any individual identification. As discussed herein, entities that collect, share, and/or otherwise utilize such data should obtain user consent prior to and/or provide transparency when collecting such data. Furthermore, the present disclosure contemplates that the entities responsible for the use of data, including, but not limited to data used in association with AI/ML systems, should attempt to comply with well-established privacy policies and/or privacy practices.

For example, such entities may implement and consistently follow policies and practices recognized as meeting or exceeding industry standards and regulatory requirements for developing and/or training AI/ML systems. In doing so, attempts should be made to ensure all intellectual property rights and privacy considerations are maintained. Training should include practices safeguarding training data, such as personal information, through sufficient protections against misuse or exploitation. Such policies and practices should cover all stages of the AI/ML systems development, training, and use, including data collection, data preparation, model training, model evaluation, model deployment, and ongoing monitoring and maintenance. Transparency and accountability should be maintained throughout. Such policies should be easily accessible by users and should be updated as the collection and/or use of data changes. User data should be collected for legitimate and reasonable uses of the entity and not shared or sold outside of those legitimate uses. Further, such collection and sharing should occur through transparency with users and/or after receiving the informed consent of the users. Additionally, such entities should consider taking any needed steps for safeguarding and securing access to such data and ensuring that others with access to the data adhere to their privacy policies and procedures. Further, such entities should subject themselves to evaluation by third parties to certify, as appropriate for transparency purposes, their adherence to widely accepted privacy policies and practices. In addition, policies and/or practices should be adapted to the particular type of data being collected and/or accessed and tailored to a specific use case and applicable laws and standards, including jurisdiction-specific considerations.

In some embodiments, AI/ML systems may utilize models that may be trained (e.g., supervised learning or unsupervised learning) using various training data, including data collected using a user device. Such use of user-collected data may be limited to operations on the user device. For example, the training of the model can be done locally on the user device so no part of the data is sent to another device. In other implementations, the training of the model can be performed using one or more other devices (e.g., server(s)) in addition to the user device but done in a privacy preserving manner, e.g., via multi-party computation as may be done cryptographically by secret sharing data or other means so that the user data is not leaked to the other devices.

In some embodiments, the trained model can be centrally stored on the user device or stored on multiple devices, e.g., as in federated learning. Such decentralized storage can similarly be done in a privacy preserving manner, e.g., via cryptographic operations where each piece of data is broken into shards such that no device alone (i.e., only collectively with another device(s)) or only the user device can reassemble or use the data. In this manner, a pattern of behavior of the user or the device may not be leaked, while taking advantage of increased computational resources of the other devices to train and execute the ML model. Accordingly, user-collected data can be protected. In some implementations, data from multiple devices can be combined in a privacy-preserving manner to train an ML model.

In some embodiments, the present disclosure contemplates that data used for AI/ML systems may be kept strictly separated from platforms where the AI/ML systems are deployed and/or used to interact with users and/or process data. In such embodiments, data used for offline training of the AI/ML systems may be maintained in secured datastores with restricted access and/or not be retained beyond the duration necessary for training purposes. In some embodiments, the AI/ML systems may utilize a local memory cache to store data temporarily during a user session. The local memory cache may be used to improve performance of the AI/ML systems. However, to protect user privacy, data stored in the local memory cache may be erased after the user session is completed. Any temporary caches of data used for online learning or inference may be promptly erased after processing. All data collection, transfer, and/or storage should use industry-standard encryption and/or secure communication.

In some embodiments, as noted above, techniques such as federated learning, differential privacy, secure hardware components, homomorphic encryption, and/or multi-party computation among other techniques may be utilized to further protect personal information data during training and/or use of the AI/ML systems. The AI/ML systems should be monitored for changes in underlying data distribution such as concept drift or data skew that can degrade performance of the AI/ML systems over time.

In some embodiments, the AI/ML systems are trained using a combination of offline and online training. Offline training can use curated datasets to establish baseline model performance, while online training can allow the AI/ML systems to continually adapt and/or improve. The present disclosure recognizes the importance of maintaining strict data governance practices throughout this process to ensure user privacy is protected.

In some embodiments, the AI/ML systems may be designed with safeguards to maintain adherence to originally intended purposes, even as the AI/ML systems adapt based on new data. Any significant changes in data collection and/or applications of an AI/ML system use may (and in some cases should) be transparently communicated to affected stakeholders and/or include obtaining user consent with respect to changes in how user data is collected and/or utilized.

Despite the foregoing, the present disclosure also contemplates embodiments in which users selectively restrict and/or block the use of and/or access to data. That is, the present disclosure contemplates that hardware and/or software elements can be provided to prevent or block access to data. For example, in the case of some services, the present technology should be configured to allow users to select to “opt in” or “opt out” of participation in the collection of data during registration for services or anytime thereafter. In another example, the present technology should be configured to allow users to select not to provide certain data for training the AI/ML systems and/or for use as input during the inference stage of such systems. In yet another example, the present technology should be configured to allow users to be able to select to limit the length of time data is maintained or entirely prohibit the use of their data for use by the AI/ML systems. In addition to providing “opt in” and “opt out” options, the present disclosure contemplates providing notifications relating to the access or use of personal information. For instance, a user can be notified when their data is being input into the AI/ML systems for training or inference purposes, and/or reminded when the AI/ML systems generate outputs or make decisions based on their data.

The present disclosure recognizes AI/ML systems should incorporate explicit restrictions and/or oversight to mitigate against risks that may be present even when such systems having been designed, developed, and/or operated according to industry best practices and standards. For example, outputs may be produced that could be considered erroneous, harmful, offensive, and/or biased; such outputs may not necessarily reflect the opinions or positions of the entities developing or deploying these systems. Furthermore, in some cases, references to third-party products and/or services in the outputs should not be construed as endorsements or affiliations by the entities providing the AI/ML systems. Generated content can be filtered for potentially inappropriate or dangerous material prior to being presented to users, while human oversight and/or ability to override or correct erroneous or undesirable outputs can be maintained as a failsafe.

The present disclosure further contemplates that users of the AI/ML systems should refrain from using the services in any manner that infringes upon, misappropriates, or violates the rights of any party. Furthermore, the AI/ML systems should not be used for any unlawful or illegal activity, nor to develop any application or use case that would commit or facilitate the commission of a crime, or other tortious, unlawful, or illegal act. The AI/ML systems should not violate, misappropriate, or infringe any copyrights, trademarks, rights of privacy and publicity, trade secrets, patents, or other proprietary or legal rights of any party, and appropriately attribute content as required. Further, the AI/ML systems should not interfere with any security, digital signing, digital rights management, content protection, verification, or authentication mechanisms. The AI/ML systems should not misrepresent machine-generated outputs as being human-generated.

As described above, one aspect of the present technology is the gathering and use of data available from various sources to improve the delivery to users of health information or any other content that may be of interest to them. The present disclosure contemplates that in some instances, this gathered data may include personal information data that uniquely identifies or can be used to contact or locate a specific person. Such personal information data can include demographic data, location-based data, telephone numbers, email addresses, social network IDs, home addresses, data or records relating to a user's health or level of fitness (e.g., vital signs measurements, medication information, exercise information), date of birth, or any other identifying or personal information.

The present disclosure recognizes that the use of such personal information data, in the present technology, can be used to the benefit of users. For example, the personal information data can be used to deliver targeted content that is of greater interest to the user.

Accordingly, use of such personal information data enables users to have calculated control of the delivered content. Further, other uses for personal information data that benefit the user are also contemplated by the present disclosure. For instance, health and fitness data may be used to provide insights into a user's general wellness, or may be used as positive feedback to individuals using technology to pursue wellness goals.

The present disclosure contemplates that the entities responsible for the collection, analysis, disclosure, transfer, storage, or other use of such personal information data will comply with well-established privacy policies and/or privacy practices. In particular, such entities should implement and consistently use privacy policies and practices that are generally recognized as meeting or exceeding industry or governmental requirements for maintaining personal information data private and secure. Such policies should be easily accessible by users, and should be updated as the collection and/or use of data changes. Personal information from users should be collected for legitimate and reasonable uses of the entity and not shared or sold outside of those legitimate uses. Further, such collection/sharing should occur after receiving the informed consent of the users. Additionally, such entities should consider taking any needed steps for safeguarding and securing access to such personal information data and ensuring that others with access to the personal information data adhere to their privacy policies and procedures. Further, such entities can subject themselves to evaluation by third parties to certify their adherence to widely accepted privacy policies and practices. In addition, policies and practices should be adapted for the particular types of personal information data being collected and/or accessed and adapted to applicable laws and standards, including jurisdiction-specific considerations. For instance, in the US, collection of or access to certain health data may be governed by federal and/or state laws, such as the Health Insurance Portability and Accountability Act (HIPAA); whereas health data in other countries may be subject to other regulations and policies and should be handled accordingly. Hence different privacy practices should be maintained for different personal data types in each country.

Despite the foregoing, the present disclosure also contemplates embodiments in which users selectively block the use of, or access to, personal information data. That is, the present disclosure contemplates that hardware and/or software elements can be provided to prevent or block access to such personal information data. For example, in the case of health information delivery services, the present technology can be configured to allow users to select to “opt in” or “opt out” of participation in the collection of personal information data during registration for services or anytime thereafter. In another example, users can select not to provide health information or other personal information. In yet another example, users can select to limit the length of time user health information is maintained or entirely prohibit the collection of user health information. In addition to providing “opt in” and “opt out” options, the present disclosure contemplates providing notifications relating to the access or use of personal information. For instance, a user may be notified upon downloading an app that their personal information data will be accessed and then reminded again just before personal information data is accessed by the app.

Moreover, it is the intent of the present disclosure that personal information data should be managed and handled in a way to minimize risks of unintentional or unauthorized access or use. Risk can be minimized by limiting the collection of data and deleting data once it is no longer needed. In addition, and when applicable, including in certain health related applications, data de-identification can be used to protect a user's privacy. De-identification may be facilitated, when appropriate, by removing specific identifiers (e.g., date of birth, etc.), controlling the amount or specificity of data stored (e.g., collecting location data a city level rather than at an address level), controlling how data is stored (e.g., aggregating data across users), and/or other methods.

Therefore, although the present disclosure broadly covers use of personal information data to implement one or more various disclosed embodiments, the present disclosure also contemplates that the various embodiments can also be implemented without the need for accessing such personal information data. That is, the various embodiments of the present technology are not rendered inoperable due to the lack of all or a portion of such personal information data. For example, content can be selected and delivered to users by inferring preferences based on non-personal information data or a bare minimum amount of personal information, such as the content being requested by the device associated with a user, other non-personal information available to the content delivery services, or publicly available information.

1-56. (canceled)

57. A computer system configured to communicate with one or more display generation components and one or more input devices, comprising:
one or more processors; and
memory storing one or more programs configured to be executed by the one or more processors, the one or more programs including instructions for:
receiving first user health information corresponding to a user of the computer system;
in response to receiving the first user health information, displaying, via the one or more display generation components, a first notification pertaining to the first user health information;
while displaying the first notification, detecting, via the one or more input devices, a first user input corresponding to selection of the first notification; and
in response to detecting the first user input, displaying, via the one or more display generation components, a first user interface, wherein the first user interface includes:
additional user health information pertaining to the user of the computer system that was not displayed in the first notification; and
a first prompt prompting the user of the computer system to ask a question.

58. The computer system of claim 57, wherein displaying the additional user health information pertaining to the user of the computer system that was not displayed in the first notification comprises:
in accordance with a determination that the first notification includes a first type of health information, displaying, within the first user interface, first additional user health information that pertains to the user of the computer system and pertains to the first type of health information and was not displayed in the first notification; and
in accordance with a determination that the first notification includes a second type of health information different from the first type of health information displaying, within the first user interface, second additional user health information that pertains to the user of the computer system and pertains to the second type of health information and was not displayed in the first notification and is different from the first additional user health information.

59. The computer system of claim 57, wherein displaying the first user interface further includes displaying, concurrently with the additional user health information and the first prompt, one or more automatically-generated question recommendations that pertain to the additional user health information.

60. The computer system of claim 57, wherein:
the first user health information pertains to one or more activities that have been automatically scheduled for the user of the computer system;
the first notification is generated in response to receiving the first user health information that pertains to the one or more activities that have been automatically scheduled for the user of the computer system; and
the first notification pertains to the one or more activities that have been automatically scheduled for the user of the computer system.

61. The computer system of claim 60, wherein the first user interface includes additional information about the one or more activities that have been automatically scheduled for the user of the computer system that was not displayed in the first notification.

62. The computer system of claim 60, the one or more programs further including instructions for:
while displaying the first user interface, detecting, via the one or more input devices, one or more user inputs corresponding to a user request to modify a first activity of the one or more activities that have been automatically scheduled for the user of the computer system; and
in response to detecting the one or more user inputs corresponding to the user request to modify the first activity, swapping the first activity with a second activity different from the first activity.

63. The computer system of claim 60, the one or more programs further including instructions for:
while displaying the first user interface, detecting, via the one or more input devices, one or more user inputs corresponding to a user request to modify a first respective activity of the one or more activities that have been automatically scheduled for the user of the computer system; and
in response to detecting the one or more user inputs corresponding to the user request to modify the first respective activity, displaying, within the first user interface, a first follow-up question pertaining to the user request to modify the first respective activity.

64. The computer system of claim 60, wherein displaying the additional user health information pertaining to the user of the computer system that was not displayed in the first notification comprises displaying a start activity option that, when selected, causes the computer system to initiate a first activity of the one or more activities that have been automatically scheduled for the user of the computer system.

65. The computer system of claim 57, wherein:
the first user health information pertains to one or more workouts that have been completed by the user of the computer system;
the first notification is generated in response to receiving the first user health information that pertains to the one or more workouts that have been completed by the user of the computer system; and
the first notification pertains to the one or more workouts that have been completed by the user of the computer system.

66. The computer system of claim 65, wherein displaying the additional user health information pertaining to the user of the computer system that was not displayed in the first notification comprises displaying additional workout information pertaining to the one or more workouts that have been completed by the user of the computer system that was not displayed in the first notification.

67. The computer system of claim 65, wherein displaying the first user interface further includes displaying, concurrently with the additional user health information and the first prompt, one or more automatically-generated question recommendations that pertain to the one or more workouts that have been completed by the user of the computer system.

68. The computer system of claim 57, wherein:
the first user health information pertains to a fitness goal corresponding to the user of the computer system;
the first notification is generated in response to receiving the first user health information that pertains to the fitness goal corresponding to the user of the computer system; and
the first notification pertains to the fitness goal corresponding to the user of the computer system.

69. The computer system of claim 68, wherein displaying the first user interface further includes displaying, concurrently with the additional user health information and the first prompt, one or more automatically-generated question recommendations that pertain to the fitness goal corresponding to the user of the computer system.

70. The computer system of claim 57, wherein:
the first user health information pertains to a health-based alert for the user of the computer system, wherein the health-based alert is determined based on biometric information collected from the user of the computer system;
the first notification is generated in response to receiving the first user health information that pertains to the health alert for the user of the computer system; and
the first notification pertains to the health alert for the user of the computer system.

71. The computer system of claim 70, wherein displaying the additional user health information pertaining to the user of the computer system that was not displayed in the first notification comprises displaying additional information pertaining to the health-based alert for the user of the computer system that was not displayed in the first notification.

72. The computer system of claim 71, wherein the additional information pertaining to the health-based alert includes one or more biometric measurements pertaining to the health-based alert.

73. The computer system of claim 71, wherein displaying the first user interface further includes displaying, concurrently with the additional user health information and the first prompt, one or more automatically-generated question recommendations that pertain to the health-based alert for the user of the computer system.

74. A non-transitory computer-readable storage medium storing one or more programs configured to be executed by one or more processors of a computer system that is in communication with one or more display generation components and one or more input devices, the one or more programs including instructions for:
receiving first user health information corresponding to a user of the computer system;
in response to receiving the first user health information, displaying, via the one or more display generation components, a first notification pertaining to the first user health information;
while displaying the first notification, detecting, via the one or more input devices, a first user input corresponding to selection of the first notification; and
in response to detecting the first user input, displaying, via the one or more display generation components, a first user interface, wherein the first user interface includes:
additional user health information pertaining to the user of the computer system that was not displayed in the first notification; and
a first prompt prompting the user of the computer system to ask a question.

75. A method, comprising:
at a computer system that is in communication with one or more display generation components and one or more input devices:
receiving first user health information corresponding to a user of the computer system;
in response to receiving the first user health information, displaying, via the one or more display generation components, a first notification pertaining to the first user health information;
while displaying the first notification, detecting, via the one or more input devices, a first user input corresponding to selection of the first notification; and
in response to detecting the first user input, displaying, via the one or more display generation components, a first user interface, wherein the first user interface includes:
additional user health information pertaining to the user of the computer system that was not displayed in the first notification; and
a first prompt prompting the user of the computer system to ask a question.
